(19) United States
(12) Patent Application Publication
(10) Pub. No.: US 2024/0216502 A1
(43) Pub. Date: Jul. 4, 2024

(54) UNIVERSAL VACCINE PLATFORM

(71) Applicants: Hugh MASON, Phoenix, AZ (US); Andrew DIAMOS, Tempe, AZ (US); Mary PARDHE, Phoenix, AZ (US); Brandon FAVRE, Los Gatos, CA (US)

(72) Inventors: Hugh MASON, Phoenix, AZ (US); Andrew DIAMOS, Tempe, AZ (US); Mary PARDHE, Phoenix, AZ (US); Brandon FAVRE, Los Gatos, CA (US)

(73) Assignee: ARIZONA BOARD OF REGENTS ON BEHALF OF ARIZONA STATE UNIVERSITY, Scottsdale, AZ (US)

(21) Appl. No.: 18/504,962

(22) Filed: Nov. 8, 2023

Related U.S. Application Data
(63) Continuation of application No. 17/367,296, filed on Jul. 2, 2021, now Pat. No. 11,865,174.
Application No. 17/367,296 is a continuation of application No. 16/404,698, filed on May 6, 2019, now Pat. No. 11,058,766.
(60) Provisional application No. 62/667,414, filed on May 4, 2018.
(60) Provisional application No. 62/821,599, filed on Mar. 21, 2019.

(51) Int. Cl. A61K 39/29; A61K 39/12; A61K 39/145; A61P 31/20; C12N 7/00; A61K 39/00

(52) U.S. Cl. CPC A61K 39/292; A61K 39/12; A61K 39/145; A61P 31/20; C12N 7/00; A61K 2039/505; A61K 2039/5258; A61K 2039/575; A61K 2039/6056; A61K 2039/70; C12N 2710/20023; C12N 2710/20034; C12N 2710/20071; C12N 2730/10123; C12N 2730/10134; C12N 2730/10171; C12N 2760/16123; C12N 2760/16134; C12N 2760/16171; C12N 2770/24134; C12N 2770/24171

(57) ABSTRACT

The disclosure relates to vaccination compositions, for example, against human papillomavirus, Zika virus, and flu virus. The disclosure also relates to vectors for producing the virus-like particles and immune complex platforms of the vaccination compositions.

### RELATED APPLICATIONS

This application is a continuation of U.S. patent application Ser. No. 17/367,296, filed Jul. 2, 2021, which is a continuation of U.S. patent application Ser. No. 16/404,698, filed May 6, 2019, which claims the benefit of and priority to U.S. provisional patent application 62/667,414, filed May 4, 2018, and U.S. provisional patent application 62/821,599, filed Mar. 21, 2019, the contents of each of which are incorporated herein by reference in their entireties.

### STATEMENT REGARDING FEDERALLY SPONSORED RESEARCH OR DEVELOPMENT

This invention was made with government support under R33 AI101329 and U19 AI062150 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 217,522-byte XML file named “Seq_List.xml” created on Mar. 21, 2024.

### TECHNICAL FIELD

The disclosure relates to vaccine platform comprising a virus-like particle (VLP) formed from hepatitis B core antigens and/or a recombinant immune complex (RIC).

### BACKGROUND

Documentation on using inoculation as a strategy to provide protection against smallpox dates as early as hundreds of years before common era. These early reports of inoculation involved exposure to tissue diseased with smallpox (powered smallpox scabs or fluid from smallpox pustule). After Edward Jenner's report that inoculation with pus from a cowpox sore became widely accepted in the late 18^{th} century, vaccine researchers turn to inoculants with reduced virulence. With improved understanding of the genetic makeup of pathogens and advancements in bioengineering, vaccination strategies moved away from attenuated vaccination or inactivated vaccination where an actual pathogen was used. Instead, recombinant proteins that induce an antigenic response against a pathogen without the risk of an infection developing have become focus of vaccination strategies. Some of the efforts in recombinant vaccine development have focused on the design of and evaluating the effectiveness of recombinant immune complexes (RICs) and virus-like particles (VLPs) against pathogens that currently lack an effective or efficient vaccine.

Recombinant immune complexes (RICs), fundamentally, are composed of immunoglobulin molecules specific for a desired antigen that are fused to the same antigen that the antibody is specific for (Chargelegue et al., 2005). Specifically, the parts of an RIC are an antibody, linked via its C-terminus, to an antigen that is followed by an epitope tag for the antibody. This allows for the binding region of one antibody to bind to the antigen recombinantly fused to another antibody, resulting in the formation of large, highly immunogenic antibody-antigen complexes (Chargelegue et al., 2005). RICs can be engineered into ‘universal vaccine platforms’ through the use of antibodies specific for an epitope tag, which allows for the same antibody to be used regardless of the antigen so long as the antibody's corresponding epitope tag is expressed on the antigen (Mason et al., 2016) (FIG. 1). Thus, RIC can potentiate the immunogenicity of a given antigen. However, the requirement that the antigen needs to be fused to the C-terminus of the antibody of the RIC prevents antigens with inaccessible N-termini to easily be used in a RIC without disrupting native antigenic conformation.

RICs take advantage of existing immunological mechanisms by utilizing antibodies' natural interaction with Fcγ receptors (Fridman, 1991; Van den Hoecke et al., 2017), which results in the phagocytosis and processing of the RICs and the target antigens contained within. Additionally, the increased concentration of antigens within the RIC can allow for increased B cell-receptor cross-linking, increasing B cell stimulation and activation (Avalos & Ploegh, 2014). Thus, RICs can mimic a natural immune complex and effect activation of the immune system. RICs have been evaluated as treatment platforms for diseases like Ebola fever (Phoolcharoen et al., 2011) and tuberculosis (Pepponi et al., 2014) as well as vaccine platforms for HIV (Hioe et al., 2009) and Dengue fever (Kim et al., 2015).

Virus-like particles (VLPs) are non-infectious, protein-based nanoparticles derived from virus capsids that self-assemble into virus-like structures and can be modified to recombinantly express vaccine antigens, making them an adaptable vaccine platform for combatting a myriad of diseases (Rohovie et al., 2017). One of the VLP vaccines against human papillomavirus have been commercially available in the United States since the 2006 approval of Merck & Co.'s Gardasil, and more recently, potent, VLP-based vaccines have been shown significant efficacy against diseases like Zika virus (Yang et al., 2017), norovirus (Diamos & Mason, 2018), and even influenza (Pushko et al., 2017; Ramirez et al., 2018), each of which make use of the hepatitis B core antigen (HBcAg) as the platform for their VLPs.

HBcAg offers several advantages as a vaccine platform: it is both a T-cell dependent and T-cell independent antigen (Milich et al., 1986), it preferentially evokes a ThI response instead of a Th2 response like other hepatitis B antigens (Milich et al., 1995), is a potent activator of macrophages (Cooper et al., 2005), and can be used effectively to present heterologous antigens without compromising the assembly or integrity of the HBc VLP (Schodel et al., 1992). Delivering HBcAg VLPs with non-HBc antigens at the c/el loop of HBc a-helical spike, a region also known as the ‘major insertion region’ (MIR), has been shown to evoke immune responses to both the HBcAg and the inserted antigen (Whitacre et al., 2009). To form VLPs, HBcAg monomers first assemble into dimers, which in turn form full VLPs when expressed in eukaryotes (Pumpens et al., 2001; Mechtcheriakova et al., 2006). This technique has been refined to allow for larger proteins to be expressed through the use of the ‘tandem core’ approach, which involves fusing two HBcAg reading frames together, which enables the expression of foreign antigens on both, neither, or only one of the MIRs of the HBc tandem core (Peyret et al., 2015) (FIG. 2). Opting to express foreign antigens on only one of the two spike regions of the HBc dimer reduces the steric hindrance between foreign antigens, which consequently increases the maximum size of the potential foreign antigens that can be included in the VLP (Peyret et al., 2015).

VLPs offer several advantages over more traditional vaccination approaches. To start, they can self-assemble to resemble the structure of their native virus, providing the immune system with a more authentic target and consequently improving VLPs' immunogenicity (Chackerian, 2014). Further, because they lack genomic information, they are unable to replicate, improving the safety of any VLP delivered as a vaccine. Additionally, owing to their fundamental nature of being solely a recombinant protein, they are able to be produced at much faster rates than live-attenuated and inactivated viruses, as there is no need to use production systems, like eggs, that would support virus replication. This simultaneously lowers the cost and opens the doors to a wide variety of different production methods that can be chosen based on the needs for glycosylation, folding, speed, etc. desired for any given VLP.

One of hindrances of recombinant vaccines include difficulties in economically producing sufficient amounts of these recombinant proteins. One approach to answering this need is the use of plants as a production vector for recombinant vaccines (Favre, 2018). The production of valuable and viable biopharmaceuticals and vaccine antigens in plants is well-documented as being a cost-effective alternative to other means of biopharmaceutical production (Streatfield et al., 2001; Fischer & Emans, 2000; Tiwari et al., 2009; Rybicki, 2010). Plants can be grown abundantly and cheaply, providing a large source of inexpensive biomass without the need for costly bioreactors used by traditional fermentation-based systems (Chen and Davis 2016). Recent economic analyses have found substantial cost reductions for biological products made in plant-based systems compared to traditional systems (Tuse et al. 2014; Nandi et al. 2016). Furthermore, unlike mammalian systems, plants do not harbor animal pathogens, and have limited potential for contamination with bacterial endotoxins.

The use of geminiviral vectors has been demonstrated to significantly increase the yield of proteins expressed in plants systems. Geminiviral vectors allow for the insertion of desired genes into a self-replicating plant virus vector (Davies & Stanley, 1989; Stanley, 1993), which facilitates the production of vaccine antigens in plants (Chen et al., 2011). Geminiviral replication proteins amplify gene expression through the use of cellular DNA replication machinery in the nucleus, where the DNA uses soluble histones to form a ‘viral minichromosome’ (separate of the host genome) (Hefferon, 2014; Paprotka et al., 2015). This amplification of genes of interest is achieved through the inclusion of geminiviral replicon elements in the expression cassette. Specifically, the inclusion of the genes Rep and Rep A, as well as geminiviral short and long intergenic regions, in cis allows for the genes of interest to be amplified once delivered into the plant (Lazarowitz & Shepherd, 2008; Hefferon, 2014). Delivery of the expression cassette containing both the genes of interest and geminiviral replicon elements is enhanced through the use of the hypervirulent EHA105 strain of Agrobacterium tumefaciens, which can be used to transfer an expression cassette flanked by the left and right border sequences of the A. tumefaciens Ti plasmid into plants.

Plants are prime candidates for producing recombinant vaccines, as their glycosylation patterns can be modified to improve vaccine efficacy. For instance, some biopharmaceutical production methods inadvertently fucosylate their products, which can be counterintuitive as fucose inhibits binding of various targets by Fc gamma RIII receptors, which decreases the efficacy of antibody-based therapeutics (Shields et al., 2002). However, engineering plants to feature knocked out fucosylation pathways, as well as upregulated GnGn glycosylation (which increases binding to Fcγ RIIIA receptors (Maverakis et al., 2015), can increase the efficacy of plant-expressed biopharmaceuticals. Specifically, GnGn N. benthamiana plants have been engineered to produce human N-glycosylation by downregulating the endogenous β1,2-xylosylation (XylT) and α1,3-fucosyltransferase (FucT) genes (Strasser et al., 2008). This is key, as fucosylation inhibits FcγR recognition which reduces the efficacy of immunoglobulin-based treatments (Niwa et al., 2005), and β1,2-xylosylation and core α1,3-fucose are absent from humans entirely, which could provoke unwanted immune responses against non-GnGn plant-produced therapeutics. The lack of α1,6-fucose, which is normally present in humans but not in plants, may actually be beneficial, as the lack of fucose improves antibody-dependent cellular cytotoxicity (Shields et al., 2002), making GnGn plants the optimal production system for plant-produced biopharmaceuticals and a viable production vector for a universal influenza A vaccine.

### SUMMARY

The disclosure relates to compositions and methods for vaccination against viruses. The vaccination compositions include a virus-like particle (VLP) and a recombinant immune complex (RIC). The methods relate to increasing the immunogenicity of virus antigens, and methods of generating an immune response in a mammalian subject, e.g., an immune response against human papillomavirus, influenza virus, and zika virus. The methods of increasing the immunogenicity of virus antigens comprise presenting virus antigens to an immune system on a VLP or a RIC. Accordingly, in some aspects, the disclosure relates to the VLPs and RICs described herein. The methods of generating an immune response in a mammalian subject against human papillomavirus, influenza virus, and zika virus comprise administering to a subject a VLP presenting an antigen from these viruses and/or at least one RIC comprising an antigen from these viruses. In some aspects, the methods of generating an immune response in a mammalian subject against human papillomavirus (HPV), influenza virus, and zika virus comprises administering to the mammalian subject a composition comprising the VLP and the RIC.

In certain embodiments, the VLPs described herein comprise a first hepatitis B virus core antigen (HBcAg) monomer, a second HBcAg monomer, and a first fragment of a virus protein, wherein the first fragment of a virus protein is linked to the major insertion region of the second HBcAg monomer. The first HBcAg monomer and the second HBcAg monomer forms a HBcAg dimer, which forms the VLP core. In some aspects, the first fragment of the virus protein is linked between amino acid residue 77 and amino acid residue 78 of the second HBcAg monomer.

In certain embodiments, the RIC comprises an immunoglobulin heavy chain, an epitope tag that can bind to the immunoglobulin heavy chain, and a second fragment of a virus protein. In some aspects, the second fragment of the virus protein is linked to the C-terminus of the immunoglobulin heavy chain, which forms a C-RIC. Thus, in some embodiments, the epitope tag is linked to the C-terminus of the second fragment of the virus protein. While in other aspects, the second fragment of the virus protein is linked to the N-terminus of the immunoglobulin heavy chain, which forms a N-RIC. In such embodiments, the epitope tag is linked to the C-terminus of the immunoglobulin heavy chain. In some embodiments, the RIC further comprises an immunoglobulin light chain.

In certain embodiments of the RIC, the epitope tag is an ebola antigen. In such embodiments, the epitope tag is preferably the 6D8 epitope tag and the immunoglobulin heavy chain of the RIC is preferably the immunoglobulin heavy chain of humanized 6D8 monoclonal antibody.

Certain vaccination compositions are configured for vaccination against multiple subtypes of HPV, and they comprise the virus protein HPV minor capsid protein L2 (GenBank Accession No. AGH32604.1). In such compositions, the first fragment of the virus protein comprises at least 100 continuous amino acid residues from the first 200 amino acid residues from the N-terminus of HPV minor capsid protein L2. In certain embodiments, the first fragment of the virus protein comprises residues 14-122 of HPV16 minor capsid protein L2. In some aspects, the second fragment of the virus protein comprises at least 8 continuous amino acids from the first 200 amino acid residues from the N-terminus of HPV minor capsid protein L2. In certain embodiments, the second fragment of the virus protein comprises at least one peptide sequence from the HPV minor capsid protein L2 selected from the group consisting of: amino acid residues 17-36, amino acid residues 56-75, amino acid residues 65-85, and amino acid residues 96-115.

In certain vaccination compositions configured for vaccination against influenza virus, the virus protein is the ectodomain of influenza matrix protein 2 and the first fragment of the virus protein and the second fragment of the virus protein comprises the amino acid sequence SLLTEVETPIRNEWGCRCNDSSD (SEQ ID NO. 9). In certain embodiments, first fragment of the virus protein and the second fragment of the virus protein comprises the amino acid sequence set forth in SEQ ID NO. 10.

In particular vaccination compositions configured for vaccination against zika virus, the virus protein is selected from the group consisting of: zika virus E protein domain 3 protein (ZE3), zika virus fusion loop antigen (ZE), and zika virus soluble envelope protein (Zse). In some aspects, the first fragment of the virus protein and the second fragment of the virus protein comprise amino acid residues 591-696 of GenBank Accession No. AMC13911.1. In other aspects, the first fragment of the virus protein and the second fragment of the virus protein comprise amino acid residues 352-412 of GenBank Accession No. AMC13911.1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an exemplary diagram of universal recombinant immune complex (RIC) targeting an antigen. In this example, the antigen is linked to the C-terminus of the immunoglobulin heavy chain. The epitope tag of one immunoglobulin is bound to the binding site of other immunoglobulins, forming complexes that contain the target antigen.

FIG. 2 is an exemplary diagram showing the assembly of virus-like particles (VLPs) with a core of hepatitis B core antigen (HBcAg) using the tandem core approach.

FIG. 3A-3D depict, in accordance with certain embodiments, schematic representation of exemplary BeYDV plant expression vectors used to express human papillomavirus (HPV) VLPs and RICs. For FIGS. 3A and 3C, the region between the two LIRs is circularized and amplified to high copy number in the plant nucleus, serving as transcription templates for the VLP and RIC, respectively. Abbreviations: RB, the agrobacterium right T-DNA border region; PinII3′, the terminator from the potato proteinase inhibitor II gene; P19, the RNA silencing suppressor from tomato bushy stunt virus; P35S, the 35S promoter from cauliflower mosaic virus; LIR, the long intergenic region from BeYDV; NbPsaK 5′, the truncated 5′ UTR from NbPsaK; VLP, either L2-fused or unfused hepatitis B core antigen; Heavy-L2, the mAb 6D8 gamma (heavy) chain with C-terminal L2 fusion; Light, the 6D8 kappa (light) chain; IEU, the tobacco extensin terminator with intron; MAR, the rb7 matrix attachment region; SIR, the short intergenic region from BeYDV; Rep/RepA, the replication proteins from BeYDV; LB, the agrobacterium T-DNA left border region. FIG. 3B depicts a vector map for a plant expression plasmid encoding a HPV VLP presenting residues 14-122 of HPV minor capsid protein L2. The nucleic acid sequence of pBYR2eK2M-HBcheL2ic is set forth in SEQ ID NO. 43. FIG. 3D depicts a vector map for a plant expression plasmid encoding a HPV RIC comprising residues 14-122 of HPV minor capsid protein L2. The nucleic acid sequence of pBYR11eMa-h6D8-L2 is set forth in SEQ ID NO. 44.

FIG. 4A-4C depict, in accordance with certain embodiments, the expression of VLP and RIC that display L2 antigen. FIG. 4a shows leaves of Nicotinana benthamiana were agroinfiltrated with VLP vectors and imaged under visible light at 4-5 DPI to monitor tissue cell death. FIG. 4B depicts the SDS-PAGE and western blot of homogenized and clarified agroinfiltrated leaf tissue. Arrows indicate VLP bands. FIG. 4C depicts western blot analysis of soluble and insoluble samples from RIC agroinfiltrated leaves.

FIGS. 5A-5E depict, in accordance with certain embodiments, the purification of fully assembled HPV L2 VLP and RIC. FIG. 5A depicts the sucrose gradient sedimentation profile of crude extracts of leaf tissue agroinfiltrated with either L2-fused or unfused HBche. Following sedimentation through 10%-20%-30%-40%-50% sucrose layers, fractions were analyzed by SDS-PAGE, using ImageJ software to quantify the band intensity. FIG. 5B depicts SDS-PAGE of pooled material from either sucrose gradient purified VLP or protein G affinity purified RIC. Far right lane shows western blot analysis of purified RIC with anti-L2 as probe. FIGS. 5C and 5D depict electron microscopy of dialyzed peak sucrose fractions after negative staining with 0.5% uranyl acetate, bar=20 nm. FIG. 5E shows C1q binding of HBche-L2 RIC. Purified human IgG (left) or purified HBche-L2 RIC (right) were used at 0.5 mg/ml (1:1) and 3-fold serial dilutions.

FIG. 6 depicts, in accordance with certain embodiments, L2-specific IgG titers in mice receiving VLP or RIC. Balb/c mice were immunized subcutaneously, using alum as adjuvant, with three doses of either PBS, L2, VLP, RIC, or VLP/RIC mixed 1:1. Except for the PBS control group, each dose delivered 5 μg total L2 and was administered in three-week intervals. Blood samples were collected before each dose and analyzed for L2-specific antibodies by endpoint titer ELISA. The y-axis shows the geometric mean titers (GMT) and error bars show 95% confidence intervals. Two stars (**) indicates p values <0.05 and three stars (***) indicates p values <0.001.

FIG. 7, in accordance with certain embodiments, compares IgG1 and IgG2a production of mice receiving VLP or RIC in accordance with certain embodiments. Serum samples after the final dose were diluted as indicated and analyzed by ELISA specific for IgG1 or IgG2a antibodies. Columns represent means±the standard error from 8 serum samples. The inset table shows the relative ratio of IgG2a to IgG1 for each group as compared to L2 alone.

FIG. 8, in accordance with certain embodiments, compares in vitro neutralization of HPV pseudovirons with L2, VLP, RIC, or VLP/RIC. Sera of mice immunized with L2, VLP, RIC, or VLP/RIC were diluted and used to neutralize HPV16 pseudovirons before infection of 293FT cells. Infectivity is shown as relative luminesce; diminished luminescence is evidence of impaired infection of 293FT cells. Horizontal lines indicate the group mean. (**) indicates p value <0.005; (***) indicates p value <0.001.

FIGS. 9A-9B depict, in accordance with certain embodiments, the vector encoding the M2e-RIC, pBYR11eMa-h6D8M2e. FIG. 9A shows a simplified schematic of the vector, while FIG. 9B shows expression vector map. Pin2 3′ is the 3′ end of the Pin2 gene's promoter. The p19 gene encodes the p19 protein of the tomato bushy stunt virus, a suppressor of post-translational gene silencing (Chen et al., 2011). The TMV 5′ UTR is a viral translational enhancer that is spurred on by the binding of HSP101, which recruits the translational initiation factors elF4G and elF3 (Gaille 2002, Diamos et al., 2016). P35s is a viral promoter sequence. NbPsaK2T (Nb=N. benthamiana, PsaK=photosystem I reaction center subunit, T=truncated) 1-63 5′ UTR is used as a leader sequence and is directly upstream of the initiation codon; previous work in this laboratory found that this was the optimal 5′ UTR for expressing vaccine antigens in plants in a comparison of 23 5′ UTRs (Diamos et al., 2016). The human IgG 6D8 Heavy Chain gene (shown as Human IgG 6D8 Heavy Chain H2IS in FIG. 9B) encodes the heavy chain of the humanized anti-ebola antibody specific for ebola glycoprotein epitope 6D8, while the 6D8-K3 gene encodes the antibody's light chain. This is linked by a glycine-serine linker to dimeric 2×M2e, with each copy of M2e being linked to the other by a glycine-serine linker. 6D8 (gp 357-371) is the ebola glycoprotein epitope 6D8, which serves as the epitope binding tag for this RIC. Ext-3′ FL is the extensin gene's 3′ flanking region, which was used as a terminator and was previously found through research in this laboratory to enhance protein production relative to other more common terminators (Diamos et al., 2016). The Rb7 MAR (matrix attachment region) increases the likelihood and magnitude of transgene expression in plants (Halweg et al., 2005). C1/C2 encodes the geminiviral bean yellow dwarf virus (BeYDV) Rep/RepA proteins, which require the BeYDV LIR and SIR to function properly, as the LIR contains a bi-directional promoter and stem-loop structure essential for the initiation of rolling-circle replication and the SIR is the origin of C-strand synthesis and supplies termination and polyadenylation signals (Chen et al., 2011). The Rep/RepA proteins facilitate the formation of a transgene-containing ‘viral minichromosome’ within the plant cells' nuclei and subsequently enhances protein expression (Chen et al., 2011). Finally, the T-DNA left and right borders are the borders of the expression cassette that is transferred to the cells of the plant by Agrobacterium tumefaciens.

FIGS. 10A-10B depict, in accordance with certain embodiments, the vector encoding the M2e-VLP, pBYR2eK2M-HBcheM2e. FIG. 10A shows a simplified schematic of the vector, while FIG. 10B shows expression vector map. The vector uses many of the same elements as the M2e-RIC vector, pBYR11eMa-h6D8M2e (Figs. (A-9B), but features HBc1-149, HBc1-77, and HBc78-176 genes encoding the heptatis B core antigen assembled in a ‘tandem core’ fashion, in the place of the human IgG heavy and light chain sequences and ACT3a 3′ as a stabilizing agent of mRNAs that increases the lifespan of the transcript (Diamos & Mason, 2018).

FIGS. 11A-11B depict, in accordance with certain embodiments, Coomassie staining of RIC plant extract showing successful expression of M2e-RIC. The approximate size of the M2e RIC is 164 kDa, with the heavy chain-2×M2e-h6D8 fusion being roughly 57 kDa in size and the anti-6D8 light chain being roughly 25 kDa in size. In FIG. 11A, samples were run under non-reducing conditions, with the bands of the size of the RIC being clearly present in the elutions from the protein G resin and the removal of other contaminants post-acid precipitation and post-chromatography being evident. Bands of a larger size than the expected 164 kDa could indicate immune complex formation. For FIG. 111B, all samples were run under reducing conditions (indicated by a ‘-R’, with the exception of an IgG standard, which was run under both reducing and non-reducing conditions. The heavy chain of the RIC, when reduced, has a greater size than that of the IgG standard's, which is due to the heavy chain of the RIC being fused to the M2e dimer and 6D8 epitope tag, which adds an additional 7 kDa to its size. Additionally, the light chains of the IgG standard and the RIC groups are of the same size, indicating that the 2×M2e and epitope tag were not expressed on the light chain of the RIC.

FIGS. 12A-12B depict, in accordance with certain embodiments, western blotting of RIC and VLP samples. For FIG. 12A, the gel was run concurrently with gel in FIG. 11A and samples were run under non-reducing conditions. The samples were probed with anti-h6D8 antibody, and a clear signal is seen in both the crude and the elution fractions, demonstrating the presence of the epitope tag. Bands are present at the expected 164 kDa range, and larger bands may suggest immune complex formation. For FIG. 12B, samples of the M2e-RIC, M2e-VLP, and an anti-M2e antibody, MAb65, were run under both reducing and non-reducing conditions. The samples were probed with MAb65 to confirm the presence of M2e, and the detection antibody was anti-human light chain. A single band is present in the M2e-RIC reducing conditions group that is around the expected size of the M2e-RIC's heavy chain-2×M2e-epitope tag fusion. Because the samples were run under reducing conditions, no band of the heavy chain's size would have appeared if it did not contain M2e, as the anti-human light chain probe would not have bound to the heavy chain on its own. The probe did bind to the light chains of the M2e RIC and MAb65 further down the gel (not pictured). Additionally, bands are present in both samples of the VLP, confirming the presence of M2e in the VLP.

FIG. 13, shows, in accordance with certain embodiments, an exemplary Transmission Electron Microscopy image of M2e-VLP. Of the formation of VLPs indicated by this image, the particle in the upper-right quartile shows the clearest indication of proper virus-like particle formation.

FIGS. 14A-14C depict, in accordance with certain embodiments, mouse antibody titers post-vaccination. Total anti-M2e antibody titers were higher, on average, in mice that received both the M2e-RIC and the M2e VLP vaccines than in mice that received only the M2e-VLP vaccine (Dose 1: 1.17*10⁴ vs 6.25*10³; Dose 2: 1*10⁶ vs 3.8*10⁵; Dose 3: 4.43*10⁶ vs 2.25*10⁶, respectively. IgG2a to IgG1 ratios, however, were higher in the group receiving only the M2e-VLP vaccine compared to the group receiving both vaccines. Although dilutions were different for IgG1 and IgG2a titer ELISAs, preventing direct comparison, the VLP/RIC group demonstrated IgG2a:IgG1 ratios that were 64% lower relative to those of the VLP-alone group when the ratio of IgG2a to IgG1 is arbitrarily set at 1.0.

FIGS. 15A-15B show, in accordance with certain embodiments, an analysis of cytokine production in mouse splenocytes. Splenocyte analysis revealed that the combination VLP/RIC vaccine induced lower levels of both IL-4 and IFN-7 when compared to both unvaccinated and the VLP alone group.

FIG. 16 depicts, in accordance with certain embodiments, a vector map for a plant expression plasmid encoding a zika virus VLP presenting domain III of the zika virus E protein (K591-T696 of Accession No. AMC13911) (ZE3 VLP). The nucleic acid sequence of pBYe3R2K2Mc-BAHBcheZE3 is set forth in SEQ ID NO. 34.

FIG. 17 depicts, in accordance with certain embodiments, a vector map for a plant expression plasmid encoding a zika virus VLP presenting the zika virus fusion loop antigen (E352-S412 of Accession No. AMC13911). The nucleic acid sequence of pBYe3R2K2Mc-BAHBcheZE62-122 is set forth in SEQ ID NO. 35.

FIG. 18 depicts, in accordance with certain embodiments, a vector map for a plant expression plasmid encoding a zika virus RIC where the zika virus fusion loop antigen (E352-S412 of Accession No. AMC13911) is linked to the h6D8 antibody at the N-terminus of its heavy chain. The nucleic acid sequence of pBYRIleM-h6D8-ZEFL62 is set forth in SEQ ID NO. 36.

FIG. 19 depicts, in accordance with certain embodiments, a vector map for a plant expression plasmid encoding a zika virus RIC where the domain III of the zika virus E protein (K591-T696 of Accession No. AMC13911) is linked to the h6D8 antibody at the N-terminus of its heavy chain. The nucleic acid sequence of pBYRIleM-h6D8ZE3 is set forth in SEQ ID NO. 37.

FIG. 20 depicts, in accordance with certain embodiments, a vector map for a plant expression plasmid encoding a zika virus RIC where the C-terminus of domain III of the zika virus E protein (K591-T696 of Accession No. AMC13911) is linked to the N-terminus of the heavy chain of h6D8 antibody. The C-terminus of the heavy chain of the antibody is linked to the 6D8 epitope tag. The nucleic acid sequence of pBYRIleMa-BAZE3-Hgp371 is set forth in SEQ ID NO. 38.

FIGS. 21A-21D depict, in accordance with certain embodiments, purification of ZE3 C-RIC and N-RIC. Following protein G column chromatography of the ZE3 C-RIC and N-RIC, samples of the C-RIC elutions were analyzed by an SDS-PAGE gel stained with Coomassie (FIG. 21A) and by a western blot probed anti-human IgG+HRP (FIG. 21B). Samples of the N-RIC were also analyzed by an SDS-PAGE gel stained with Coomassie (FIG. 21C) and a western blot probed anti-human IgG (Fc only)+HRP (FIG. 21D). Both reducing and non-reducing conditions were tested. Abbreviations: R, reducing conditions, and NR, non-reducing conditions

FIGS. 22A-22B depict, in accordance with certain embodiments, partial purification of the ZE3 VLP. After sucrose gradient sedimentation, a fraction was analyzed on an SDS-PAGE gel stained with Coomassie (FIG. 22A) and a western blot probed with a polyclonal rabbit anti-Zika envelope antibody and detected with a goat anti-rabbit+HRP antibody (FIG. 22B).

FIGS. 23A-23B depict, in accordance with certain embodiments, purification of a recombinant immune complex where the domain III of the zika virus E protein (K591-T696 of Accession No. AMC13911, labeled ZE3 in the figure) or the zika soluble ectodomain E protein (amino acids 291-693, labeled ZsE in the figure) is linked to the antibody at the N-terminus of its heavy chain. The Western blot results showed appropriate assembly of both the ZsE N-RIC and ZDIII N-RIC (FIG. 23A). FIG. 23B depicts Coomassie-stained gel with purified recombinant immune complex having the domain III of the zika virus E protein (K591-T696 of Accession No. AMC13911, labeled ZE3 in the figure) linked to the antibody at the N-terminus of its heavy chain (ZE3 N-RIC) under reducing (R) and non-reducing (NR) conditions.

FIG. 24 depicts, in accordance with certain embodiments, an image of ZE3 VLP obtained though electron microscopy.

FIGS. 25A-25B depict, in accordance with certain embodiments, purification and partial purification of a recombinant immune complex with the zika soluble ectodomain E protein (amino acids 291-693, labeled ZsE in the figure) as the antigen linked to the antibody (ZEFL62 RIC). A sample of protein-G purified ZEFL62 RIC was analyzed on an SDS-PAGE gel stained with Coomassie (FIG. 25A). After sucrose gradient sedementation of the ZEFL62 VLP, a fraction was analyzed on an SDS-PAGE gel stained with Coomassie (FIG. 25B).

FIG. 26 depicts, in accordance with certain embodiment, IgG titers of mice after the second dose treatment with zika virus antigen. BALB/c mice (6 per group) were immunized subcutaneously with ZE3 N-terminal RIC, ZE3 C-terminal RIC, HBche-ZE3 VLP (abbreviated HBche), ZEFL RIC, and ZEFL HBche-VLP either alone or in various combinations of RIC and VLP mixed 1:1. Two groups, HBche-ZE3 alone and the HBche-ZE3+C-RIC, were not given alum as an adjuvant in order to test the effect of an adjuvant on the antibody titers elicited by the experimental groups. Except for the PBS control group, each dose delivered 4 μg total ZE3. The dose for the ZEFL-containing groups delivered 4 μg of ZEFL. Blood samples, collected after the second dose, were analyzed for ZIKV-specific antibodies by endpoint titer ELISA. The y-axis shows the geometric mean titers (GMT). Combination groups of the ZE3 VLP and RIC, delivered with or with alum, had higher antibody titers (approximately a 14-fold difference) than the HBche-ZE3 VLP delivered with or without adjuvant. Abbreviations: HBche, HBche-ZE3 and—Alum, without alum. Non-parametric Mann-Whitney test was used to evaluate significance of the differences indicated; p<0.05.

FIG. 27 depicts, in accordance with certain embodiments, IgG titers of mice after the third dose of treatment with zika virus antigen. Total anti-ZE3 IgG titers were measured by ELISA after the third dose. Geometric mean titers (GMT) were calculated for each group and are indicated by the horizontal line for each group, as well as indicated numerically in the table. Individual data points indicate the titer obtained with serum of each mouse. Non-parametric Mann-Whitney test was used to evaluate significance of the differences indicated; p<0.01.

FIG. 28 depicts, in accordance with certain embodiments, compares the total antibody titer (terminal bleed) of mice treated with a recombinant immune complex with the domain III of the zika virus E protein (K591-T696 of Accession No. AMC13911, labeled ZE3 in the figure) is linked to the antibody at the N-terminus (NRIC) or C-terminus (CRIC) of its heavy chain. Six Balb/C mice were given three doses of either ZDIII N-RIC or RIC over an 8-week period. Serum samples were collected and the antibody titers determined by ELISA. The terminal bleed serum samples were collected a little over a month after the third dose. The ELISA results showed that both the N-RIC and C-RIC produced comparable antibody titers.

FIG. 29 depicts, in accordance with certain embodiments, a plaque reduction neutralization test conducted with live zika virus. Similar neutralization activity was seen following immunization with either N-RIC or C-RIC.

FIG. 30 depicts, in accordance with certain embodiments, a vector map for a plant expression plasmid encoding the zika soluble ectodomain E protein (K591-T696 of Accession No. AMC13911) with a 6-His tag. The nucleic acid sequence of pBYRIleM-h6D8ZE3 is set forth in SEQ ID NO. 45.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As used herein, the term “virus-like particle” or “VLP” refers to multiple protein structure that mimic the organization and conformation of authentic native viruses but lack the viral genome. In some embodiments, expression of viral structural proteins, for example capsid or envelope proteins, result in the self-assembly of VLPs. In other embodiments, a viral core is required to facilitate the assembly of the VLP when fragments of a protein are the desired presentation targets at the surface of the VLP. Viral cores used in the design of VLPs include bacteriophage MS2, adeno-associated virus, adenovirus, and tobacco mosaic virus.

As used herein, the term “immune complex” refers to a complex comprising immunoglobulin molecules or fragments thereof bound to its cognate antigen. As used herein, the term “recombinant immune complex” or “RIC” refers to an immune complex that is not produced by the species that originally produces the immunoglobulin molecule in the immune complex. For example, an exemplary recombinant immune complex comprises human immunoglobulin but is synthesized by plants.

Developing effective, easily modifiable vaccine platforms is an important research focus since vaccination is considered to be one of the most effective ways to provide protection against infectious diseases. Both VLPs and RICs are easily modifiable vaccine platforms once a suitable antigen for generating an immune response against an infectious agent is identified. While VLPs and RICs have been shown to have great potential as a vaccine alone, the inventors surprisingly found that co-delivering VLPs with RICs produces a greater immune response than that can be obtained through delivering either alone at the same dose of the antigen.

In some aspects, combining RICs and VLPs potentiate their efficacy beyond the potential of either vaccine individually when used to vaccinate against many diseases. For example, as shown in Example 1, RICs and VLPs containing the human papillomavirus (HPV) minor capsid protein L2 (also referred to herein as “L2”) induced higher responses when the vaccine candidates were co-delivered at the same L2 dose. In some implementations, administration of a RIC and a VLP presenting the same virus protein produces a two- to three-fold increase in the immune response generated against the virus protein (for example, measure by titer of antibody against the virus protein) when compared to administration of RIC containing the same amount the virus protein alone or administration of VLP containing the same amount of the virus protein alone (see Example 2 concerning the matrix 2 protein of influenza virus). Higher antibody titers after each combinatorial dose could result in fewer total doses needed to achieve a protective response, which could improve vaccination rates due to a lesser reliance on patient compliance to return for booster doses. Further, fewer doses of a given vaccine would also lessen the economic cost of vaccinating the population, as fewer total vaccines will be needed to achieve a sufficient level of protection.

Accordingly, in certain aspects, the disclosure is directed to a universal vaccine platform comprising a VLP and at least one RIC, wherein the target antigen presented by the VLP and the target antigen in at least one RIC are from the same antigenic protein. In some aspects, the target antigen in the at least one RIC is a fragment of the target antigen in the VLP. In other aspects, the target antigen presented in the VLP and the target antigen in the RIC have the same amino acid sequence. Administration of the aforementioned vaccine composition to a mammalian subjects generates an immune response against the virus protein and thus the virus. In certain embodiments, the composition comprises the VLP and the at least one RIC at a ratio of 1:1. In some embodiments, universal vaccine platform comprises a VLP, a N-terminal RIC (or N-RIC), and a C-terminal RIC (or C-RIC). In some aspects, the target antigen in the N-RIC and the C-RIC are different portion of the same antigenic protein. In particular embodiments, compositions for generating an immune response against multiple types of HPV, multiple strains of influenza virus, and zika virus are described.

In some aspects of the universal vaccine platform, the VLP and the RIC do not contain identical fragments of the virus protein. For example, the fragment of the virus protein on the VLP is a different portion of the fragment of the virus protein in the RIC. In some aspects, the fragment of the virus protein on the VLP may overlap with a portion of the fragment of the virus protein in the RIC. In other aspects, the fragment of the virus protein on the VLP do not overlap with a portion of the fragment of the virus protein in the RIC. In still other aspects, the fragment of the virus protein on the VLP may be larger than the fragment of the virus protein in the RIC and comprise the same portion of the virus protein as the fragment of the virus protein in the RIC.

The flexibility of the VLP and the RIC allows for several antigens to be packaged into each platform while reaping the benefits of the vaccine synergy the platforms display. This is particularly beneficial, especially for flu vaccines, as the majority of the most promising universal influenza vaccines target more than one site on the influenza virion, for example, many recent vaccines pursue some combination of influenza virus surface proteins (Atsmon et al., 2012; Ellebedy et al., 2014; Ingle et al., 2017). Targeting multiple conserved regions of the influenza virion would aid in preventing influenza from escaping via a single mutation in one of its proteins; the fewer avenues of escape the better, especially when any of the virus' proteins could mutate. Indeed, despite its high conservation, even M2e has the potential to mutate, with the M2e of avian and swine influenza A featuring mutations at different points in the protein (Liu et al., 2005; Zhou et al., 2012). While consensus sequences are useful to a point, including multiple conserved, immunogenic antigens in the design of universal influenza vaccines would make the vaccine more effective for a longer period of time.

Even if the universal vaccine platform described herein is insufficient fully protect recipients after a single dose, the composition comprising the described VLP and RIC could be used as an adjuvant. For example, the composition for generating an immune response against multiple strains of influence virus could be administered as an adjuvant to existing flu vaccines to increase the efficacy of the existing vaccines from season to season. The immunogenicity of the RICs, VLPs, and the universal vaccine platform described herein can be enhanced through the use of glycoengineered plants to glycosylate the vaccines in favorable patterns (Shields et al., 2002; Maverakis et al., 2015)). Accordingly, in some aspects, the disclosure relates to methods of producing the described VLP and/or RIC in plants using a plant expression vector, for example, a geminivirus-based vector. The production of the vaccines in plants further compounds the reduction of the economic cost of the vaccine. In some implementations, the immunogenicity of the RICs, VLPs, and the universal vaccine platform described herein, even if they are produced by plant, may be further enhanced by co-administration with a vaccine adjuvant. Vaccine adjuvants commonly used with current vaccinations include, for example, alum (composed of aluminum salts), MF59 (an oil-in-water emulsion of squalene oil), ASO4 (a combination of alum and monophosphoryl lipid A), and ASO3 (an oil-in-water emulsion of α-tocopherol, squalene, and polysorbate 80).

### Virus-Like Particles

The VLPs described herein have a virus core formed from hepatitis B virus core antigen (HBcAg). Upon expression, HBcAg self-aggregate to form a VLP. The target antigen for inducing a desired immune response in a subject is linked to HBcAg and is presented upon VLP formation to an organism's immune system. In preferred embodiments, the target antigen is linked to HBcAg at its major insertion region (MIR), which is located at the tip of the α-helical spike. In some aspects, the target antigen is displayed on the surface of the VLP through the production of a fusion protein where the target antigen is inserted into the HBcAg protein between residues 77 and 78 of the HBcAg protein.

In some aspects, the VLPs are formed from coexpression of wildtype HBcAg proteins and HBcAg with the targeted antigen linked at its MIR to create mosaics. In other aspects, the VLPS are formed with a split core, where the HBcAg protein is expressed as distinct N- and C-terminal portions, which allows assembly of structural dimers even in the absence of covalent linkage. In yet other aspects, the VLPS are formed with a tandem core where two HBcAgs are joined together by a flexible linker to give a single fused dimer protein. In such embodiments, a target antigen may be linked to the MIR of just one of the HBcAgs or both. In some aspects, different target antigens may be linked to each of the MIRs in the tandem core.

In some embodiments, the VLPs described herein comprise two HBcAg monomers that are linked to form a HBcAg dimer, which self-aggregates to form the VLP (FIG. 2). In a preferred embodiment, at least one of the HBcAg monomers in the HBcAg dimer is linked to the target antigen at its MIR. In some aspects, the other HBcAg monomer in the HBcAg dimer does not have a target antigen linked to its MIR.

In certain embodiments of an expression cassette encoding a VLP disclosed herein, the target antigen with flanking linker regions is inserted into the tip of the α-helical spike of an HBc gene copy that is fused to another copy of HBc lacking the target antigen. In preferred embodiments, the linker regions are glycine serine linker sequences.

### Recombinant Immune Complexes

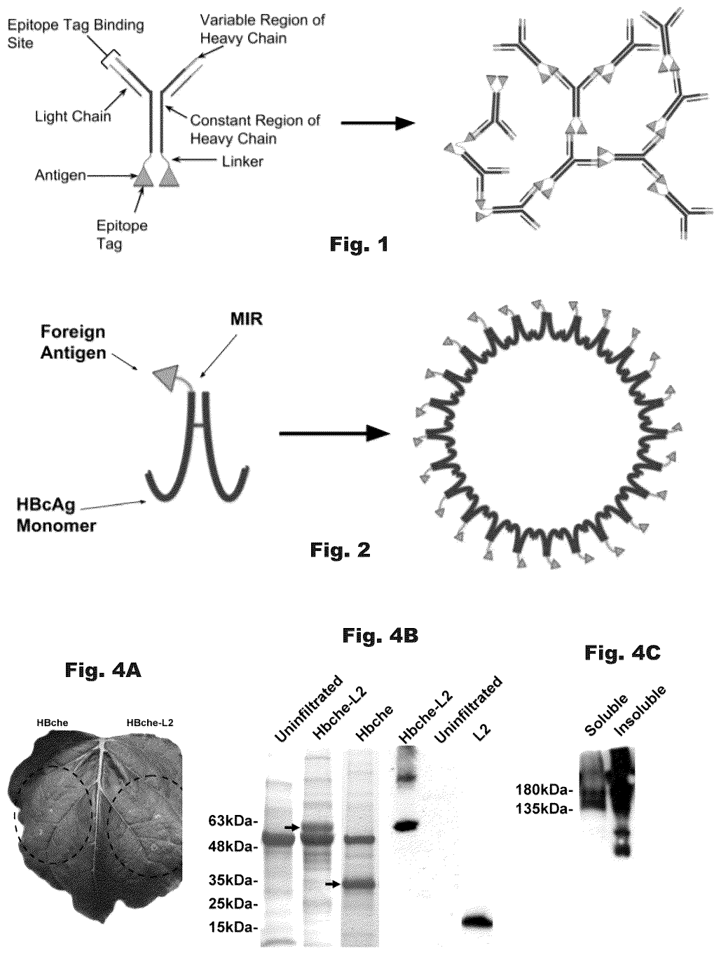

The RICs described herein comprise an immunoglobulin heavy chain, an epitope tag that can bind to the immunoglobulin heavy chain, and a target antigen. In some aspect, the immunoglobulin heavy chain is a camelid immunoglobulin. In certain embodiments, the RIC further comprises an immunoglobulin light chain. Thus, in some aspects, the RIC comprises a standard antibody (two heavy chains and two light chains joined to form a “Y” shaped molecule), an antigen, and an epitope tag that is recognized by the antibody (FIG. 1). The antibody binds to the epitope tags on other antibody fusions and forms a complex. In some embodiments, the RIC comprises human IgG 6D8, and the epitope tag is ebola glycoprotein epitope 6D8.

RICs described herein include conventional RICs where the target antigen is linked to the C-terminus of the immunoglobulin heavy chain and the epitope tag is linked to the other end of the target antigen (also referred to herein as “C-RIC”). The recombinant immune complex is produced by fusing a target antigen to the C-terminus of the heavy chain of an immunoglobulin that binds specifically to the antigen, wherein the co-expression of this fusion protein with the light chain of the antibody produces a fully formed immunoglobulin that is self-reactive, and results in the creation of an immune complex due to the bivalent binding capacity of the immunoglobulin. However, antigens with inaccessible N-termini cannot be easily used in the RIC platform without disrupting native antigenic conformation. Also described herein is a novel design of RIC where the target antigen is linked to the N-terminus of the immunoglobulin heavy chain and the epitope tag is linked to C-terminus of the immunoglobulin heavy chain (also referred to herein as “N-RIC”).

In certain embodiment of an expression vector encoding RICs, the expression vector comprises a expression cassette encoding the immunoglobulin heavy chain, the target antigen, and the epitope tag. In some aspects, the expression vector further comprises a second expression cassette encoding the immunoglobulin light chain.

### Human Papilloma Virus Vaccine Compositions

In some embodiments, the disclosure relates to vaccine compositions that target multiple subtypes of HPV and methods of generating an immune response in a mammalian subject against multiple subtypes of HPV.

Papillomaviruses are an ancient and diverse group of viruses, and over 200 subtypes currently known to infect humans (Doorbar et al. 2015). Diverse human papillomavirus (HPV) subtypes are responsible for considerable disease burden worldwide, necessitating safe, cheap, and effective vaccines. HPV is the most common pathogen sexually transmitted disease, with more than 15 HPV oncogenic types responsible for oropharyngeal and anogenital cancers that result in significant morbidity and mortality worldwide (Crow 2012). Currently available prophylactic HPV vaccines target the L1 capsid protein, which self-assembles into highly immunogenic VLPs (Kirnbauer et al. 1992). Because neutralizing epitopes found on L1 are not broadly conserved among HPV types, multiple L1 proteins must be included in vaccine preparations to protect against multiple HPV types. The most broadly protective vaccine approved to date, Garadasil-9, provides protection against HPV types 6, 11, 18, 31, 33, 45, 52, 58. However cross-protection with other HPV types is minimal, and the complex formulation of the vaccine makes it cost-prohibitive for much of the world (Brown et al. 2009; Wheeler et al. 2009; Mariani and Venuti 2010; Vesikari et al. 2015).

The HPV minor capsid protein L2 is a promising candidate to create broadly protective HPV vaccines, though it is poorly immunogenic by itself. Unlike L1, neutralizing epitopes on the N-terminus of L2 are broadly conserved, and L2 antibodies can provide protection against multiple HPV subtypes (Kondo et al. 2007; Gambhira et al. 2007b; Alphs et al. 2008; Schellenbacher et al. 2017). However, as L2 is unable to form VLPs, it is poorly immunogenic by itself, necessitating strategies to enhance L2 antibody production. Accordingly, there is a need for improved HPV vaccine designs using L2.

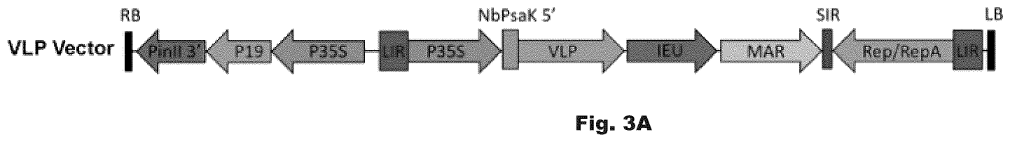

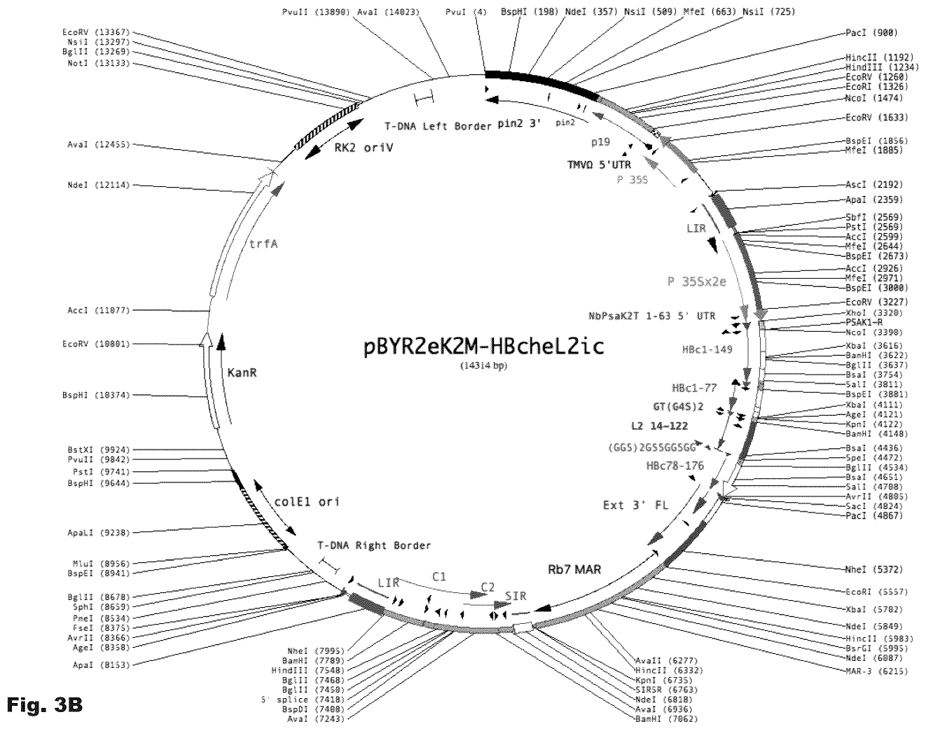

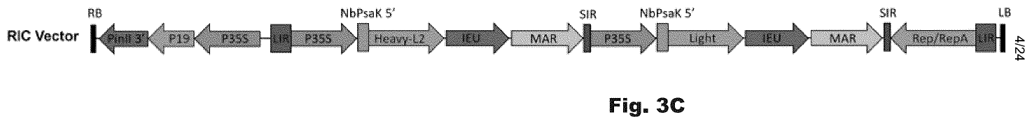

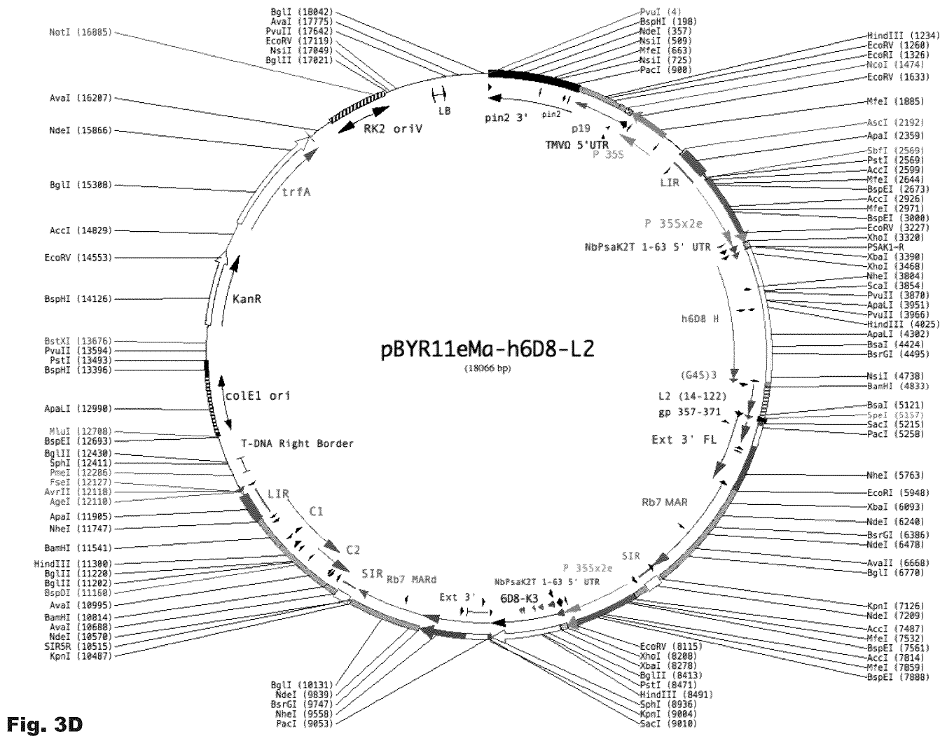

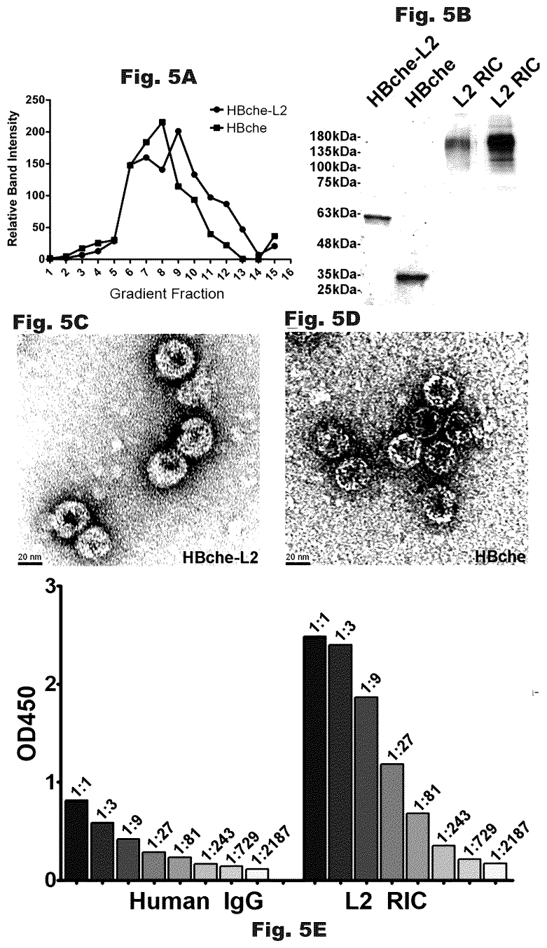

A successful vaccine based on HPV minor capsid protein L2 has yet to be confirmed. However, as shown in FIGS. 3 and 4, the VLP and RIC based on HPV minor capsid protein L2 described herein, when administered alone or together generates an immune response in the subject against HPV minor capsid protein L2. Specifically, the immune response generated by the administration of the described VLP and/or RIC based on HPV minor capsid protein L2, reduce infectivity of HPV16 (FIG. 5). When the described VLP is administered with the described RIC, a synergistic increase in the immune response produced against HPV minor capsid protein L2 takes place. Accordingly, the disclosure relates a VLP based on HPV minor capsid protein L2, a RIC based on HPV minor capsid protein L2, related vaccine compositions for target multiple subtypes of HPV, and methods of generating an immune response against multiple subtypes of HPV.

As referenced herein, the term “fragments of HPV minor capsid protein L2” refers to fragments from the highly conserved N-terminal region of the minor capsid protein L2 of human papillomaviruses. In certain embodiments, the high conserved N-terminal region of the HPV minor capsid protein L2 refers to an amino sequence corresponding to the first 200 amino acid residues of the L2 protein based on the amino acid sequence of HPV16 minor capsid protein L2, for example, residues 14-120 or residues 14-122. In some aspects, the amino acid sequence of the highly conserved N-terminal region of the L2 protein is the sequence set forth in Gene Accession CAC51368.1. As the N-terminus of the L2 protein is highly conserved across HPVs, the amino acid sequence of the highly conserved N-terminal region of the L2 protein may refer to the corresponding region in other HPVs. In some aspects, the corresponding nucleic acid sequence encoding the high conserved N-terminal region of the HPV minor capsid protein L2 is set forth in residues 1-473 of GenBank Accession No. KC330735. In other aspects, the corresponding nucleic acid sequence encoding the high conserved N-terminal region of the HPV minor capsid protein L2 may be a functionally equivalent version of nucleic acids 1-473 of GenBank Accession No. KC330735, where the translated product of the nucleic acid sequence has an amino acid sequence with at least 55%, at least 60%, at least 75%, at least 80%, at least 85%, at least 90%, at least 95%, at least 97%, or at least 99% identity to the first 200 amino acid of the sequence set forth in Gene Accession CAC51368.1.

The amino acid positions of HPV minor capsid protein L2 referenced herein are based on the amino acid sequence of HPV16 minor capsid protein L2 (Accession No. AGH32604.1). Due to the high level of conservation, the immune response generated from RIC and VLP targeting HPV minor capsid protein L2, even if the antigenic fragments are based on the amino acid sequence of HPV16 minor capsid protein L2 would also be an immune response that targets a variety of HPV subtypes aside from HPV16.

In certain embodiments, the vaccine composition comprises a VLP assembled with a fragment of HPV minor capsid protein L2 selected from the first 200 amino acid residues from the N-terminus of HPV minor capsid protein L2 and a recombinant immune complex (RIC) comprising, as antigenic portion, an amino acid sequence of at least 8 continuous amino acids from the first 200 amino acid residues from the N-terminus of HPV minor capsid protein L2.

In certain implementations of the method of generating an immune response in a mammalian subject against HPV, which are also methods of increasing the immunogenicity of HPV minor capsid protein L2, the method comprises administering to the mammalian subject a RIC comprising an HPV minor capsid protein L2 antigenic fragment, wherein the HPV minor capsid protein L2 antigenic fragment comprises an amino acid sequence of at least 8 continuous amino acids from the first 200 amino acid residues from the N-terminus of HPV minor capsid protein L2. In some implementations, the RIC is administered with a VLP displaying a fragment of HPV minor capsid protein L2. In some aspects, the VLP comprises a hepatis B virus core, for example the HBcAg protein. In certain implementations, the VLP and the RIC are administered to the mammalian subject in two vaccination events, wherein each vaccination event comprises administration of one dose of the VLP and one dose of the RIC. In some aspects, the two vaccination events are separated by a period of at least 14 day, at least 21 days, at least 28 days, at least 35 days, at least 42 days, at least 49 days, at least 56 days, about three weeks, about four weeks, about five week, about six weeks, about seven weeks, or about eight weeks. As used herein, the term “about” refers to ±3 days, ±2 days, or ±1 day. The methods of administering the VLP and RIC may be any established methods of vaccination in the prior art. In some instances, the VLP and RIC are administered subcutaneously. In some implementations, the VLP and RIC are administered with an adjuvant, for example, Imject® Alum (Thermo Scientific, Rockford, TL).

a. Virus-Like Particle with HPV Minor Capsid Protein L2

The VLPs of the disclosure include embodiments where fragments of HPV minor capsid protein L2 displayed on the surface of viral core, for example a hepatitis B core (HBc). In some aspects, the VLPs of the disclosure also refer to fragments of HPV minor capsid protein L2 fused to fragments of HPV major capsid protein L i, which can self-assemble into a VLP. In certain embodiments, the VLPs have a core comprising the HBcAg protein and the fragment of HPV minor capsid protein L2 is displayed on the surface of HBc VLP. In some implementations, the fragment of HPV minor capsid protein L2 is displayed on the surface of HBc VLP through the production of a fusion protein where the HPV fragment is insert into the HBcAg protein between residues 77 and 78 of the HBcAg protein. In some embodiments, the fragment of HPV minor capsid protein L2 with flanking linker regions is inserted into the tip of the α-helical spike of an HBc gene copy that is fused to another copy of HBc lacking the L2 insert.

As the N-terminus of HPV minor capsid L2 protein is known to contain cross-neutralizing epitopes, the VLPs of the disclosure display fragments of HPV minor capsid protein L2 comprising at least 100 continuous amino acid residues from the first 200 amino acid residues from the N-terminus of the HPV minor capsid protein L2. In some implementations, the VLPs display at least 100 continuous amino acid residues from the amino acid residues 11-200 of the HPV16 minor capsid protein L2, for example, amino acid residues 11-128, amino acid residues 14-120, or amino acid residues 14-122 of HPV16 minor capsid protein L2. In certain embodiments, the fragments of HPV minor capsid protein L2 comprise about 105, 106, 107, 108, 109, 110, 120, 130, 140, 150, 160, 170, 180, 190, 200, 210, 220, 230, or 240 amino acid residues. In some aspects, the fragments of HPV minor capsid protein L2 is a string of several epitopes of HPV minor capsid protein L2.

In some embodiments of an expression cassette for producing the HPV VLP, the expression cassette comprises a DNA sequence encoding amino acid residues 1-149 of HBcAg, a linker (G₂S)₅G, amino acid residues 1-77 of HBcAg, a linker GT(G₄S)₂, amino acid residues 14-122 of HPV16 minor capsid protein L2, a linker (GGS)₂GSSGGSGG, and amino acid residues 78-176 of HBcAg.

The HBc VLPs are potently immunogenic in mice, generating very consistent and high antibody titers directed against HPV L2 (>1,000,000) (FIG. 6), which is as high as those seen with L1 vaccines.

b. Recombinant Immune Complex with HPV Minor Capsid Protein L2

The HPV RIC comprises an immunoglobulin heavy chain and a fragment of HPV minor capsid protein L2 wherein the fragment of HPV minor capsid protein L2 is genetically fused to the immunoglobulin heavy chain. In some embodiments, the HPV RIC is a C—RIC or N-RIC.

In some embodiments, the fragment of HPV minor capsid protein L2 is inserted into the gene encoding humanized mAb 6D8 heavy chain, resulting in 6D8 epitope-tagged fragment of HPV minor capsid protein L2. Accordingly, the RIC further comprises an ebola antigenic fragment, in particular, the GP1 protein. In one aspect, the humanized mAb 6D8 heavy chain is produced from substituting the H2 chain of mouse monoclonal antibody 6D8 with the human constant region sequences for gamma type 1 chain.

In some embodiments, the antigenic fragment of HPV minor capsid protein L2 comprises an amino acid sequence of at least 8, at least 9, at least 10, at least 11, at least 12, at least 15, at least 20, at least 25, at least 30, at least 40, at least 50, at least 60, at least 70, at least 80, at least 90, at least 100, or at least 110 continuous amino acids from the first 200 amino acid residues from the N-terminus of HPV minor capsid protein L2.

In some embodiments, the RIC comprises at least one peptide sequence from HPV minor capsid protein L2 selected from the group consisting of: amino acid residues 17-31, amino acid residues 17-36, amino acid residues 56-75, amino acid residues 65-85, and amino acid residues 96-115. In one embodiment, the fragment of HPV minor capsid protein L2 genetically fused to an immunoglobulin has an amino acid sequence consisting of amino acid residues 14-122. The RIC is potently immunogenic in mice, generating very consistent and high antibody titers directed against HPV L2 (>1,000,000).

c. Methods of Production

A plant-based transient expression system based on bean yellow dwarf virus was designed to produce the VLPs and RICs of the disclosure (see example vector in FIG. 3). In this system, the viral replication machinery is used to amplify the target gene to high copy number in the plant nucleus (Huang et al. 2009). After rigorous genetic optimization, the system is capable of producing very high levels of recombinant protein, up to 30-50% of the total soluble plant protein, in 4-5 days (Diamos et al. 2016). Using this system, milligram quantities of fully assembled and potently immunogenic HBc VLPs displaying fragments of HPV minor capsid protein L2 can be produced and purified through a simple one-step purification process (FIGS. 4A-4C and 6). Remarkably, all of the VLPs used for immunization in the Examples were derived from a single plant leaf, demonstrating the promise of plant-based recombinant expression systems.

### Influenza Virus

In some embodiments, the disclosure relates to vaccine compositions that target multiple strains of the influenza virus (also referred to herein as “flu virus”) and methods of generating an immune response in a mammalian subject against multiple strains of the flu virus.

Influenza is a deadly disease that poses a major threat to global health. Influenza is a historically and socially significant disease that is characterized by the sudden onset of a high fever, runny nose, fatigue, muscle pains, sneezing, coughing, and general malaise (World Health Organization, 2018). In a meta-analysis of six influenza seasons in the United States (from the 2010-2011 season to the 2015-2016 season), it was estimated that the number of influenza-related illnesses ranged from 9.2 million to 35.6 million per season, including 140,000 to 710,000 influenza-related hospitalizations (Rolfes et al., 2018). Further, the disease is also estimated to cost the U.S. economy anywhere from $6.3 to $25.3 billion annually (Putri et al., 2018). On a global scale, influenza-associated mortality typically ranges from 291,243 to 645,832 influenza-associated respiratory deaths per year (Juliano et al., 2018), with most (>99%) deaths of children below the age of 5 years old occurring in developing countries. However, should a pandemic strain of influenza similar in severity to the 1918 Spanish Flu pandemic occur, the cost to human life could be catastrophic, which would simultaneously cripple the global economy; the World Bank estimates that such a pandemic would cost the global economy approximately 5% of its GDP in the pandemic's first year alone (Burns et al., 2006).

Due to the disease's impact around the world, there have been calls for substantial improvement in the methods used to estimate the global burden of influenza (e.g. including data on nonrespiratory complications of influenza infection, severe influenza case data, etc.) (Bresse et al., 2018), indicating that not only a potentially substantial number of cases are currently left unreported and unaccounted for within the literature, but that influenza may be a larger problem than is currently believed. While influenza vaccines lessen the annual impact of influenza on the world, it is of the utmost importance that vaccines with high rates of efficacy are developed in a timely manner.

Influenza is a member of the Orthomyxoviridae family of viruses, a group of single-stranded negative sense RNA viruses, and its genome is comprised of eight segments (McGeoch et al., 1976). There are four types influenza viruses: A, B, C, and D; though influenza C is not commonly associated with disease in most populations, being observed only in isolated, sporadic outbreaks in children younger than the age of six years old and elderly populations (Matsuzaki et al., 2006; Smith et al., 2016). Influenza D has been shown to only infect swine and cattle (Hause et al., 2014) (Smith et al., 2016). Influenza A and B, meanwhile, regularly circulate and cause disease in humans, with rates of influenza-related hospitalizations being higher in seasons where influenza A viruses dominate (Thompson et al., 2004). Influenza A is also more routinely the cause of epidemics and pandemics (Hay et al., 2001), as influenza A viruses have a higher mutation rate than influenza B viruses (Nobusawa & Sato, 2006) and consequently have a higher propensity to evade the protective immune response that influenza vaccines or infections confer. The high mutation rate of influenza A viruses, when compared to influenza B viruses, is partially due to influenza B viruses generally being limited to infecting humans (Hay et al., 2001). Meanwhile, influenza A viruses are able to infect a range of creatures, including, but not limited to, pigs, birds, and humans (Hay et al., 2001), increasing the likelihood of antigenic shift between human and zoonotic strains. Influenza A's association with high levels of hospitalization, seasonal epidemics, and global pandemics makes the need for a ‘universal’ influenza A vaccine that maintains its efficacy and protection from season to season, despite the virus' high rate of mutation, absolutely essential to preventing the influenza pandemics of the future.

The surface proteins of influenza A, the type most often associated with epidemics and pandemics, mutate at a very high frequency from season to season, reducing the efficacy of seasonal influenza vaccines. Over six influenza seasons in the U.S., from 2010 to 2016, it was determined that overall vaccination rates ranged from 42%-47% of the population, preventing anywhere from 1.6 million to 6.7 million illnesses, 790,000-3.1 million outpatient medical visits, 39,000-87,000 hospitalizations, and 3,000-10,000 influenza-related deaths (Rolfes et al., 2018). However, seasonal influenza vaccines are routinely associated with low rates of vaccine efficacy (VE); the U.S. Centers for Disease Control and Prevention (CDC) reported VEs of 56% for the 2012-2013 (Jackson et al., 2013), 61% for the 2013-2014 season (Flannery et al., 2014), 23% for the 2014-2015 season (Flannery et al., 2015), 48% for the 2015-2016 season (Jackson et al., 2017), and 48% for the 2016-2017 season (Flannery et al., 2017). Furthermore, during the 2017/2018 influenza season, VE against the circulating strain of influenza A (H3N2) was estimated to be as low as 25% in the United States (Centers for Disease Control and Prevention, 2018), 17% in Canada (Skowronski et al., 2018) and 10% in Australia (Sullivan et al., 2017) despite the 2017/2018 influenza vaccine containing influenza of the same subtype and clade. This was due, in part, to three mutations in hemagglutinin (HA), a protein on the influenza virus' surface.

The vulnerability of influenza vaccines to small mutations like those observed in the 2017/2018 strain's HA protein is due primarily to the vaccines' composition, which involve including three to four strains of inactivated or attenuated influenza virus. Predictions, and subsequent recommendations, are made by the scientific community on an annual basis as to which three to four strains will most likely be in circulation during that year's influenza season. Then, vaccines composed of the predicted strains are mass-produced, traditionally in eggs, and shipped before the influenza season starts. Due to the structure and behavior of the influenza virus, traditional methods of influenza vaccine production, as well as vaccine composition, it comes as no surprise that the influenza virus is regularly able to mutate in ways that reduce the VE of a given season's influenza vaccine.

The influenza A genome encodes at least ten proteins and up to 14 proteins through strain-dependent alternative splicing (Eisfeld et al., 2015; Suarez et al., 2016). The ten common influenza A proteins can be grouped into the surface proteins, which include hemagglutinin (HA), neuraminidase (NA), and the matrix 2 protein (M2); the internal proteins, which include the nucleoprotein (NP), the matrix 1 protein (M1), polymerase basic protein 1 (PB1), polymerase basic protein 2 (PB2), and polymerase acidic protein (PA); and the non-structural proteins 1 (NS1) and 2 (NS2) (Suarez et al., 2016). Segment 1 encodes PB2, segment 2 encodes PB1, segment 3 encodes PA, segment 4 encodes HA, segment 5 encodes NP, segment 6 encodes NA, segment 7 encodes both M1 and M2, and segment 8 encodes NS1 and NS2 (Inglis et al., 1976). HA and NA, in particular, are common targets of recombinant influenza vaccines, as HA's primary role is to facilitate viral entry into target cells through binding to sialic acid-containing receptors on the host cell (Skehel & Wiley, 2000) and NA promotes the release of newly-formed influenza virions through the removal of sialic acid residues on both the host cell and the nascent virion (Mitnaul et al., 2000). However, while the neutralization of either could provide protection against influenza A infection, HA and NA mutate frequently from season to season (Webster et al., 1982). This has led to the search for conserved, protective epitopes in not only HA (Kramer & Palese, 2019) and NA (Kosik et al., 2019), but also other influenza A proteins, so that a ‘universal’ influenza vaccine that is effective from season to season can be developed.

Certain regions of these proteins are conserved between strains of influenza A, making them attractive targets for the development of a ‘universal’ influenza vaccine. One of these regions can be found on influenza matrix 2 protein M2, which is a tetrameric integral membrane protein that, despite being found at low levels on influenza A virion, facilitates viral uncoating in its role as a proton channel (Lamb et al., 1985). The ectodomain of the influenza matrix 2 protein (M2e) has not changed significantly since it was first identified in 1933 (Fiers et al., 2004). This highly-conserved region of M2 is poorly immunogenic on its own, but when conjugated or fused to potent adjuvants or carriers, it becomes a potent target against influenza A (Mardanova & Ravin, 2018). M2 is expressed on the surface of infected cells at nearly the same rate as NA, but is incorporated into virions much less than NA, with only 14 to 68 molecules of M2 per virion versus 198-211 molecules of NA, suggesting that M2 is selectively excluded from forming virions (Lamb et al., 1985; Zebedee et al., 1988). Despite this, vaccines targeting M2e have demonstrated protection in several studies, with this protection having been determined to be due not to the prevention of infection, but instead through Fc-receptor dependent antibody-dependent cell cytotoxicity (ADCC) and alveolar macrophage antibody-dependent cell-mediated phagocytosis (ADCP) of infected cells (El Bakkouri et al., 2011). Additionally, it has been discovered that lung-resident Thl7 CD4 T cells specific for M2e tetramers are broadly effective against influenza infection, indicating that the anti-M2e response is not limited only to antibody-dependent responses (Eliasson et al., 2018).

In clinical trials, vaccines targeting M2e have been well-tolerated, with studies investigating M2e expressed recombinantly on hepatitis B core antigen (HBc) (Fiers et al., 2009), even in the presence of anti-HBc antibodies, and fused to flagellin (Turley et al., 2011) demonstrating safety and efficacy. Several other clinical trials have been conducted around the world investigating M2e's potential as a vaccine antigen (Scorza et al., 2016) to varying degrees of success. However, to the best of our knowledge, no studies have attempted to express M2e recombinantly in a recombinant immune complex (RIC), a promising ‘universal vaccine platform’ that could boost the immunogenicity of M2e substantially and whose modularity could allow for the addition of other prominent and conserved influenza targets and adjuvants to adjust to whatever potential hurdles the influenza epidemics and pandemics of the future may have to offer.

In certain implementations of the method of generating an immune response in a mammalian subject against influenza virus, which are also methods of increasing the immunogenicity of M2e protein of influenza A virus, the method comprises administering to the mammalian subject a VLP presenting M2e. In some implementations, a RIC comprising M2e is also administered. In some aspects, RIC comprising M2e and VLP presenting M2e are co-administered. In some aspects, the RIC and the VLP are coadministered in a ratio of 1:1. In certain implementations, the VLP and the RIC are administered to the mammalian subject in three vaccination events, wherein each vaccination event comprises administration of one dose of the VLP and/or one dose of the RIC. In some aspects, two vaccination events are separated by a period of at least 14 day, at least 21 days, at least 28 days, at least 35 days, at least 42 days, at least 49 days, at least 56 days, about three weeks, about four weeks, about five week, about six weeks, about seven weeks, or about eight weeks. As used herein, the term “about” refers to ±3 days, ±2 days, or 1 day. The methods of administering the VLP and RIC may be any established methods of vaccination in the prior art. In some instances, the VLP and RIC are administered subcutaneously. In some implementations, the VLP and RIC are administered with an adjuvant.

As shown in Example 2 section d, recipients of the VLP+RIC exhibited endpoint anti-M2e antibody titers that were 2 to 3 times higher than mice that received the VLP alone. While IgG2a:IgG1 ratios were higher in mice vaccinated solely with the VLP, the higher overall titers are encouraging and demonstrate a degree of interaction between the RIC and VLP vaccines. Thus, the VLP presenting M2e and RICs comprising M2e are promising new universal influenza A vaccines. Additionally, co-delivering different types of recombinant vaccines could reduce the total number of vaccine doses needed to achieve a protective immune response.

a. Virus-Like Particle with M2e

The VLPs disclosed herein for generating an immune response against influenza viruses present M2e protein. In certain embodiments, the amino acid sequence of the M2e protein presented in the VLP is SLLTEVETPIRNEWGCRCNDSSD (SEQ ID NO. 9). In preferred embodiments, the VLPs present a dimeric M2e protein, where two monomeric M2e proteins are linked with a linker sequence. In some aspects, the linker is a glycine serine linker sequence. In particular embodiments, the amino acid sequence of dimeric M2e protein presented by the VLPs is set forth in SEQ ID NO. 10.

b. Recombinant Immune Complex with M2e

The influenza virus RIC comprises an immunoglobulin heavy chain and M2e wherein the M2e is genetically fused to the immunoglobulin heavy chain. In certain embodiments, the amino acid sequence of the M2e protein presented in the RIC is SLLTEVETPIRNEWGCRCNDSSD (SEQ ID NO. 9). In certain embodiments, the influenza virus RIC comprises an immunoglobulin heavy chain linked to a M2e dimer. The M2e dimer is formed from a glycine serine linkage sequence linking two M2e proteins. In some embodiments, the influenza virus RIC is a C—RIC or N-RIC.

c. Methods of Production

It has been shown that M2e and M2e-containing vaccines has been able to be expressed effectively in plants (Nemchinov & Natilla, 2007). RICs comprising M2e and VLPs presenting M2e can be produced in plants using a geminiviral expression vector (see SEQ ID NOs. 19 and 20), and the VLPs and RICs can be extracted and purified from plants accordingly to methods established in the art.

### Zika Virus Vaccine Compositions

In some embodiments, the disclosure relates to vaccine compositions that target zika virus and methods of generating an immune response in a mammalian subject against zika virus.

Zika virus (ZIKV) is a positive-sense single-stranded RNA virus that is a part of the genus Flavivirus and family Flaviviridae (Oliveira et al., 2017). Currently, the genus Flavivirus consists of fifty-three documented species along with a growing number of tentative species (Sim6n et al., 2017). These viruses produce a single polyprotein that is later cleaved into three structural proteins (C, prM and E) and seven nonstructural proteins (Oliveira et al., 2017). The prM (precursor transmembrane M) protein is proteolytically cleaved during virion maturation by a host cell protease to create the membrane (M) and pr protein. On a mature virus particle, a hundred and eighty copies of the envelope glycoprotein (E) and membrane (M) proteins can be found arranged in an icosahedral structure with 90 E dimers. This structure covers the viral surface (Boigard et al., 2017; Dai et al., 2016).

ZIKV is considered a global public health threat due to factors involving its spread and involvement with neonatal complications. From 2015-2017, Zika viral transmission has been reported in over 69 countries worldwide. In February 2016, the World Health Organization declared a Public Health Emergency of International Concern in response to the growing number of global Zika infections and the increasing amount of evidence suggesting links between Zika infection and congenital/neurological complications such as Guillain-Barre Syndrome and neonatal microcephaly (Rabaan et al., 2017; Wilder-Smith et al., 2018). Since then, there has been significant interest in developing vaccines and other therapeutic aids against the Zika virus. At this time, there are 45 vaccine candidates that were tested in non-clinical studies. Of the vaccine candidates that advanced past animal pre-clinical studies, several are in phase 1 human clinical trials and at least one is in phase 2 clinical trials ((NIAID); Durbin and Wilder-Smith, 2017; Wilder-Smith et al., 2018).

The main antigenic determinant of the virus is the envelope glycoprotein (E) since it is available on the surface of the mature virus particle and can be targeted by a number of neutralizing antibodies (Yang et al., 2018; Zhang et al., 2017). For this reason, many vaccine candidates utilize the ZIKV E protein ((NIAID)). One example is the experimental vaccine candidate currently in phase 2 clinical trials. This DNA vaccine candidate encodes the ZIKV wild type precursor transmembrane M (prM) and envelope (E) protein ((NIAID)). However, as of now, DNA vaccines are not licensed for human use and may have some risk of chromosomal integration via nonhomologous recombination (Barzon and Palu, 2017). Plant-produced, vaccines can potentially overcome safety and cost concerns associated with other ZIKV vaccine candidates, including inactivated virus, mRNA or DNA-based vaccines, and adenovirus-vectored vaccines. Plant expression systems are highly scalable and avoid many of the costs of traditional systems, such as expensive bioreactors, thereby allowing cheaper production of biological products (Alam et al., 2018; Tuse et al., 2014). Additionally, using a recombinant protein vaccine also removes the safety concerns of improperly inactivated virus, genomic insertion, and the development of immune responses to adenoviral vectors (Yang et al., 2018).

The E protein contains three structurally separate domains (Zhang et al., 2017). Of these domains, the E domain III (ZE3) is a promising target for vaccine development since it has been shown to contain a number of epitopes for neutralizing, type-specific monoclonal antibodies (Dai et al., 2016; Haiyan Zhao et al., 2016; Yang et al., 2017). Since neutralizing antibodies developed against approved vaccines for yellow fever virus and tick-borne encephalitis virus, both of which are closely related to ZIKV, appear to have a correlation with viral protection (Belmusto-Worn et al., 2005; Heinz et al., 2007), ZE3 is an important target. Furthermore, ZE3-specific antibodies do not show dengue virus antibody dependent enhancement (Stettler et al., 2016). Antibody-dependent enhancement occurs when non-neutralizing antibodies developed in response to one viral infection cross-reacts and forms complexes with another virus upon infection. These complexes bind to cells with Fc-gamma or complement-associated receptors and are taken up by myeloid cells. However, since the antibodies merely bind to and do not neutralize the virus, the severity of viral infection is enhanced (Taylor et al., 2015). Published work utilizing a subunit ZE3 protein vaccine candidate showed an absence of antibody dependent enhancement of dengue viral infection (Yang et al., 2017). This result, along with the presence of known, neutralizing antibody epitopes on ZE3, render this antigen a prime target for vaccination.

In certain implementations of the method of generating an immune response in a mammalian subject against zika virus, the method comprises administering to the mammalian subject a VLP presenting a zika virus antigen. In some implementations, a RIC comprising a zika virus antigen is also administered. In some aspects, RIC comprising a zika virus antigen and VLP presenting a zika virus antigen are co-administered. In some aspects, the RIC and the VLP are coadministered in a ratio of 1:1. In certain implementations, the VLP and the RIC are administered to the mammalian subject in three vaccination events, wherein each vaccination event comprises administration of one dose of the VLP and/or one dose of the RIC. In some implementations, the three vaccination events are performed within a period of 8 weeks. In some aspects, two vaccination events are separated by a period of at least 14 day, at least 21 days, at least 28 days, at least 35 days, at least 42 days, at least 49 days, at least 56 days, about three weeks, about four weeks, about five week, about six weeks, about seven weeks, or about eight weeks. As used herein, the term “about” refers to ±3 days, ±2 days, or ±1 day. The methods of administering the VLP and RIC may be any established methods of vaccination in the prior art. In some instances, the VLP and RIC are administered subcutaneously. In some implementations, the VLP and RIC are administered with an adjuvant.

a. Virus-Like Particle with Zika Virus Antigens

The VLPs disclosed herein for generating an immune response against zika viruses present an antigen selected from the group consisting of zika virus E protein domain 3 protein (ZE3), zika virus fusion loop antigen (ZE), and zika virus soluble envelope protein (Zse). In some aspects, the amino acid sequence of ZE3 is set forth in residues 591-696 of GenBank Accession No. AMC13911. In some aspects, the amino acid sequence of ZE is set forth in residues 352-412 of GenBank Accession No. AMC13911. In some aspects, the amino acid sequence of Zse is set forth in residues 291-693 of GenBank Accession No. AMC13911. In other aspects, the amino acid sequences of ZE3, ZE, and Zse may be functionally equivalent versions of corresponding regions of GenBank Accession No. AMC13911 from other strains of zika virus, for example, the corresponding sequences of ZE3, ZE, Zse in GenBank Accession Nos. AY632535, KU321639, KJ776791, KF383115, KF383116, KF383117, KF383118, KF383119, KF268948, KF268949, KF268950, EU545988, KF993678, JN860885, HQ234499, KU501215, KU501216, KU501217.

The zika virus VLPs can be produced in plants using a geminiviral expression vector (see SEQ ID NOs. 34 and 35), and the VLPs can be extracted and purified from plants accordingly to methods established in the art.

b. Recombinant Immune Complex with Zika Virus Antigens

The zika virus RIC comprises an immunoglobulin heavy chain and a zika virus antigen genetically fused to the immunoglobulin heavy chain. In some embodiments, the zika virus RIC is a C—RIC or N-RIC. The zika virus antigen is selected from the group consisting of zika virus E protein domain 3 protein (ZE3), zika virus fusion loop antigen (ZE), and zika virus soluble envelope protein (Zse). In some aspects, the amino acid sequence of ZE3 is set forth in residues 591-696 of GenBank Accession No. AMC13911. In some aspects, the amino acid sequence of ZE is set forth in residues 352-412 of GenBank Accession No. AMC13911. In some aspects, the amino acid sequence of Zse is set forth in residues 291-693 of GenBank Accession No. AMC13911. In other aspects, the amino acid sequences of ZE3, ZE, and Zse may be functionally equivalent versions of corresponding regions of GenBank Accession No. AMC13911 from other strains of zika virus, for example, the corresponding sequences of ZE3, ZE, Zse in GenBank Accession Nos. AY632535, KU321639, KJ776791, KF383115, KF383116, KF383117, KF383118, KF383119, KF268948, KF268949, KF268950, EU545988, KF993678, JN860885, HQ234499, KU501215, KU501216, KU501217.

The zika virus RICs can be produced in plants using a geminiviral expression vector (see SEQ ID NOs. 36-38), and the RICs can be extracted and purified from plants accordingly to methods established in the art.

### ILLUSTRATIVE, NON-LIMITING EXAMPLE IN ACCORDANCE WITH CERTAIN EMBODIMENTS

The disclosure is further illustrated by the following examples that should not be construed as limiting. The contents of all references, patents, and published patent applications cited throughout this application, as well as the Figures, are incorporated herein by reference in their entirety for all purposes.

### Example 1. Human Papillomavirus Vaccine Compositions

a. Materials and Methods
i. Vector Construction

### 1. Virus-Like Particle

As most broadly neutralizing HPV antibodies are derived from the highly conserved N-terminal region of L2, amino acids 14-122 of HPV16 L2 were used to create HBc VLPs. L2 with flanking linker regions was inserted into the tip of the α-helical spike of an HBc gene copy which was fused to another copy of HBc lacking the L2 insert. This arrangement allows the formation of HBc dimers that contain only a single copy of L2, increasing VLP stability (Peyret et al. 2015). This heterodimer is referred to as HBche-L2. A dicot plant-optimized HPV16 L2 coding sequence was designed based upon the sequence of GenBank Accession No. CAC51368.1 and synthesized in vitro using synthetic oligonucleotides by the method described (Stemmer et al., 1995). The plant-optimized L2 nucleotide sequence encoding residues 1-473 is posted at GenBank Accession No. KC330735. PCR end-tailoring was used to insert XbaI and SpeI sites flanking the L2 aa 14-122 using primers L2-14-Xba-F (SEQ ID NO. 1: CGTCTAGAGTCCGCAACCCAACTTTACAAG) and L2-122-Spe-R (SEQ ID NO. 2: G GGACTAGTTGGGGCACCAGCATC). The SpeI site was fused to a sequence encoding a 6His tag, and the resulting fusion was cloned into a geminiviral replicon vector (Diamos, 2016) to produce pBYe3R2K2Mc-L2(14-122)6H.

The HBche heterodimer VLP system was adapted from Peyret et al (2015). Using the plant optimized HBc gene (Huang et al., 2009), inventors constructed a DNA sequence encoding a dimer comprising HBc aa 1-149, a linker (G₂S)₅G (SEQ ID NO. 39), HBc aa 1-77, a linker GT(G₄S)₂ (SEQ ID NO. 40), HIPV-16 L2 aa 14-122, a linker (GGS)₂GSSGGSGG (SEQ ID NO. 41), and HBc aa 78-176. The dimer sequence was generated using multiple PCR steps including overlap extensions and insertion of BamHI and SpeI restriction sites flanking the L2 aa 14-122, using primers L2-14-Bam-F (SEQ ID NO. 3: CAGGATCCGCAACC CAACTTTACAAGAC) and L2-122-Spe-R (SEQ ID NO. 2). The HBche-L2 coding sequence was inserted into a geminiviral replicon binary vector pBYR2eK2M (FIG. 3), which includes the following elements: CaMV 35S promoter with duplicated enhancer (Huang et al., 2009), 5′ UTR of N. benthamiana psaK2 gene (Diamos et al., 2016), intron-containing 3′ UTR and terminator of tobacco extensin (Rosenthal et al, 2018), CaMV 35S 3′ terminator (Rosenthal et al, 2018), and Rb7 matrix attachment region (Diamos et al., 2016).

### 2. Recombinant Immune Complex

The recombinant immune complex (RIC) vector was adapted from Kim et al., (2015). The HPV-16 L2 (aa 14-122) segment was inserted into the BamHI and SpeI sites of the gene encoding humanized mAb 6D8 heavy chain, resulting in 6D8 epitope-tagged L2. The heavy chain fusion was inserted into an expression cassette linked to a 6D8 kappa chain expression cassette, all inserted into a geminiviral replicon binary vector (FIG. 3, RIC vector). Both cassettes contain CaMV 35S promoter with duplicated enhancer (Huang et al., 2009), 5′ UTR of N. benthamiana psaK2 gene (Diamos et al., 2016), intron-containing 3′ UTR and terminator of tobacco extensin (Rosenthal et al, 2018), and Rb7 matrix attachment region (Diamos et al., 2016).

ii. Agroinfiltration of Nicotiana benthamiana Leaves

Binary vectors were separately introduced into Agrobacterium tumefaciens EHA105 by electroporation. The resulting strains were verified by restriction digestion or PCR, grown overnight at 30° C., and used to infiltrate leaves of 5- to 6-week-old N. benthamiana maintained at 23-25° C. Briefly, the bacteria were pelleted by centrifugation for 5 minutes at 5,000 g and then resuspended in infiltration buffer (10 mM 2-(N-morpholino)ethanesulfonic acid (MES), pH 5.5 and 10 mM MgSO₄) to OD₆₀₀=0.2, unless otherwise described. The resulting bacterial suspensions were injected by using a syringe without needle into leaves through a small puncture (Huang et al. 2004). Plant tissue was harvested after 5 DPI, or as stated for each experiment. Leaves producing GFP were photographed under UV illumination generated by a B-100AP lamp (UVP, Upland, CA).

iii. Protein Extraction

Total protein extract was obtained by homogenizing agroinfiltrated leaf samples with 1:5 (w:v) ice cold extraction buffer (25 mM sodium phosphate, pH 7.4, 100 mM NaCl, 1 mM EDTA, 0.1% Triton X-100, 10 mg/mL sodium ascorbate, 0.3 mg/mL PMSF) using a Bullet Blender machine (Next Advance, Averill Park, NY) following the manufacturer's instruction. To enhance solubility, homogenized tissue was rotated at room temperature or 4° C. for 30 minutes. The crude plant extract was clarified by centrifugation at 13,000 g for 10 minutes at 4° C. Necrotic leaf tissue has reduced water weight, which can lead to inaccurate measurements based on leaf mass. Therefore, extracts were normalized based on total protein content by Bradford protein assay kit (Bio-Rad) with bovine serum albumin as standard.

iv. SDS-PAGE and Western Blot

Clarified plant protein extract was mixed with sample buffer (50 mM Tris-HCl, pH 6.8, 2% SDS, 10% glycerol, 0.02% bromophenol blue) and separated on 4-15% polyacrylamide gels (Bio-Rad). For reducing conditions, 0.5M DTT was added, and the samples were boiled for 10 minutes prior to loading. Polyacrylamide gels were either transferred to a PVDF membrane or stained with Coomassie stain (Bio-Rad) following the manufacturer's instructions. For L2 detection, the protein transferred membranes were blocked with 5% dry milk in PBST (PBS with 0.05% tween-20) overnight at 4° C. and probed with polyclonal rabbit anti-L2 diluted 1:5000 in 1% PBSTM, followed by goat anti-rabbit horseradish peroxidase conjugate (Sigma). Bound antibody was detected with ECL reagent (Amersham).

v. Immunization of Mice and Sample Collection

All animals were handled in accordance to the Animal Welfare Act and Arizona State University IACUC. Female BALB/C mice, 6-8 weeks old, were immunized subcutaneously with purified plant-expressed L2 (14-122), HBche-L2 VLP, L2 RIC, or PBS mixed 1:1 with Imject® Alum (Thermo Scientific, Rockford, IL). In all treatment groups, the total weight of antigen was set to deliver an equivalent 5 μg of L2. Doses were given on days 0, 21, and 42. Serum collection was done as described (Santi et al. 2008) by submandibular bleed on days 0, 21, 42, and 63.

vi. Antibody Measurements

Mouse antibody titers were measured by ELISA. Bacterially-expressed L2 (amino acids 11-128) was bound to 96-well high-binding polystyrene plates (Corning), and the plates were blocked with 5% nonfat dry milk in PBST. After washing the wells with PBST (PBS with 0.05% Tween 20), the diluted mouse sera were added and incubated. Mouse antibodies were detected by incubation with polyclonal goat anti-mouse IgG-horseradish peroxidase conjugate (Sigma). The plate was developed with TMB substrate (Pierce) and the absorbance was read at 450 nm. Endpoint titers were taken as the reciprocal of the lowest dilution which produced an OD450 reading twice the background. IgG1 and IgG2a antibodies were measured with goat-anti mouse IgG1 or IgG2a horseradish peroxidase conjugate.

vii. Electron Microscopy

Purified samples of HBche or HBche-L2 were initially incubated on 75/300 mesh grids coated with formvar. Following incubation, samples were briefly washed twice with deionized water then negatively stained with 2% aqueous uranyl acetate. Transmission electron microscopy was performed with a Phillips CM-12 microscope, and images were acquired with a Gatan model 791 CCD camera.

viii. Statistical Analysis

The significance of vaccine treatments and virus neutralization was measured by non-parametric Mann-Whitney test using GraphPad prism software. Two stars (**) indicates p values <0.05. Three stars (***) indicates p values <0.001.

b. Design and Expression of HBc VLPs and RIC Displaying HPV16 L2

BeYDV plant expression vectors (FIG. 3) expressing either the target VLP HBche-L2, or L2 and HBche alone as controls, were agroinfiltrated into the leaves of N. benthamiana and analyzed for VLP production. After 4-5 days post infiltration (DPI), leaves displayed only minor signs of tissue necrosis, indicating that the VLP was well-tolerated by the plants (FIG. 4A). Leaf extracts analyzed by reducing SDS-PAGE showed an abundant band near the predicted size of 51 kDa for HBche-L2, just above the large subunit of rubisco (RbcL). HBche was detected around the predicted size of 38 kDa (FIG. 4B). Western blot probed with anti-L2 polyclonal serum detected a band for HBche-L2 at −51 kDa (FIG. 4B). These results indicate that this plant system is capable of producing high levels of L2-containing HBc VLP.

To express L2-containing RIC, amino acids 14-122 of HPV16 L2 were fused with linker to the C-terminus of the 6D8 antibody heavy chain and tagged with the 6D8 epitope (Kim et al. 2015). A BeYDV vector (FIG. 3) expressing both the L2-fused 6D8 heavy chain and the light chain was agroinfiltrated into leaves of N. benthamiana and analyzed for RIC production. To create more homogenous human-type glycosylation, which has been shown to improve antibody Fc receptor binding in vivo, transgenic plants silenced for xylosyltransferase and fucosyltransferase were employed (Castilho and Steinkellner 2012). By western blot, high molecular weight bands >150 kDa suggestive of RIC formation were observed (FIG. 4C). Expression of soluble L2 RIC was lower than HBche-L2 due to relatively poor solubility of the RIC (FIG. 4C).

After rigorous genetic optimization, the N. benthamiana system is capable of producing very high levels of recombinant protein, up to 30-50% of the total soluble plant protein, in 4-5 days (Diamos et al. 2016). Using this system, we produced and purified milligram quantities of fully assembled and potently immunogenic HBc VLPs displaying HPV L2 through a simple one-step purification process (FIGS. 4A-4C and 6).

c. Purification and Characterization of HBche-L2 and L2 RIC

To assess the assembly of HBc-L2 VLP, clarified plant extracts containing either HBche-L2 or HBche were analyzed by sucrose gradient sedimentation. HBche-L2 sedimented largely with HBche, which is known to form VLP, though a small increase in density was observed with HBche-L2, perhaps due to the incorporation of L2 into the virus particle (FIG. 5A). To demonstrate particle formation, sucrose fractions were examined by electron microscopy. Both HBche and HBche-L2 formed ˜30 nm particles, although the appearance of HBche-L2 VLP suggested slightly larger, fuller particles (FIGS. 5C and 5D). As most plant proteins do not sediment with VLP, pooling peak sucrose fractions resulted in >95% pure HBche-L2 (FIG. 5B), yielding sufficient antigen (>3 mg) for vaccination from a single plant leaf.

L2 RIC was purified from plant tissue by protein G affinity chromatography. By SDS-PAGE, an appropriately sized band was visible >150 kDa that was highly pure (FIG. 5B). Western blot confirmed the presence of L2 in this band, indicating proper RIC formation (FIG. 5B). L2 RIC bound to human complement C1q receptor with substantially higher affinity com-pared to free human IgG standard, suggesting proper immune complex formation (FIG. 5E).

d. Mouse Immunization with HBche-L2 and L2 RIC

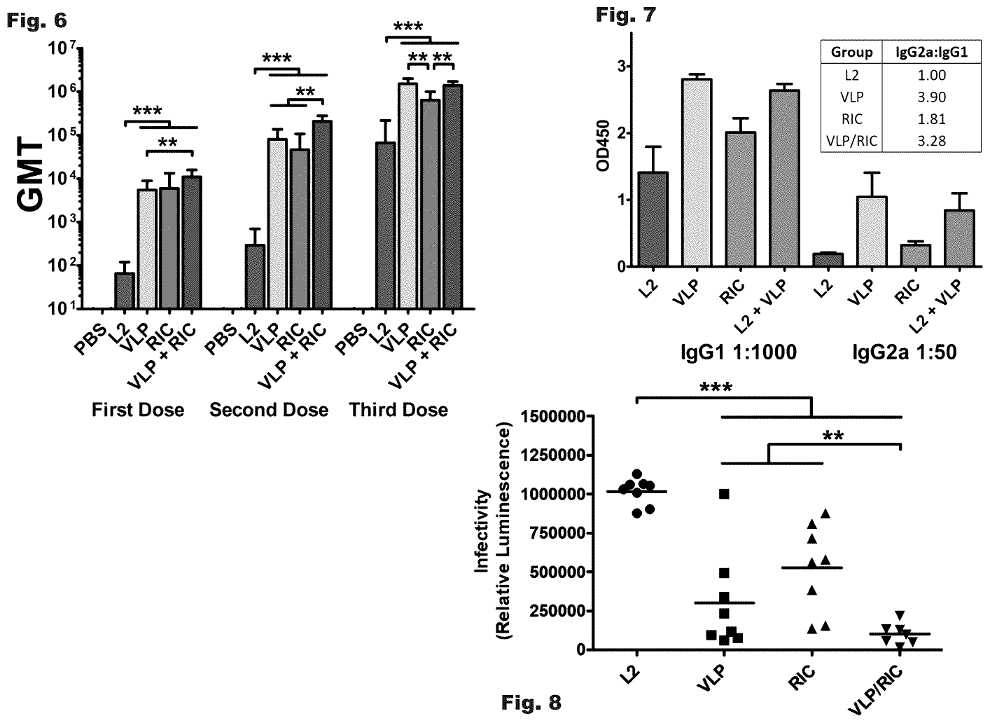

Groups of Balb/c mice (n=8) were immunized, using alum as adjuvant, with three doses each of 5 μg L2 delivered as either L2 alone, HBche-L2 VLP, L2 RIC, or a combination of half VLP and half RIC. VLP and RIC, alone or combined, greatly enhanced antibody titers compared to L2 alone by more than an order of magnitude at all time points tested (FIG. 6). After one or two doses, the combined VLP/RIC treatment group outperformed both the VLP or RIC groups, reaching mean endpoint titers of >200,000, which represent a 700-fold increase over immunization with L2 alone (FIG. 6). After the third dose, both the VLP and combined VLP/RIC groups reached endpoint titers >1,300,000, a 2-fold increase over the RIC alone group. To determine the antibody subtypes produced by each treatment group, sera were assayed for L2-binding IgG1 and IgG2a. All four groups produced predominately IgG1 (FIG. 7, note dilutions). However, RIC and especially VLP-containing groups had an elevated ratio of IgG2a:IgG1 (>3-fold) compared to L2 alone (FIG. 7).

In vitro neutralization of HPV16 pseudovirions showed that the VLP and RIC groups greatly enhanced neutralization compared to L2 alone (FIG. 5, p<0.001). Additionally, VLP and RIC combined further enhanced neutralization activity ($5-fold, p<0.05) compared to either antigen alone, supporting the strong synergistic effect of delivering L2 by both platforms simultaneously.

In this study, by displaying amino acids 11-128 on the surface of plant-produced HBc VLPs, L2 antibody titers as high as those seen with L1 vaccines were generated (FIG. 6). Mice immunized with L2 alone had highly variable antibody titers, with titers spanning two orders of magnitude. By contrast, the other groups had much more homogenous antibody responses, especially the VLP-containing groups, which had no animals below an endpoint titer of 1:1,000,000 (FIG. 6). These results underscore the potential of HBc VLP and RIC to provide consistently potent immune responses against L2. Moreover, significant synergy of VLP and RIC systems was observed when the systems were delivered together, after one or two doses (FIG. 6). Since equivalent amounts of L2 were delivered with each dose, the enhanced antibody titer did not result from higher L2 doses. Rather, these data suggest that higher L2-specific antibody production may be due to augmented stimulation of L2-specific B cells by T-helper cells that were primed by RIC-induced antigen presenting cells. Although treatment with VLP and RIC alone reached similar endpoint titers as the combined VLP/RIC group after 3 doses, virus neutralization was substantially higher (>5-fold) in the combined group (FIG. 8). Together, these data indicate unique synergy exists when VLP and RIC are delivered together. Inventors have observed similarly significant synergistic enhancement of immunogenicity for a variety of other antigens.

Mice immunized with L2 alone had highly variable antibody titers, with titers spanning two orders of magnitude. By contrast, the VLP and VLP/RIC groups had much more homogenous antibody responses, with no animals below an endpoint titer of 1:1,000,000 (FIG. 6). These results underscore the potential of HBc VLP and RIC to provide consistently potent immune responses against L2.

Fc gamma receptors are present on immune cells and strongly impact antibody effector functions such as antibody-dependent cell-mediated cytotoxicity and complement-dependent cytotoxicity (Jefferis 2009). In mice, these interactions are controlled in part by IgG subtypes. IgG1 is associated with a Th2 response and has limited effector functions. By contrast, IgG2a is associated with a Th1 response and more strongly binds complement components (Neuberger and Rajewsky 1981) and Fc receptors (Radaev 2002), enhancing effector functions and opsonophagocytosis by macrophages (Takai et al. 1994). Immunization with L2 alone was found to produce low levels of IgG2a, however immunization with RIC and VLP produced significant increases in IgG2a titers. VLP-containing groups in particular showed a 3-fold increase in the ratio of IgG2a to IgG1 antibodies (FIG. 7). Importantly, production of IgG2a is associated with successful clearance of a plethora of viral pathogens (Coutelier et al. 1988; Gerhard et al. 1997; Wilson et al. 2000; Markine-Goriaynoff and Coutelier 2002).

The glycosylation state of the Fc receptor also plays an important role in antibody function. Advances in glycoengineering have led to the development of transgenic plants with silenced fucosyl- and xylosyl-transferase genes capable of producing recombinant proteins with authentic human N-glycosylation (Strasser et al. 2008). Antibodies produced in this manner have more homogenous glycoforms, resulting in improved interaction with Fc gamma and complement receptors compared to the otherwise identical antibodies produced in mammalian cell culture systems (Zeitlin et al. 2011; Hiatt et al. 2014; Strasser et al. 2014; Marusic et al. 2017). As the known mechanisms by which RIC vaccines increase immunogenicity of an antigen depend in part on Fc and complement receptor binding, HPV L2 RIC were produced in transgenic plants with silenced fucosyl- and xylosyl-transferase. Consistent with these data, we found that L2 RIC strongly enhanced the immunogenicity of L2 (FIG. 6). However, yield suffered from insolubility of the RIC (FIG. 4C). We found that the 11-128 segment of L2 expresses very poorly on its own in plants and may be a contributing factor to poor L2 RIC yield. Importantly, we have produced very high yields of RIC with different antigen fusions. Thus, in some aspects, antibody fusion with a shorter segment of L2 could substantially improve the yield of L2 RIC.

e. Neutralization of HPV Pseudovirions

Neutralization of papilloma pseudoviruses (HPV 16, 18, and 58) with sera from mice immunized IP with HBc-L2 VLP and L2(11-128) showed neutralization of HPV 16 at titers of 400-1600 and 200-800, respectively (Table 1). More mice IP-immunized with HBc-L2 VLP had antisera that cross-neutralized HPV 18 and HPV 58 pseudoviruses, compared with mice immunized with L2(11-128). Anti-HBc-L2 VLP sera neutralized HPV 18 at titers of 400 and HPV 58 at titers ranging from 400-800 (Table 1), while anti-L2(11-128) sera neutralized HPV 18 at a titer of 200 and HPV 58 at a titer of 400 (Table 1). None of the sera from intranasal-immunized mice demonstrated neutralizing activity, consistent with lower anti-L2 titers for intranasal than for intraperitoneal immunized mice.

TABLE 1
L2-specific serum IgG and pseudovirus neutralization
titers from IP immunized mice

| | | Neutralization of Pseudoviruses | | |
|---|---|---|---|---|
| Immunogen | Serum IgG | HPV 16 | HPV 18 | HPV 58 |
| HBc-L2 | >50,000 | 400 | — | — |
| | ~70,000 | 1600 | 400 | 400 |
| | >80,000 | 1600 | 400 | 800 |
| L2 (11-128) | ~8000 | 200 | — | — |
| | ~12,000 | 400 | — | — |
| | ~50,000 | 800 | 200 | 400 |

### Example 2. Influenza Vaccine Compositions

a. Methods
i. Vector Construction

A codon-optimized sequence for the expression of a dimeric human consensus M2e protein (SLLTEVETPIRNEWGCRCNDSSDGGSGGSLLTEVETPIRNEWGCRCNDSSD, SEQ ID NO. 10) in N. benthamiana was designed (Neirynck et al., 1999; Blokhina et al., 2013; Mardanova et al., 2015; Krishnavajhala et al., 2018), and restriction sites for the restriction enzymes BamHI (New England Biolabs) and SpeI (New England Biolabs) and binding sites for the M13F and M13R primers flanking the BamHI and SpeI sites were added to the ends of the sequence to maximize compatibility with existing vectors. M2e monomers were linked by a glycine-serine linker to minimize interference of the linker with the protein and RIC as a whole. Tandem repeats of M2e increases the chance of cross-linking occurring in B cell receptors on B cells and also increases the chances of M2e being degraded and displayed by proteosomal digestion and MHC presentation. This increases the immunogenicity of the vaccine. 250 ng of the sequence was ordered from IDT (Integrated DNA Technologies, Coralville, IA) as a gBlock, which was promptly resuspended by centrifuging the geneblock for 5 seconds at 3,000 xg and resuspending the resulting pellet in TE buffer to a final concentration of 10 ng/μl. The geneblock was then amplified by high-fidelity PCR using M13F and M13R primers and run on a 1% agarose gel, with the resulting band being excised and the DNA contained within being isolated by dissolving the gel fragment in sodium iodide (a chaotropic salt that dissolves agarose) and heating for approximately 10 minutes. The DNA was precipitated out of the solution by mixing the solution with a small amount (7 μl) of silicon dioxide suspension, which binds DNA under high salt conditions. The solution was pelleted, washed with 50% ethanol/50 mM NaCl to remove NaI, and the DNA was eluted from the silicon dioxide with sterile water. Following this, the amplified M2e sequence was then digested with BamHI and SpeL. The vector backbone plasmid, pBYR11eMa-h6D8-L2, was digested with SbfI and SpeL for the vector fragment, and separately with SbfI and BamHI for the 2264 bp fragment. The three fragments were ligated using T4 DNA ligase (New England Biolabs) overnight in a 16° C. water bath.

The ligated plasmid was then precipitated using ammonium acetate and 2-propanol to increase purity and decrease the volume of the plasmid in solution (the precipitation allowed for a volume reduction from 20 μl to 3 μl, vastly increasing the concentration of the plasmid in solution). Following precipitation, 2 μl of the plasmid was electroporated into competent DH5α E. coli, which was allowed to grow in a 2 mL Eppendorf tube containing 500 μl YENB broth for one hour to allow the E. coli to recover. This 500 μl of broth was subsequently plated onto an LB+kanamycin plate and allowed to grow overnight in a 37° C. incubator. Ten colonies were selected and screened via PCR using the 6D8H-F and Ext3-R primers (Table 1) with the two colonies producing the brightest bands on the agarose gel being selected for plasmid preparation via lysis of the cells (using an EDTA-containing buffer to prevent DNAse activity) and precipitation of the plasmid through mixing with ammonium acetate and ethanol to precipitate the DNA. pBYR11eMa-h6D8M2e was isolated via centrifugation and resuspension in TE buffer. Isolated pBYR11eMa-h6D8M2e from the E. coli were Sanger sequenced in the region of the plasmid containing M2e to ensure sequence integrity.

To generate the plasmid encoding the VLP, pBYR2eK2M-HBcheM2e was constructed. pET-28b (Novalgen) and the M2e geneblock were digested with NcoI and XhoI. These fragments were ligated to generate the plasmid pET28b-M2e. Then, the plasmid pBY037P3-HbcheL2ic and the M2e gBlock were digested with BamHI and SpeI, with the fragments being ligated to form pBY037P3-HbcheM2e, which contained M2e fused to the MIR of an HBc monomer. pBY037P3-HbcheM2e was digested with NcoI and SpeI to obtain the 926 bp fragment. pBYR2eK2M-HbcheZE3 was digested with SbfI and SpeI for the vector fragment, and separately with SbfI and NcoI to obtain the 821 bp fragment with promoter and 5′UTR. These fragments were ligated together to form pBYR2eK2M-HbcheM2e, which contained the HBc dimer with M2e inserted into the second HBc monomer's MIR region.

Table 2 lists nucleotide sequences used in the construction of the vectors.

TABLE 2
Summary of nucleic acid sequence used.

| Name | Sequence from 5′ to 3′ |
|---|---|
| M2e gBlock, | GTAAAACGACGGCCAGTGGATCCTCTTTGC |
| SEQ ID NO. 11 | TTACCGAGGTTGAGACCCCTATTAGAAACG |
| | AGTGGGGTTGCAGATGTAACGATTCTTCCG |
| | ACGGaGGtTCTGGaggtTCCCTTTTGACTG |
| | AAGTgGAGACTCCAATcAGgAACGAATGGG |
| | GATGcAGATGCAACGACTCCTCTGACGGAG |
| | GTGGAactagtCATGGTCATAGCTGTTTCC |
| M2e-Nco-F Primer, | tagccatgGGATCCTCTTTGCTTACCG |
| SEQ ID NO. 12 | |
| M2e-Xho-R Primer, | tcgctcgagactagtTCCACCTCCGTC |
| SEQ ID NO. 13 | |
| 6D8H-F Primer, | TGAGGCTCTTCACAATCA |
| SEQ ID NO. 14 | |
| Ext3-R Primer, | CTTCTTCTTCTTCTTTTCTCATTGTC |
| SEQ ID NO. 15 | |
| Ext3i-R Primer, | CAATTTGCTTTGCATTCTTGAC |
| SEQ ID NO. 16 | |
| M13-F Primer, | GTAAAACGACGGCCAGT |
| SEQ ID NO. 17 | |
| M13-R Primer. | GGAAACAGCTATGACCATG |
| SEQ ID NO. 18 | |

ii. Agroinfiltration

After verifying the presence of M2e in pBYR11eMa-h6D8M2e and pBYR2eK2M-HBcheM2e, the plasmids were electroporated into Agrobacterium tumefaciens EHA 105 cells, which were allowed to recover in 500 μl YENB broth for one hour. The cells were then plated on LB+kan plates and incubated at 28° C. for two days. Following this, cultures of the transformed A. tumefaciens were grown overnight at 28° C. on a shaker in YENB, rifampicin (2.775 μg/ml) and kanamycin (50 μg/ml). These cultures were PCR screened after which cultures identified to contain pBYR11 eMa-h6D8M2e and pBYR2eK2M-HBcheM2e were spun down and resuspended in 1× infiltration buffer to an OD of 0.260. Three GnGn N. benthamiana plants (Strasser et al., 2008) ranging from five to six weeks old were infiltrated (specifically in the leaves) with the A. tumefaciens suspensions (Huang and Mason, 2004) and allowed to grow at room temperature for five days. The plants were watered daily.

iii. Extraction and Purification of Recombinant Influenza A Vaccines

Five days after agroinfiltration, plant leaves were homogenized with an electric blender in ice cold buffer (100 mM tris, 50 mM NaCl, 10 mM EDTA, 2 mM PMSF, 0.1 Triton, pH=8.0). No significant necrosis was observed in any of the infiltrated or uninfiltrated leaves. The blended plant leaves were then stirred for 20 minutes at 4° C., after which the solution was filtered through four-ply miracloth to remove plant fibers. 1 ml of this solution was taken as a sample of ‘crude extract’ and frozen at −80° C. for later analysis. 1M phosphoric acid was then added to lower the pH of the extraction to 4.6 for one minute to precipitate plant proteins like RuBisCo, with 2M Tris Base being added to raise the pH of the sample back up to 7.6. The extraction was then centrifuged at 16,000×g for 20 minutes at 4° C. The supernatant was isolated and centrifuged for another 30 minutes at 16,000×g at 4° C. Then, the supernatant was again isolated and centrifuged for a final 10 minutes at 16,000×g at 4° C. to remove as much precipitated plant protein and other insoluble matter in the extraction as possible (as RICs are present in the soluble fraction). The supernatant was then run through vacuum filter sterilizers (pore size=0.45 micron) to remove any remnant bacteria that might have remained after centrifugation.

For the M2e-RIC, a protein G resin column (containing protein G conjugated to agarose beads) (Thermo Fisher Scientific, Waltham, MA, USA) was prepared by running the RIC extraction buffer through the column. The extraction was then run through the prepared column, after which the column was washed again with the RIC extraction buffer again to remove any final contaminants from the column. The RICs were then eluted from the column using a glycine solution (100 mM glycine, pH=2.5), with five 1.5 ml elutions being taken from the column and pH-neutralized using Tris base (100 μl of 1M Tris pH=8.0 in each elution). 50 μl aliquots from each elution were immediately frozen in a −80° C. freezer for later analysis to prevent the larger elutions from degrading due to frequent freezing and thawing. Additionally, small volumes from each elution were used in spectrophotometry to determine the concentration of RICs present in each elution.

For the M2e-VLP, a sucrose gradient and subsequent ultracentrifugation was used to purify the VLPs out of the plant extract as described herein elsewhere. Briefly, 6 mL of extract was centrifuged at 148,000×g for 2.5 hours at 4° C. through a 13 ml sucrose cushion gradient composed of layered 25% and 70% sucrose in phosphate-buffered saline (PBS). VLPs, which have a density between the density 25% and 70% sucrose, was extracted out of the cushion and dialyzed against PBS for further purification and to remove residual sucrose. The use of a sucrose cushion allows for more gentle purification of the VLPs and can increase the yield of the purification (Peyret et al., 2015). The purified M2e-VLPs were then analyzed via spectrophotometry and frozen at −80° C.

An Eppendorf BioPhotometer™ RS232c was used to determine the concentration of RICs within each elution. Using the Beer-Lambert Law (A=F*b*c, where A=absorbance, F=extinction coefficient, b=length of the path of the light in centimeters, and c=concentration), concentration can of the RIC can be determined by rearranging the equation to solve for ‘c’. The extinction coefficient of human IgG is approximately 1.4 (Eisenberg, 1976), and with the length of the path of the light being 1 cm, A₂₈₀ values can be used to determine concentration.

iv. Western Blotting and Coomassie Staining

For the RIC, samples of crude extract, the extract post-filtration, the extract post-acid precipitation, the wash buffer, and five elutions, and a protein ladder standard (GOLDBIO BLUEstain) were run on two 10% SDS-PAGE gels (Bio-Rad) simultaneously under non-reducing conditions. Fresh SDS was added to the running buffer to maximize resolution. One gel was stained using Coomassie Brilliant Blue dye for an hour, after which the gel was destained overnight using deionized water.

The other gel was used to transfer proteins to a PVDF membrane for 20 minutes at 110V. The membrane was blocked in 5% PBSTM (1× phosphate-buffered saline (PBS) containing tween and 5% skim milk) overnight at 4° C., after which the membrane was rinsed in deionized water three times. Then, the membrane was rotated in a 37° C. incubator in a 1% PBSTM (1×PBS and tween and 1% skim milk) solution containing mouse anti-6D8 antibody (Wilson et al., 2000) at a 1:2000 dilution to detect the 6D8 epitope tag on the RIC (Phoolcharoen et al., 2011). Following this, the membrane was washed again in deionized water and incubated and rotated at 37° C. in a 1% PBSTM solution containing goat anti-mouse antibody conjugated to horseradish peroxidase (Sigma) at a 1:500 dilution for one hour. After this, the membrane was washed in deionized water and exposed to a mixture of developing reagents. The membrane was used to develop photosensitive film at an exposure time of 1 minute in the dark.

Additional SDS-PAGE gels were run under similar conditions and using an anti-M2e antibody, MAb 65 (Kolpe et al., 2018) as the primary antibody for western blotting and mouse-anti-kappa chain antibody as the secondary antibody. MAb 65 was expressed in plants and purified in-house. Both RICs and VLPs were probed for the presence of M2e.

v. Electron Microscopy

Purified samples of the M2e VLP were incubated on 75/300 mesh grids coated with formvar and washed twice with deionized water. The VLPs were then negatively stained with 2% aqueous uranyl acetate and analyzed using transmission electron microscopy (TEM). TEM was performed with a Phillips CM-12 microscope, and images were acquired with a Gatan model 791 CCD camera.

vi. Immunization of Mice

All mice were handled in compliance with ASU IACUC regulations and in accordance with the Animal Welfare Act. Groups of 6 female Balb/c mice, 6-8 weeks old, were immunized subcutaneously with three doses of antigen, each containing an equal mass of 5 μg of M2e presented on either VLPs or a 1:1 ratio of the M2e-RIC and M2e-VLP. Doses were administered in a 1:1 ratio with the alum adjuvant Imject Alum (Thermo Fisher Scientific, Waltham, MA). Doses were administered on day 0, 28, and 56, and serum collection was done as described (Santi et al., 2008) by submandibular bleed on days 0, 28, 56, and 86.

vii. Antibody Quantification

Mouse sera were analyzed via enzyme-linked immunosorbent assay (ELISA). 100 μl of a stock solution of 1 mg/ml synthetic monomeric human consensus M2e peptide (GenScript Biotech Corp., NJ) was diluted into 5.8 ml of 50 mM carbonate-bicarbonate buffer (15 mM Na₂CO₃, 35 mM NaHCO₃, pH=9.6) (Ebrahimi et al., 2012) to generate a 17 μg/ml solution of M2e peptide in carbonate buffer. 50 μl of this solution was used to coat each well of 96-well plates (850 ng/well) overnight at 4° C. Following this, plates were allowed to warm to room temperature for 20 minutes, after which the plates were rinsed thrice with PBST and blocked with 100 μl of 5% PBSTM per well at room temperature for 15 minutes. Mouse sera were diluted in 1% PBSTM to dilutions ranging from 1:50 to 1:6250 for dose one, 1:8,000 to 1:1,000,000 for dose two, and 1:40,000 to 1:5,000,000 for dose three. After blocking with 5% PBSTM, 50 μl of diluted mouse sera were added to each well and the plate was incubated overnight at 4° C. The following day, the plates were incubated at 37° C. for 20 minutes and then rinsed thrice with PBST. Following this, a mixture of goat anti-mouse IgG2a antibodies, goat anti-mouse IgG1 antibodies, goat anti-mouse kappa chain antibodies, and goat anti-mouse IgG antibodies, all conjugated to HRP (Santa Cruz Biotechnology Inc., TX) in 1% PBSTM was prepared. Each antibody was present in the solution at a 1:5700 dilution. This solution was used to detect the total antibody titers within each sera sample by adding 50 μl of the mixture to each well and subsequently incubating the plates at 37° C. for 1 hour. Furthermore, additional plates used to determine the titers of IgG2a and IgG1 within each sample. Plates were rinsed five times with PBST and incubated for 45 minutes with 50 μl of TMB (3,3′,5,5′-Tetramethylbenzidine) being added to each well. After this, the TMB-HRP reaction was stopped through the addition of HCl and the absorbance of the plates were read using a Molecular Devices SpectraMax 340PC Microplate Reader at 450 nm. Endpoint titers were calculated using GraphPad Prism (GraphPad Sofware, Inc.) to calculate the geometric mean of the ELISA results to determine geometric mean titers.

viii. Analysis of Cytokine Production in Mice

Mouse splenocytes were removed and homogenized via mashing in a 70 μm nylon strainer with the plunger of a 3 ml syringe. The strainer and plunger were then washed using 13 ml of RPMI complete (RPMI, 10% heat-inactivated PBS, 1% P/S/G) into a microcentrifuge tube to collect cells. Cells were then centrifuged for 5 mins at 1200 RPM at 4° C., with the supernatant being removed thereafter and the cells being resuspended in 2 ml red cell lysis buffer (ACK) and incubated for 2 minutes at room temperature. The cells were then quenched in 8 ml RPMI complete and centrifuged again for 5 minutes at 1200 RPM at 4° C. The cell pellet was then washed twice with 10 ml RPMI complete and subsequently resuspended in 2 ml RPMI.

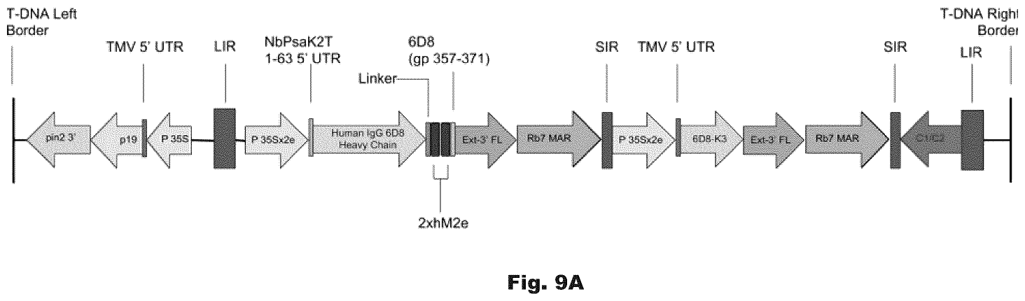

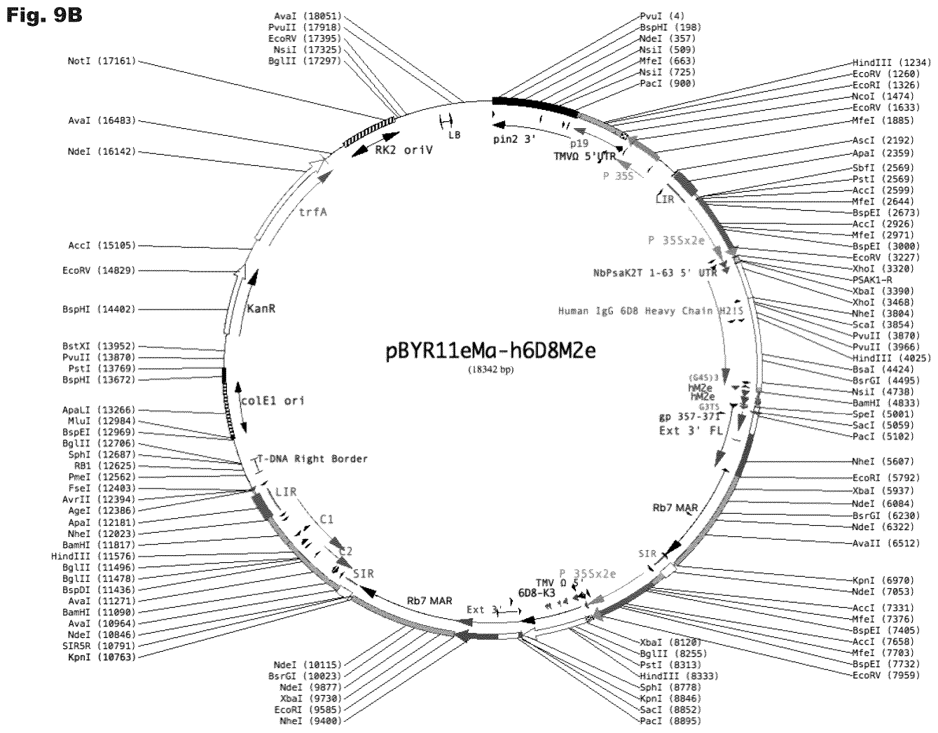

Splenocytes were then plated in 96-well round bottom plates at a concentration of 106 cells per well. The plate was then centrifuged and the splenocytes were resuspended in 180 μl of assay media (RPMI complete, 1.11 ng/ml human IL-2, 5.5 μl/ml GolgiPlug (BD Biosciences-US)). Splenocytes were then exposed to either 20 μl of 10 μg/ml synthetic M2e peptide (GenScript Biotech Corp., NJ), 20 μl of RPMI complete, or 20 μl PMI/ionomycin) and incubated at 37° C. for 5 hours. Following incubation, cells were pelleted at 1300 rpm for 3 minutes, the supernatant was removed, and cells were washed with 1× fluorescence-activated cell sorting (FACS) buffer. Cells were stained with anti-CD8 (1:100) and anti-CD4 (1:100) in 100 μl FACS buffer and incubated for 30 minutes at 4° C., after which cells were washed twice with FACS buffer to remove excess unbound stain. The cells were then fixed and permeabilized through resuspension in 100 μl of Fixation/Permeabilization solution (BD Biosciences, USA). Cells were then stained for intracellular cytokines using 50 μl of staining solution; CD4 responses were assayed via staining in a solution containing 1:100 dilutions of anti-IL-4, anti-IL-21, and anti-IFN-γ in Permeabilization/Wash buffer (BD Biosciences, USA). while CD8 responses were assayed via staining with a solution containing 1:100 dilutions of anti-IL-2, anti-TNFα, and anti-IFN-γ in Permeabilization/Wash buffer. Cells were then washed twice with Permeabilization/Wash buffer, resuspended in 200 μl FACS buffer, and analyzed via FACS using an LSR Fortessa (FIG. 9).

b. Construction of Recombinant Influenza Vaccines

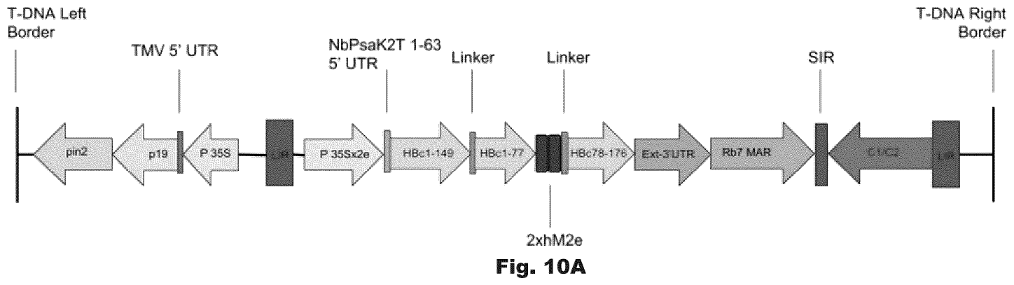

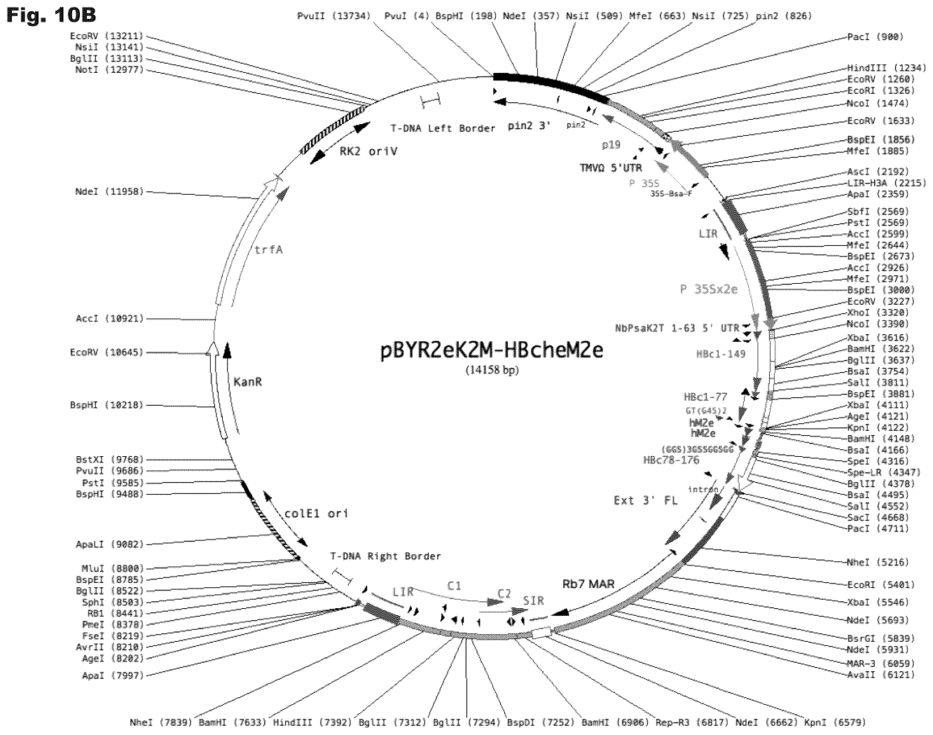

Two recombinant universal influenza A vaccines were developed and expressed using Agrobacterium tumefaciens-mediated transfer of geminiviral vectors into glycoengineered Nicotiana benthamiana plants (Strasser et al., 2008). The vaccines, using both the recombinant immune complex (RIC) and hepatitis B core antigen (HbcAg) virus-like particles (VLP) as vaccine platforms to boost immunogenicity, presented a consensus sequence of the ectodomain of the matrix 2 protein of human influenza A, M2e (SLLTEVETPIRNEWGCRCNDSSD, SEQ ID NO. 9). The antigen was constructed as a dimer, with a 2×GGS linker linking the two copies of M2e together to minimize steric hindrance and other unwanted interactions between the two copies, codon-optimized for expression in Nicotiana benthamiana, and inserted into the C-terminal end of the human IgG 6D8 heavy chain gene encoded in pBYR11eMa-h6D8M2e (FIGS. 9A-9B) and into the MIR of the C-terminal copy of the tandem dimer HBcAg encoded in pBYR2eK2M-HBcheM2e (FIGS. 10A-10B), both geminiviral vector plasmids containing several elements to enhance transcription and protein expression.

c. Production of the M2e-RIC and M2e-VLP in Plants

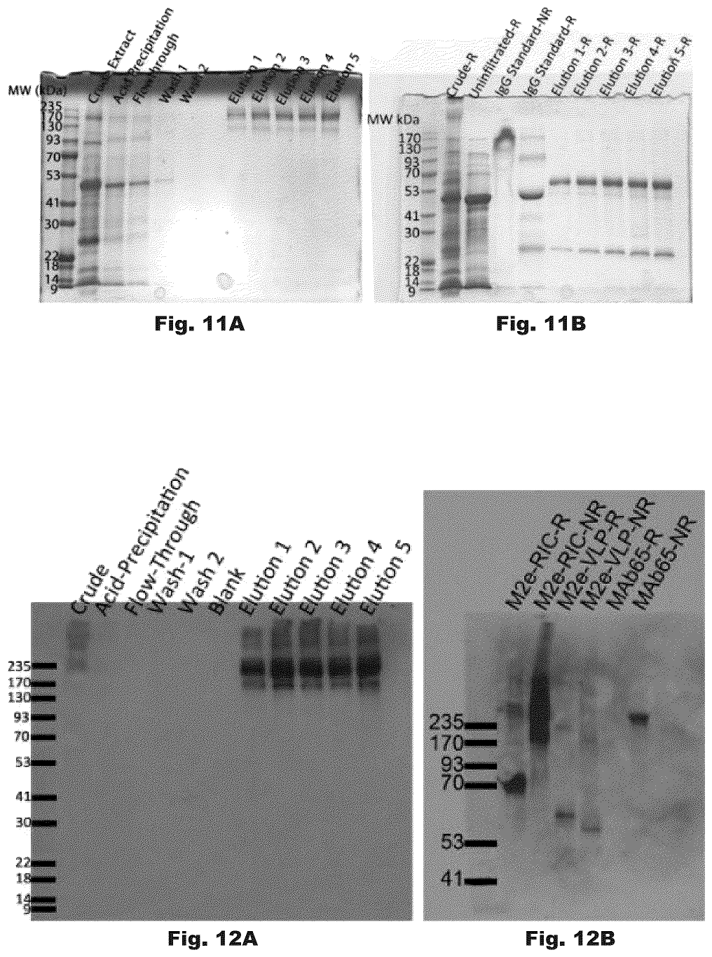

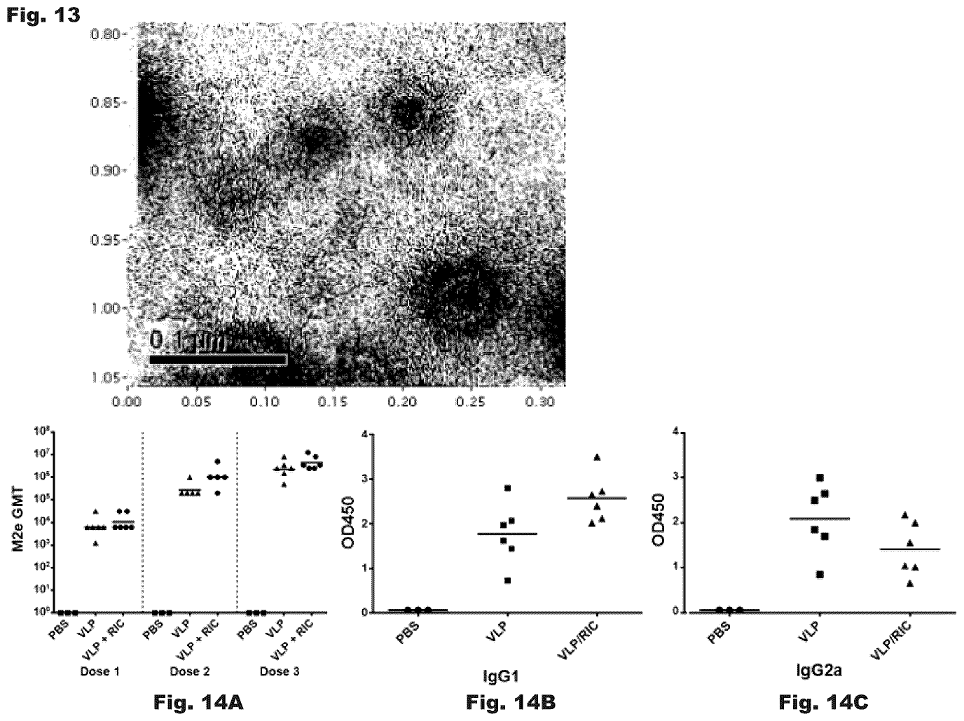

After the expression and purification of the recombinant vaccines via protein G chromatography for the RIC and sucrose gradient purification and dialysis for the VLP, samples were characterized via SDS-PAGE and subsequent Coomassie Brilliant Blue staining and western blotting (FIGS. 11 and 12). The RIC was probed using anti-6D8 antibody and anti-M2e antibody while the VLP was probed solely with anti-M2e antibody. Samples were compared to a standard protein ladder, with the RIC being further compared to an IgG standard to elucidate the suspected differences between the heavy chains and the light chains of the RIC and the standard. Samples probed with the anti-M2e probe demonstrated a clear signal, indicating that both the RIC and the VLP contained the M2e antigen. Further, RICs probed with the anti-6D8 epitope tag demonstrated the presence of the epitope tag. Signal above the expected 164 kDa could be interpreted as suggesting the presence of complex formation, though additional studies to characterize the structure of the RIC binding to other RICs would be necessary to determine whether these bands are indicative of complex formation of aggregation driven by other, unexpected factors. Regardless, the results of these characterization studies confirmed the presence of the target antigen, M2e, and other characteristics of the vaccines. Furthermore, M2e-VLPs were analyzed using TEM, with the images generated confirming the structure of VLP (FIG. 13).

d. Analysis of Mouse Sera and Splenocytes

Two groups of five BALB/c mice were immunized with either the M2e-VLP alone or a combination of the M2e-RIC and the M2e-VLP at days 0, 28, and 56, with bleeds at 0, 28, 56, and 86. Mouse sera was analyzed via ELISA, with total antibody titers being measured after each bleed, and IgG1, and IgG2a titers being measured at the conclusion of day 63. Total antibody titers were consistently 2-3 times higher at all time points in the M2e-RIC/M2e-VLP combination group, though the ratio of IgG2a to IgG1, was lower in the combination group relative to the group that received the M2e-VLP alone (FIGS. 14A-14C).

Both IgG1 and IgG2a play important roles in viral immunity against influenza; in one study, mice with high expression of IgG1 had lower lung viral titers and high influenza virus neutralization, but lower survival rate when challenged with significantly high doses of influenza virus (Huber et al., 2006). Mice that had both isotypes fared the best, though it was noted that mice that had low IgG1 expression and high IgG2a had the same survival rates as those that had equivalent expression of both IgG1 and IgG2a (Huber et al., 2006). This could be due to IgG2a antibodies' propensity to stimulate complement activation much more effectively than IgG1 in mice (Neuberger & Rajewsky, 1981). The data presented suggest that, if mice were challenged with high doses of influenza, those that received the VLP only would have better outcomes than those receiving both groups.

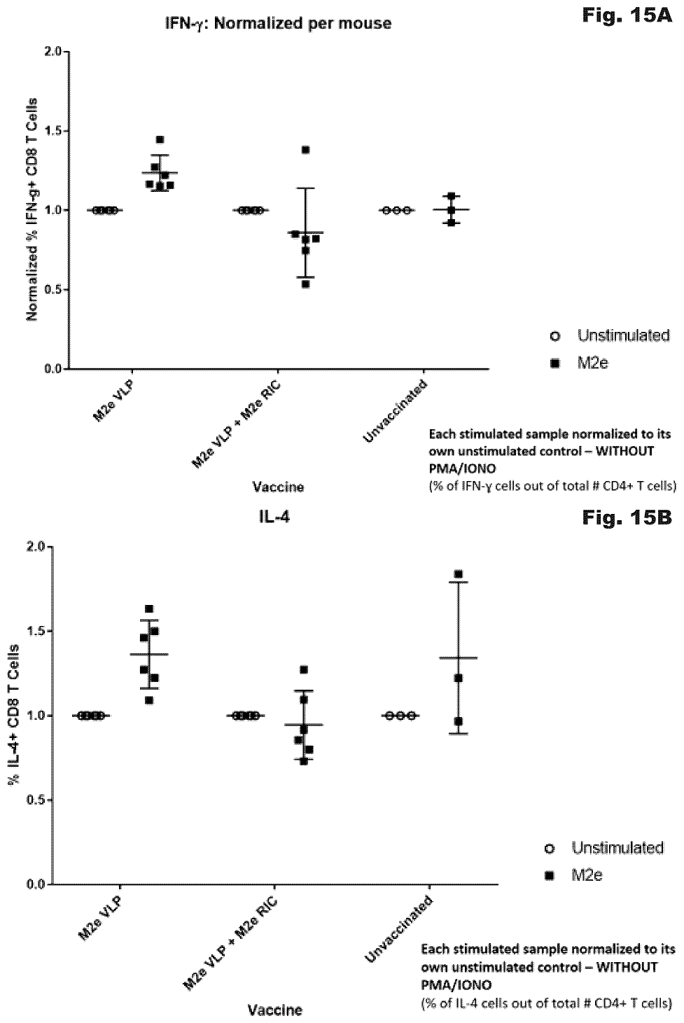

Splenocyte analysis revealed that mice vaccinated with the M2e-VLP had higher levels of IFN-γ and IL-4 positive splenocytes when stimulated with synthetic M2e peptide than mice vaccinated with both vaccines. Interestingly, mice receiving the combination vaccine had, on average, lower proportions of IFN-γ and IL-4 positive splenocytes post-stimulation than both pre-stimulation cells and cells from unvaccinated mice (FIGS. 15A-15B). Th1 cytokines, like IFN-7, induce isotype switching to IgG2a, and Th2 cytokines, like IL-4, a cytokine associated with deleterious effects on viral clearance due to its mediation of down-regulation of antiviral cytokine expression (Sharma et al., 1996), induce isotype switching to IgG1 (Mossman & Coffman, 1989). Thus, the fact that IL-4 production increased to a level higher than that of IFN-γ after stimulation with M2e peptide suggests that the mice receiving the VLP alone may have been undergoing an isotype switch to a Th2-biased phenotype.

### Example 3. N-Terminal Recombinant Immune Complex

Although conventional RICs consists of an antibody, linked via its C-terminus, to an antigen that is followed by an epitope tag for the antibody, the versatility of the RIC platform can be expanded by fusing antigens to the N-terminus of the antibody in an RIC. Thus, antigens with inaccessible N-termini can now be easily used in RICs.

a. Vector Creation and Expression of N-RIC

A bean yellow dwarf expression vector containing dual-expression cassettes was used to create the N-RIC (Kim et al., 2015).

One cassette contained the antigenic coding sequence (either Zika soluble envelope protein (ZsE) or domain zika virus domain III protein (ZDIII)) fused via a short linker to the standard RIC antibody heavy chain that was in turn linked to the antibody epitope tag. The second cassette contained the antibody light chain. Following confirmation of the recombinant plasmid by PCR and restriction digests, the plasmid was transformed into Agrobacterium tumefaciens strain EHA105 and confirmed by PCR.

Agro cultures were grown overnight in YENB+appropriate antibiotics for selection and used for infiltration of 4- to 6-week-old N. benthamiana plants.

b. Confirmation of N-RIC Assembly/Purification

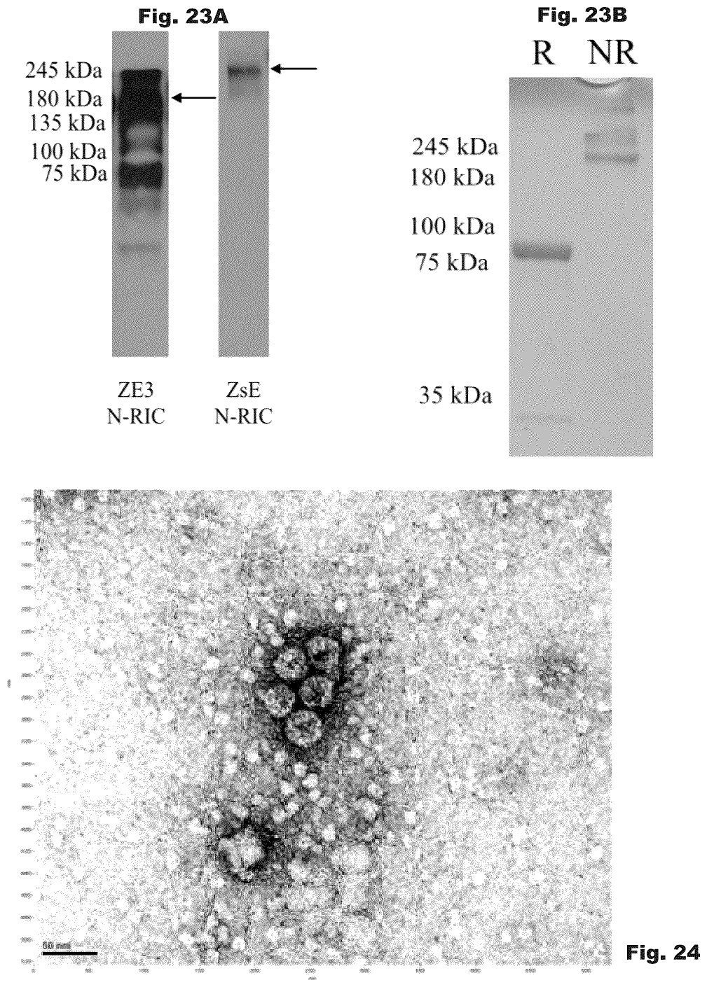

The leaves of transformed N. benthamiana were harvested 4-5 days post infiltration, and the extracted protein were used for a Western blot. The Western blot results showed appropriate assembly of both the ZsE N-RIC and ZDIII N-RIC (FIG. 23A). The ZDIII construct had higher yield, so it was chosen for further study. Following a large-scale infiltration of the ZDIII N-RIC construct, Protein G affinity chromatography was used to purify the construct. The 75 grams of leaf material used for the purification yielded over 4 mg of highly purified material.

Accordingly, an antigen (of various sizes) can be fused to the N-terminus of the RIC antibody.

c. Mice Immunization Trials

Immunization with ZDIII as both the N-RIC and C-RIC had highly comparable immune responses in mice as measured by total antibody titers and a plaque reduction neutralization test with live Zika virus. To test whether the ZDIII N-RIC produces a comparable immune response as a standard RIC (C-RIC), a mouse immunization trial was conducted with both the ZDIII N-RIC construct and a previously created ZDIII C-RIC. Six Balb/C mice were given three doses of either ZDIII N-RIC or RIC over an 8-week period. Serum samples were collected and the antibody titers determined by ELISA. The terminal bleed serum samples were collected a little over a month after the third dose. The ELISA results showed that both the N-RIC and C-RIC produced comparable antibody titers. A plaque reduction neutralization test conducted with live ZIKV virus showed that similar neutralization activity was seen following immunization with either RIC configuration.

### Example 4. Zika Virus Vaccine Compositions

a. Methods
i. Vector Construction

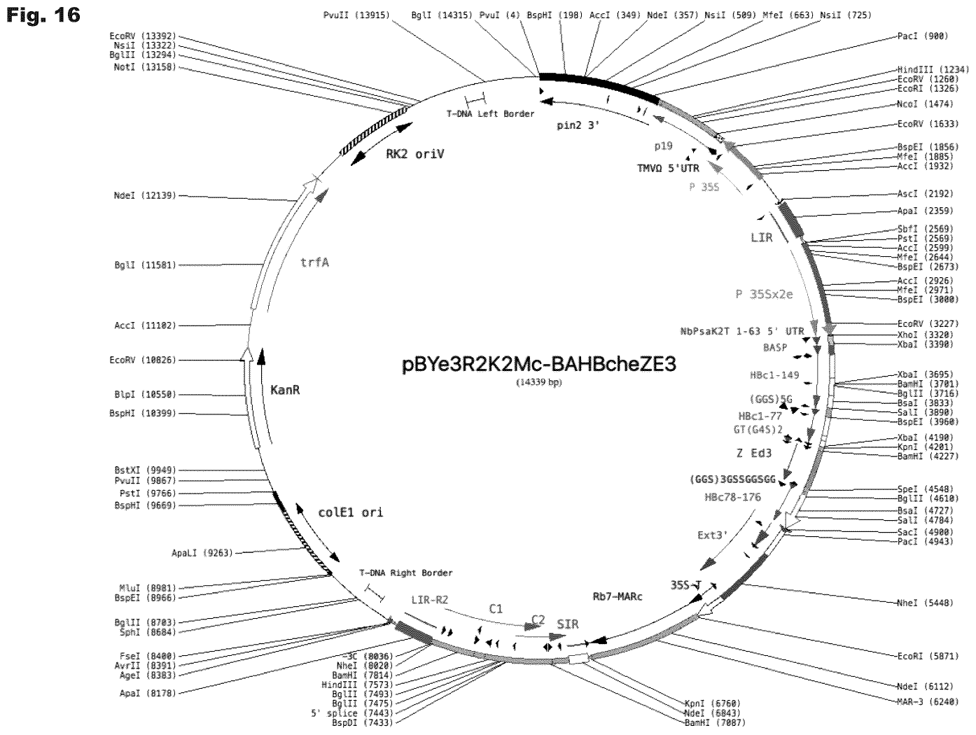

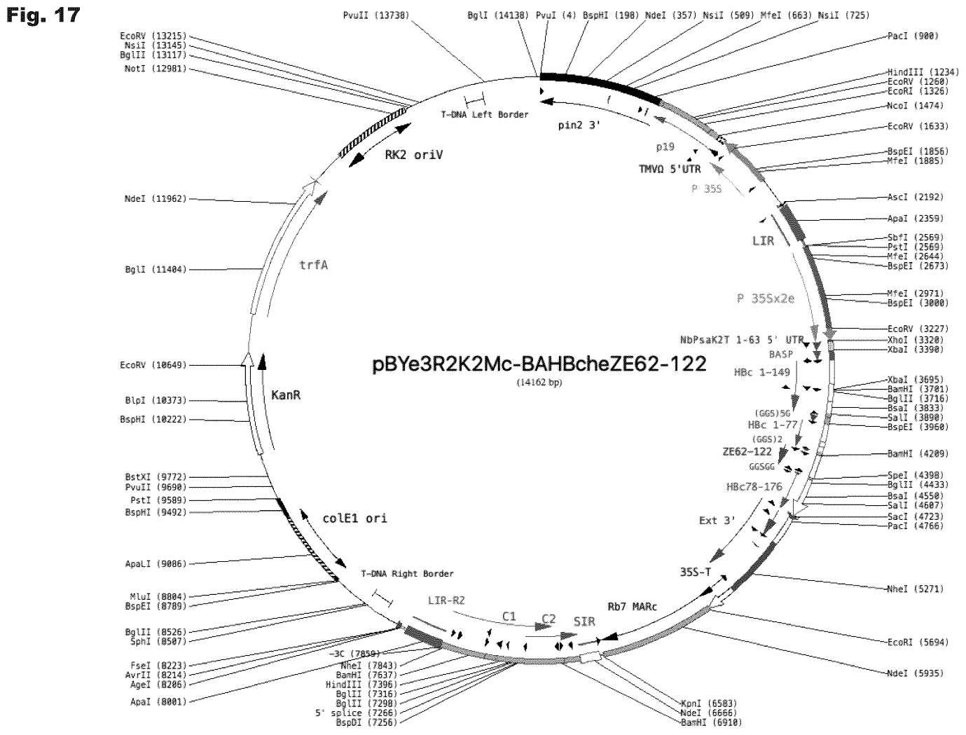

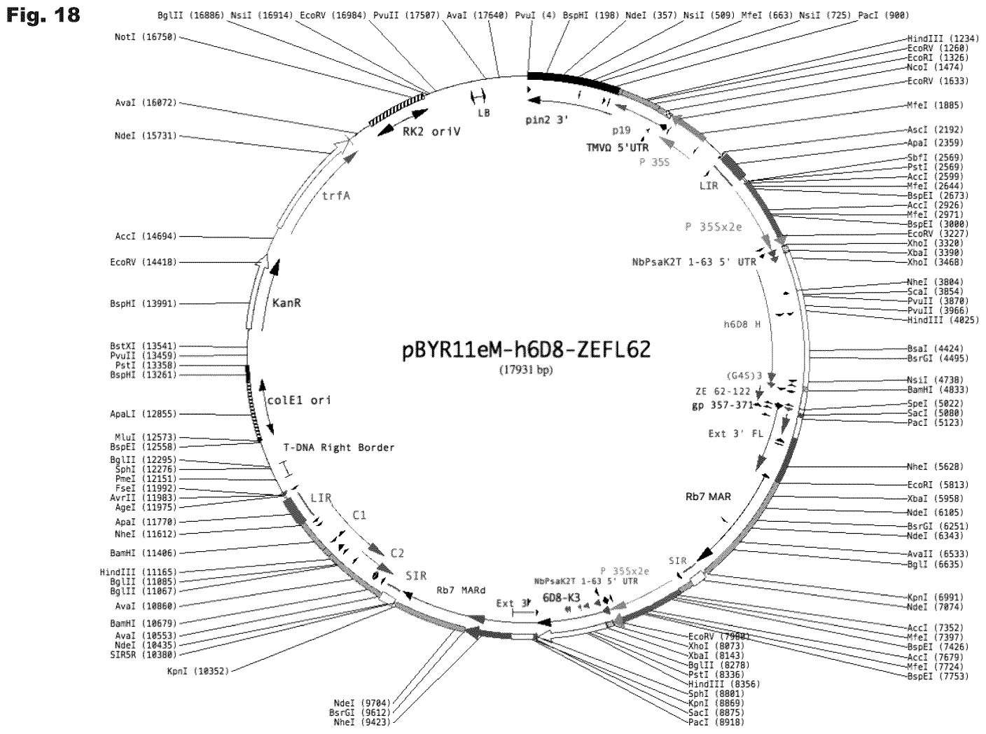

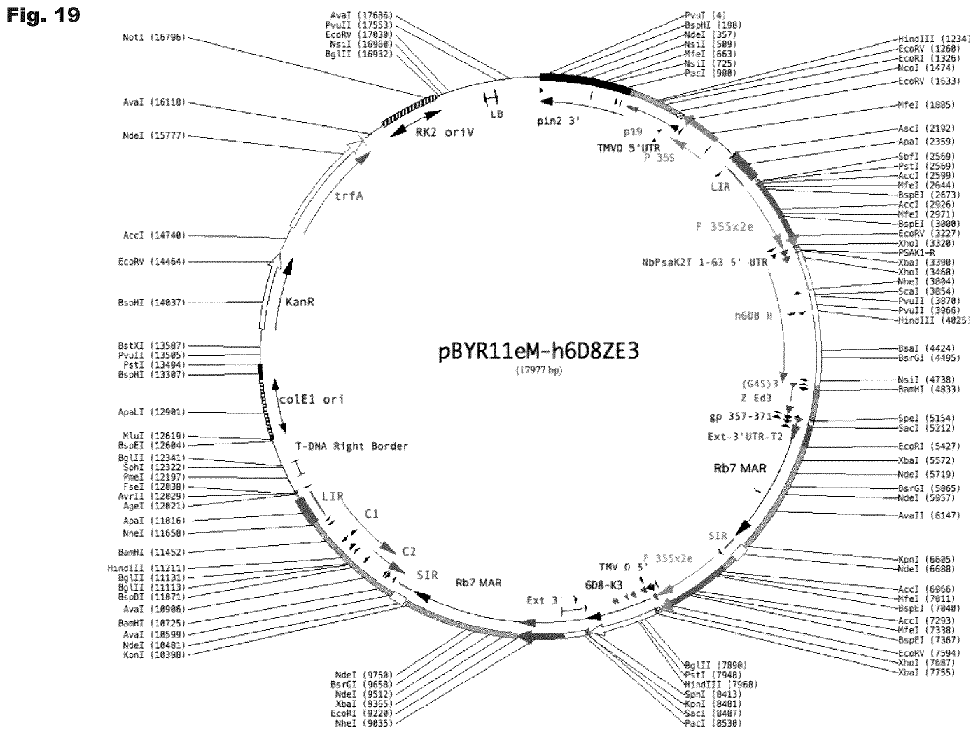

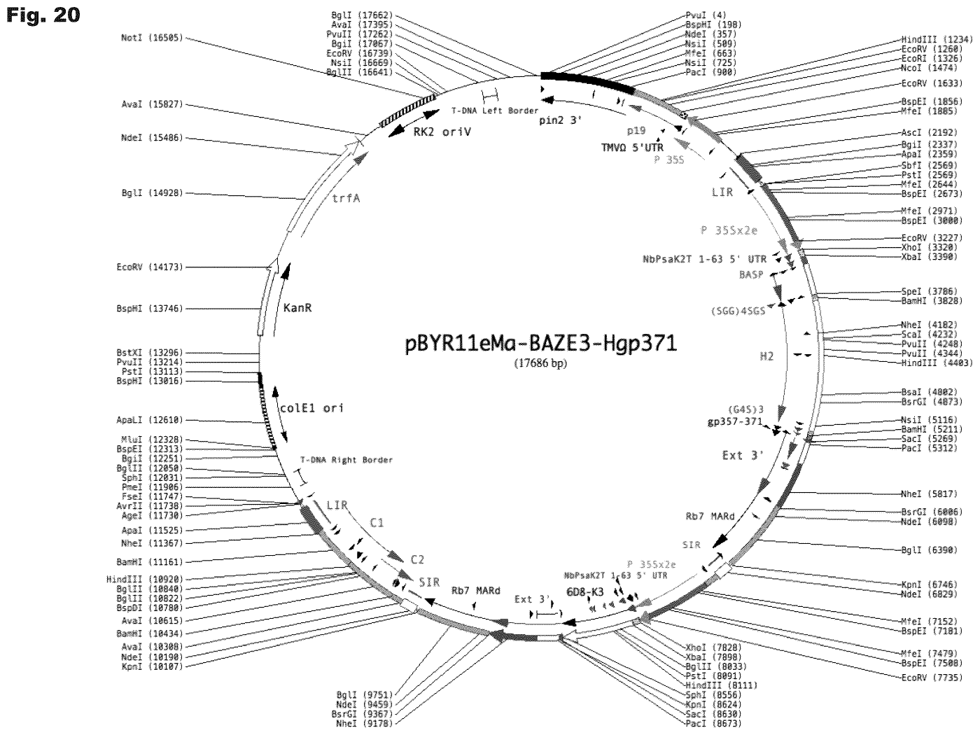

The maps of all expression vectors are provided in FIGS. 16-20, and the sequences are provided in SEQ ID Nos. 34-38. Table 3 lists the oligonucleotide used.

TABLE 3

| | | SEQ |
|---|---|---|
| Oligo name | Sequence (5′ to 3′) | ID NO. |
| 35S-F | AATCCCACTATCCTTCGC | 21 |
| BASP-G-Bsa-R | GCGGTCTCCACCAGAAGCA | 22 |
| | AGAGAAGC | |
| Ext3-R | CTTCTTCTTCTTCTTTTCT | 15 |
| | CATTGTC | |
| H2-Bam-F | GTCGGATCCGATGTTCAGC | 23 |
| | TTCTTGAGTCTGGAG | |
| HBc176-Sac-R | GCGAGCTCTTATCTACGCC | 24 |
| | TAGGAGATGGGGA | |
| HBc-Bsa-F | GCGGTCTCGTGGTATGGAC | 25 |
| | ATTGACCCTTACA | |
| LIR-H3A | AAGCTTGTTGTTGTGACTC | 26 |
| | CGAG | |
| Spe-L-Bam-F | CTAGTGGTGGATCAGGAGG | 27 |
| | TTCTGGTGGTTCTGGAGGT | |
| | TCAG | |
| Spe-L-Bam-R | GATCCTGAACCTCCAGAAC | 28 |
| | CACCAGAACCTCCTGATCC | |
| | ACCA | |
| ZE3-Bam-F | GCGGGATCCAAGGGCGTGT | 29 |
| | CATACTCC | |
| ZE3-Bsa-F | GGGGTCTCGTGGTAAGGGC | 30 |
| | GTGTCATACTC | |
| ZE3-Spe-R | CCGACTAGTGCTACCACTC | 31 |
| | CTGTG | |
| ZEE62-Bam-F | GAGGGATCCGAGGCTTCAA | 32 |
| | TTTCAGACATG | |
| ZES122-Spe-R | GGGACTAGTGGAGCAAGCG | 33 |
| | AATTTAGC | |

1. pBYRIleM-h6D8ZE3 (ZE3 C-Terminal RIC)

A recombinant immune complex consisting of the humanized 6D8 mAb (h6D8) linked via the heavy chain C-terminus to the ZE3 antigen which is in turn linked to the 6D8 epitope tag. This construct is very similar to pBYR11eMa-h6D8-L2.

The coding DNA sequence of ZIKV E protein (GenBank Accession No. AMC13911) was synthesized with optimized N. benthamiana codons (Integrated DNA Technologies, Iowa, USA). DNA encoding E protein domain III (ZE3, amino acids K591-T696) was PCR end-tailored to contain a BamHI site at 5′ and SpeI site at 3′ with primers ZE3-Bam-F and ZE3-Spe-R (Table 3), digested with BamHI and SpeI, and ligated with pBYR11eMa-h6D8-L2 digested BamHI/SpeI to yield pBYR11 eM-h6D8ZE3.

2. pBYR11eMa-BAZE3-Hgp371 (ZE3 N-Terminal RIC)

A recombinant immune complex consisting of the humanized h6D8 Ebola mAb fused via the heavy chain N-terminus to the C-terminus of the ZE3 antigen. The C-terminus of the antibody is linked to the 6D8 epitope tag.

The construct was assembled by ligation of six DNA fragments:
pBYR11 eMa-h6D8-L2 was digested SbfI-XhoI for the vector fragment.
A DNA sequence encoding the promoter, 5′UTR and barley alpha amylase signal peptide (BASP) was amplified from template pBYR2eK2M-6HplcCnetB (Hunter et al., 2019) with primers LIR-H3A and BASP-G-Bsa-R and then digested with SbfJ and BsaI.
The ZE3 coding sequence was PCR end-tailored using primers ZE3-Bsa-F and ZE3-Spe-R, and the product was digested with BsaI and SpeI.
pLIT-L-BamH2 was digested SpeI and BsaI to obtain the 1016 bp fragment containing a linker and 5′ end of the heavy chain sequence. pLIT-L-BamH2 was made thus: The template pBYR11eMa-h6D8-L2 was amplified by PCR with the primers H2-Bam-F and Ext3-R, digested with BamHI and SacI, and ligated with pLITMUS28 (New England Biolabs) digested likewise. A DNA segment encoding the linker “(SGG)₄SGS” (SEQ ID NO. 42) was produced by annealing the oligonucleotides Spe-L-Bam-F and Spe-L-Bam-R, which was ligated with pLIT-BamH2 digested with SpeI and BamHI to make pLIT-L-BamH2.
pKS-HH-gp371 (Kim et al., 2015) containing a DNA sequence encoding the epitope-tagged h6D8 heavy chain was digested with BsaI and SacI to obtain the 467 bp fragment.
pBYR11eMa-h6D8-L2 was digested SacI and XhoI to obtain the 2559 bp fragment containing the Ext 3′ region, Rb7 MAR, SIR, and 35S promoter.
   3. pBYe3R2K2Mc-BAHBcheZE3 (HBche-ZE3 VLP)

A virus-like particle formed by using the Hepatitis B virus core (HBc) with the ZE3 antigen inserted via two linkers into the second of two tandemly-linked HBc copies.

The final construct was assembled by ligation of five fragments:
pBY-R2-GFP (Diamos & Mason, 2018b) was digested XhoI-ClaI to obtain the vector fragment, which contains a single nt mutation at position −3 from the C1 start codon.
The barley alpha amylase signal peptide (BASP) was fused to the N-terminus of the HBc coding sequence, by construction of pLIT-BAHBc. A DNA sequence encoding the 5′ UTR and BASP was amplified from template pBYR2eK2M-6HplcCnetB (Hunter et al., 2019) with primers 35S-F and BASP-G-Bsa-R, and the product digested with XhoI and BsaI to obtain the 146 bp fragment. The HBc coding sequence was amplified by PCR from template pBYR2eK2M-HBche using primers HBc-Bsa-F and HBc176-Sac-R, and the product digested with BsaI and SacI. The two digested PCR products were ligated with pLITMUS28 (New England Biolabs) digested XhoI-SacI to make pLIT-BAHBc, which was digested XhoI-SalI to obtain the 570 bp fragment.
pBYR2eK2M-HBcheL2ic was digested SalI-KpnI to obtain the 311 bp fragment encoding the C-terminal part of the first HBc monomer, the segment linking HBc monomers 1 and 2, and the N-terminal part of the second HBc monomer.
A subclone was made from pBYR2eK2M-HBcheL2ic, named pUC-HBc176iL2c, containing DNA encoding a linker, the L2 antigen, a second linker, and HBc amino acids 78-176. The ZE3 BamHI-SpeI fragment (321 bp) was inserted into the BamHI-SpeI sites of pUC-HBc176iL2c to make pUC-HBc176iZE3, which was digested KpnI-SacI to obtain the 699 bp fragment) encoding an N-terminal linker, ZE3, a C-terminal linker, and HBc amino acids 78-176.
pBYR2e3K2Mc-GFP (Diamos & Mason, 2018a) was digested SacI-ClaI to obtain a 2533 bp segment containing the Ext 3′, 35S 3′, MARc, SIR, and the C2 coding DNA.
   4. pBYRIleM-h6D8-ZEFL62 (ZEFL RIC)

A recombinant immune complex consisting of the h6D8 mAb linked via its heavy chain C-terminus to the Zika virus fusion loop antigen (Zika E E352-S412), with a C-terminal 6D8 epitope tag.

A DNA segment encoding Zika E E352-S412 was amplified by PCR of the E protein plant-optimized gene using primers ZEE62-Bam-F and ZES122-Spe-R, and the product digested with BamHI and SpeI. The resulting 189 bp fragment was inserted to replace the ZE3 sequence in pBYRIleM-h6D8ZE3 at BamHI and SpeI, to produce pBYRIleM-h6D8-ZEFL62.

5. pBYe3R2K2Mc-BAHBcheZE62-122 (HBche-ZEFL VLP)

Similar to the HBche-ZE3 VLP with the Zika virus fusion loop antigen replacing the ZE3 antigen.
The construct was assembled by ligation of fragments:
pBYe3R2K2Mc-BAHBcheZE3 was digested XhoI-SpeI for the vector fragment.
pBYe3R2K2Mc-BAHBcheZE3 was digested XhoI-SalI to obtain the 570 bp fragment.
pBYe3R2K2Mc-BAHBcheZE3 was digested SalI-BamHI to obtain the 337 bp fragment.
pBYR11eM-h6D8-ZEFL62 was digested BamHI-SpeI to obtain the 189 bp fragment encoding Zika E E352-S412.
   ii. Agroinfiltration of Nicotiana benthamiana Leaves

BeYDV plant expression vectors for each construct were introduced into Agrobacterium tumefaciens EHA105 via electroporation. The resulting strains were verified by restriction digestion or PCR, grown overnight at 30 C, and used to infiltrate leaves of 5- to 6-week-old N. benthamiana maintained at 23-25° C. For the RICs, a vector expressing both the ZE3-fused 6D8 heavy chain and the light chain was agroinfiltrated into N. benthamiana leaves. The vector was similar to that previously described in (Kim et al., 2015) with the ZIKV Domain III as the antigen instead of the DENV antigen. Transgenic N. benthamiana plants that have been silenced for xylosyltransferase and fucosyltransferase enzymes were used since these plants produce a highly homogenous, human-like glycosylation pattern that can improve in vivo Fc receptor binding (Castilho and Steinkellner, 2012).

Briefly, the bacteria were pelleted by centrifugation for 8 minutes at 5,000 g and then resuspended in infiltration buffer (10 mM 2-(N-morpholino)ethanesulfonic acid (MES), pH 5.5 and 10 mM MgSO4) to OD₆₀₀=0.2, unless otherwise described. The resulting bacterial suspensions were injected by using a syringe without needle into leaves through a small puncture (Huang et al. 2004). Plant tissue was harvested after 5 days post infiltration (DPI), or as stated for each experiment.

iii. C-RIC Purification

4-5 days post infiltration, the leaves were harvested and extracted in ice-cold, 1:2 w/v buffer at pH 7.4 (100 mM Tris-HCl, 50 mM NaCl, 10 mM EDTA, 0.1% Triton, 10 mg/mL sodium ascorbate, and 0.3 mg/mL PMSF). The remainder of the protein G column chromatography purification protocol was conducted as described.

iv. N-RIC Purification

The infiltration, extraction, and protein G purification for the N-terminal ZE3 RIC was conducted according to the same protocol as described for the C-RIC. The only change was that the extraction buffer used for the N-RIC purification was at pH 9.5 instead of pH 8.

v. ZEFL RIC

The infiltrated leaf material was extracted in ice-cold, 1:2 w/v buffer at pH 9.5 (100 mM Tris-HCl, 50 mM NaCl, 10 mM EDTA, 7M urea, 10 mg/mL sodium ascorbate, and 0.3 mg/mL PMSF). The rest of the protocol was conducted as written in the C-RIC purification. The only change was that the extract was diluted 1:1 in cold, pH 9.5 extraction buffer before loading the extract onto the column for protein G purification. This was done in order to lower the urea concentration so that the ZEFL RIC could bind to the protein G resin. This buffer was the same one used earlier for the extraction process. In addition, at this step, 1M phosphoric acid was added to set the pH of the extract to pH 7.5 in order to allow for optimal binding to the resin.

vi. SDS-PAGE and Western Blot

Samples from the purified RIC elutions were mixed with SDS sample buffer (50 mM Tris-HCl, pH 6.8, 2% SDS, 10% glycerol, 0.02% bromophenol blue) and separated on 4-15% polyacrylamide gels (Bio-Rad). For reducing conditions, 300 mM DTT was added, and the samples were boiled for 10 minutes prior to loading. Polyacrylamide gels were either transferred to a PVDF membrane or stained with Coomassie stain (Bio-Rad) following the manufacturer's instructions. For ZE3 detection, the protein transferred membranes were blocked with 5% dry milk in PBST (PBS with 0.05% tween-20) overnight at 4° C., washed with PBST (3 washes, 5 minutes each), and probed with appropriate antibodies.

The C-RIC samples were detected with goat anti-human IgG (Southern Biotech) antibody that was conjugated with horseradish peroxide (HRP) (Southern Biotech). The N-RIC samples were detected by mouse anti-human IgG (Fc-only) antibody conjugated with HRP (Southern Biotech). Bound antibody was detected with Pierce ECL western Blotting Substrate according to the manufacturer's instructions (ThermoFisher Scientific).

vii. HBche-ZE3 VLP

The vector containing the HBche-ZE3 VLP construct was agroinfiltrated into the leaves of 4-6-week-old N. benthamiana plants. At 4-5 DPI, the leaves were harvested, extracted in ice-cold, 1:2 w/v buffer at pH 7.4 (100 mM Tris-HCl, 50 mM NaCl, 10 mM EDTA, 0.1% Triton, 10 mg/mL sodium ascorbate, and 0.3 mg/mL PMSF), and analyzed by sucrose gradient sedimentation.

viii. Electron Microscopy

To obtain the images of the HBche-ZE3 VLP, samples of HBche-ZE3 VLP partially purified by sucrose gradient sedimentation were initially incubated on 75/300 mesh grids coated with formvar. After incubation, the samples were washed twice with deionized water then negatively stained with 2% aqueous uranyl acetate. The transmission electron microscopy was performed with a Phillips CM-12 microscope. The images were acquired with a Gatan model 791 CCD camera.

ix. HBche-ZEFL VLP

Through agroinfiltration, the vector containing the HBche-ZEFL VLP was delivered into the leaves of 4- to 6-week-old N. benthamiana plants. At 4-5 DPI, the leaves were harvested, extracted in ice-cold, 1:2 w/v buffer at pH 7.4 (100 mM Tris-HCl, 50 mM NaCl, 10 mM EDTA, 0.1% Triton, 10 mg/mL sodium ascorbate, and 0.3 mg/mL PMSF), and analyzed by sucrose gradient sedimentation. After the sucrose gradient sedimentation, samples of the sucrose fractions were run on a Coomassie-stained gel. Upon reducing conditions, a band at the appropriate size was visible (FIG. 22A).

x. Immunization of Mice and Sample Collection

Female BALB/C mice (6-8 weeks old,) were immunized subcutaneously with the following constructs: purified plant-expressed ZE3 N-terminal RIC (N-RIC), ZE3 C-terminal RIC (C-RIC), HBche-ZE3 VLP, ZEFL RIC, and ZEFL HBche-VLP. The antigens were either delivered alone or in various combinations of RIC and VLP mixed at a ratio of 1:1. Most groups were given antigen mixed 1:1 with Imject Alum (Thermo Scientific, Rockford, IL) prior to immunization. However, two groups, HBche-ZE3 alone and the HBche-ZE3+C-RIC, were not given alum as an adjuvant in order to test the effect of alum on the response elicited by the delivered vaccine antigens. All groups contained 6 mice. For the ZE3-containing groups, the total dose of antigen was set to deliver an equivalent 4 μg of ZE3. For the ZEFL-containing groups, the total dose of antigen was 2 μg. Doses were given on days 0, 28, and 56. Serum collection was done as described (Santi et al., 2008) by submandibular bleed on days 0, 28, and 56, and 86. All animals were handled in accordance to the Animal Welfare Act and Arizona State University IACUC.

xi. Antibody Measurements

Mouse antibody titers were measured by ELISA. Zika soluble ectodomain E (ZsE) protein (amino acids 291-693, 6-His tagged) was produced by agro-infiltration delivery of expression vector pBYe3R2K2Mc-BAZsE6H into leaves of N. benthamiana and purified by metal affinity chromatography. ZsE was bound to 96-well high-binding polystyrene plates (Corning). After the plates were blocked with 5% nonfat dry milk in PBST (PBS with 0.05% tween-20), the wells were washed with PBST. Then, the diluted mouse sera from the terminal bleed were added, and the plate incubated at 37 C for 1 hour. Mouse antibodies were detected by incubation with polyclonal goat anti-mouse IgG-horseradish peroxidase conjugate (Sigma). The plate was developed with TMB substrate (Pierce) and the absorbance was read at 450 nm. Endpoint titers were taken as the reciprocal of the lowest dilution which produced an OD450 reading twice the background. IgG1 and IgG2a antibodies were measured with goat-anti mouse IgG1 or IgG2a horseradish peroxidase conjugate (Southern Biotech). Statistical analysis and determination of signification between the various vaccine treatment was done by non-parametric Mann-Whitney tests using GraphPad prism software. The results, which are displayed in FIGS. 5 and 6, show the endpoint titers after the second and after the terminal bleed.

b. C-RIC, N-RIC production

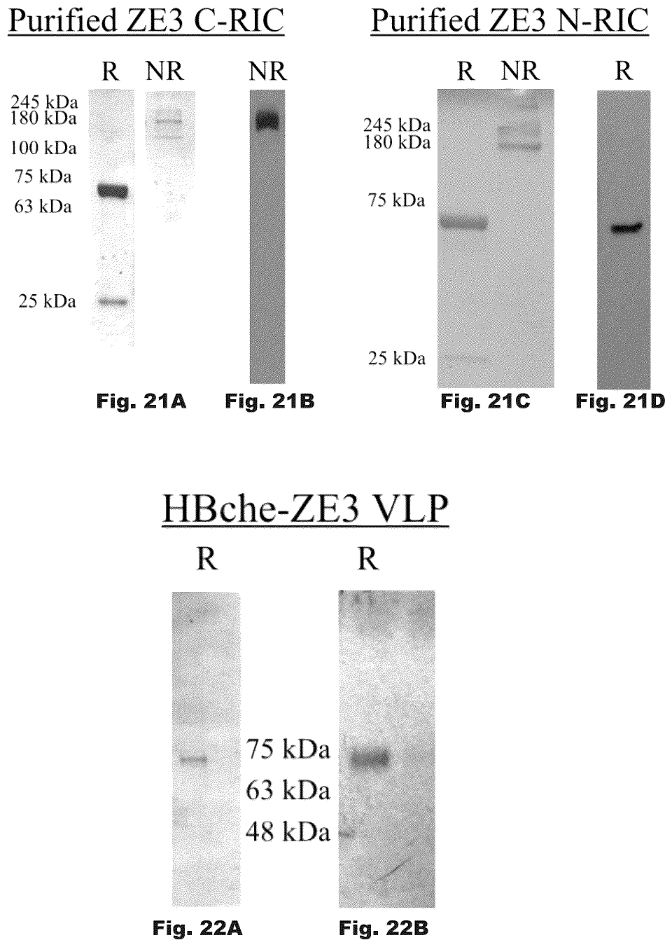

After purification by protein G affinity, C—RIC and N-RIC samples were examined by SDS-PAGE and western blot. Staining with Coomassie Brilliant Blue showd the C-RIC sample was highly pure and that the protein displayed expected molecular mass under nonreducing conditions (˜178 kDa) and reducing conditions (˜65.4 kDa and 25 kDa bands) (FIG. 21A). A western blot, probed for the presence of IgG, also showed bands at the appropriate size (FIG. 21B).

Likewise, a Coomassie-stained gel containing samples of the purified N-RIC material showed appropriately sized bands under both reducing and non-reducing conditions that signified correctly assembled RICs (FIG. 21C). Under reducing conditions, the expected band sizes were ˜65.4 kDa and 25 kDa bands. A fully-formed RIC was expected to show a band around 178 kDa. A western blot of the purified samples that was probed for the antibody Fc region also showed bands at the expected size (FIG. 21D).

c. HBche-ZE3 VLP Production

Samples of the sucrose gradient preparations were run on a Coomassie-stained gel. Upon reducing conditions, bands at the appropriate size were visible (FIG. 22A). A western blot with the HBche-ZE3 VLP was probed with a rabbit polyclonal anti-Zika virus envelope protein antibody (GeneTex) and detected by goat anti rabbit plus HRP (SeraCare) (FIG. 22B). To show formation of virus-like particles, the sucrose gradient fractions were further analyzed by electron microscopy (FIG. 24). The sucrose gradient fractions containing the target VLP were combined and dialyzed against 1×PBS for use in the mouse immunization trials.

d. ZEFL-RIC and ZEFL VLP Production

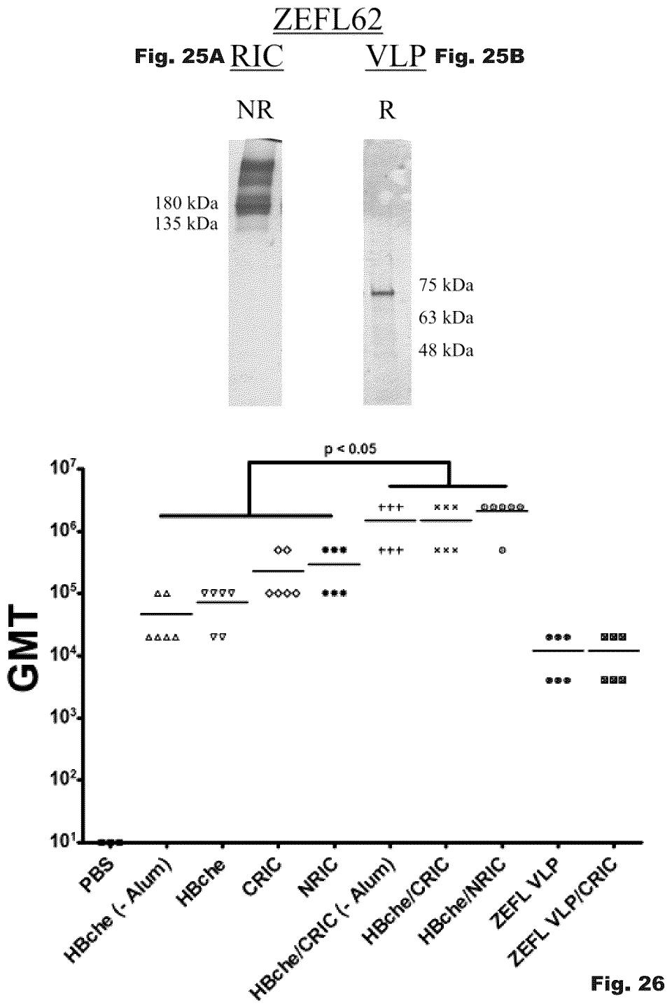

The ZEFL-RIC and ZEFL VLP were purified and examined by SDS-PAGE with Coomassie staining (FIG. 25). Expected molecular masses for nonreduced ZEFL-RIC and reduced ZEFL VLP were observed.

e. Mouse Immunization Study

BALB/c mice (6 per group) were immunized subcutaneously with ZE3 N-terminal RIC (N-RIC), ZE3 C-terminal RIC (C-RIC), HBche-ZE3 VLP (HBche-ZE3), ZEFL RIC, and ZEFL HBche-VLP either alone or in various combinations of RIC and VLP mixed 1:1. After the second dose (FIG. 26), the combination groups of HBche-ZE3+C-RIC delivered either with or without alum adjuvant had roughly a 14-fold increase in anti-ZE3 IgG antibody titers when compared to the groups containing the HBche-ZE3, delivered with or without adjuvant. In addition, there was a 6-fold increase in antibody titers of the VLP/RIC combination groups compared to the groups containing either C-RIC or N-RIC alone. These data show that co-delivery of the RIC and VLP produced substantially increased antibody titers when compared to delivery of either VLP alone or RIC alone. Furthermore, since there was an insignificant difference between the HBche-ZE3 VLP groups and the HBche-ZE3 VLP+C-RIC groups that were delivered with or without adjuvant, the use of alum adjuvant does not seem to affect the synergistic effect seen by the co-delivery of the VLP and RIC. The C-RIC and N-RIC groups had similar titers with no statistically significant difference.

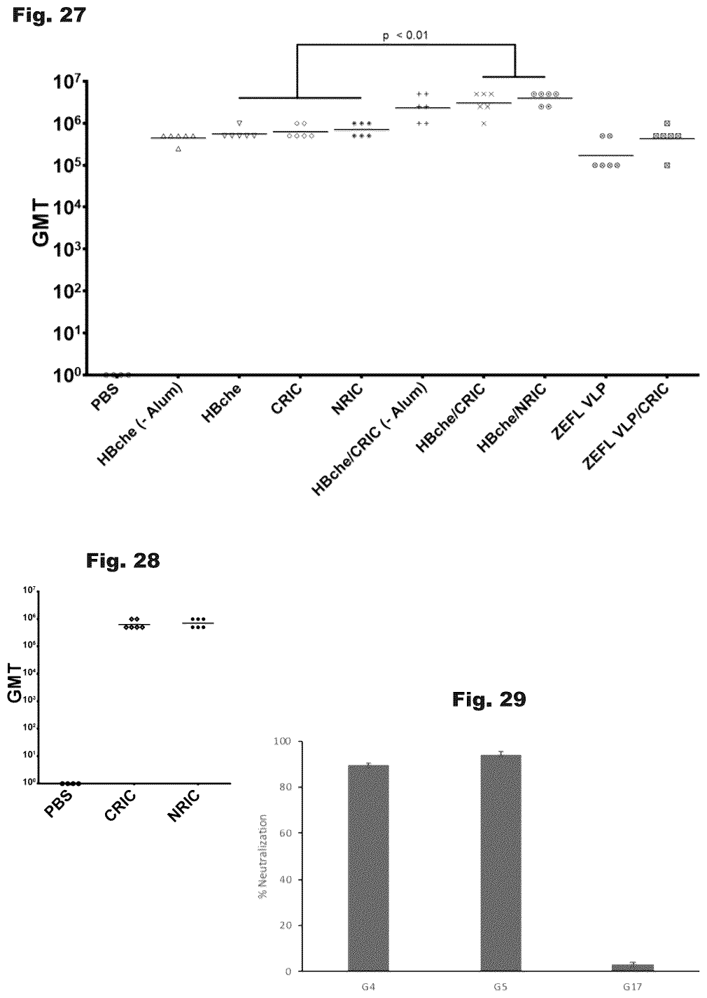

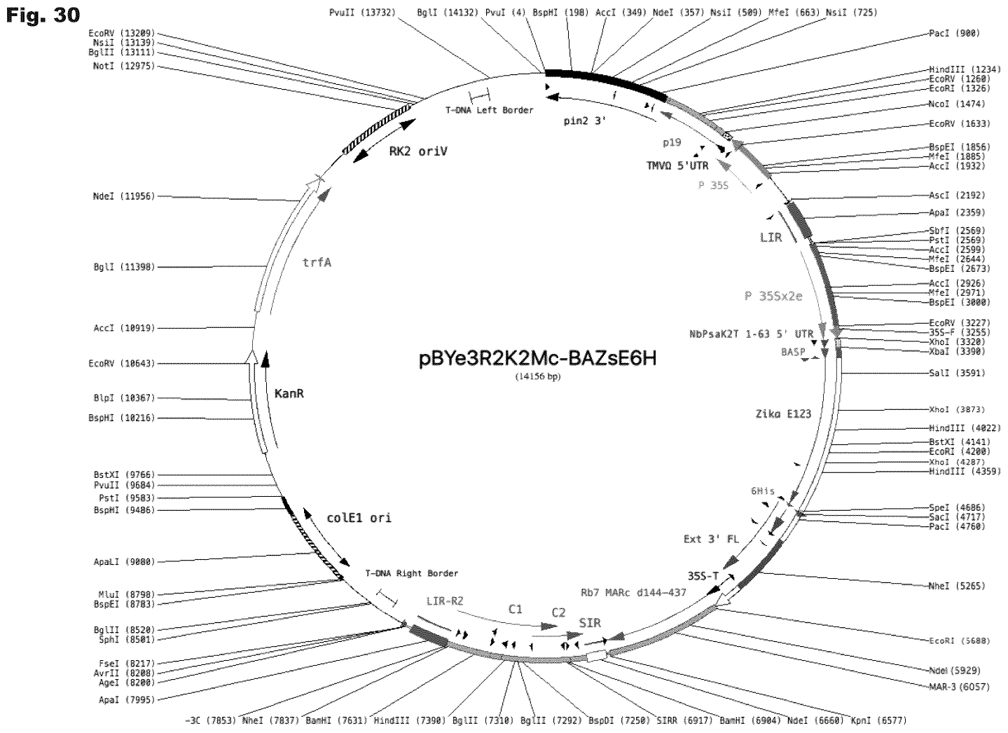

These trends continued with the antibody titers measured after the third dose (FIG. 27, Table 4 lists the mean titer counts for each treatment group). Once again, both the C-RIC and N—RIC groups showed highly similar antibody titers. Delivery of either RIC resulted in mean endpoint antibody titers that were greater than 100,000. However, the co-delivery of either C-RIC or N-RIC with the HBche-ZE3 VLP produced increased antibody titers greater than 1,000,000, a 10-fold increase when compared to the RIC-only groups. This strongly suggests that there is a synergistic effect seen when the HBche-ZE3 is co-delivered with either the C-RIC or N-RIC. The antibody titers of the groups without alum were slightly lower than the corresponding groups that were delivered with alum. However, this difference was not statistically significant.

TABLE 4
Mean titer after three doses.

| Group | Mean titer |
|---|---|
| PBS | 0 |
| HBc | 600,000 |
| HBche-alum | 450,000 |
| HBche | 550,000 |
| CRIC | 700,000 |
| NRIC | 750,000 |
| HBc/CRIC | 3,500,000 |
| HBche/CRIC-alum | 2,800,000 |
| HBche/CRIC | 3,500,000 |
| HBche/NRIC | 4,000,000 |
| zE-Fc: | 7,000,000 |
| FL VLP | 250,000 |
| FL VLP/RIC | 500,000 |

The ZEFL-containing groups performed equally well after dose 2 with no statistical significance seen between the titers elicited by either the ZEFL VLP alone or the ZEFL VLP+ZEFL RIC group. After dose three, a slight but statistically insignificant increase in antibody titers was observed in the combination group.

### REFERENCES CITED

(NIAID), N.I. of A. and I.D. VRC 705: A Zika Virus DNA Vaccine in Healthy Adults and Adolescents (DNA).
Alam, A., Jiang, L., Kittleson, G. A., Steadman, K. D., Nandi, S., Fuqua, J. L., Palmer, K. E., Tuse, D., and McDonald, K. A. (2018). Technoeconomic Modeling of Plant-Based Griffithsin Manufacturing. Front. Bioeng. Biotechnol. 6, 102.
Ali, S. T., Cowling, B. J., Lau, E. H., Fang, V. J., & Leung, G. M. (2018). Mitigation of Influenza B Epidemic with School Closures, Hong Kong, 2018. Emerging infectious diseases, 24(11), 2071.
Alphs, H. H., Gambhira, R., Karanam, B., Roberts, J. N., Jagu, S., Schiller, J. T., et al. (2008). Protection against heterologous human papillomavirus challenge by a synthetic lipopeptide vaccine containing a broadly cross-neutralizing epitope of L2. Proc. Natl. Acad. Sci. U.S.A 105, 5850-5. doi:10.1073/pnas.0800868105.
Atsmon, J., Kate-Ilovitz, E., Shaikevich, D., Singer, Y., Volokhov, I., Haim, K. Y., & Ben-Yedidia, T. (2012). Safety and immunogenicity of multimeric-001-a novel universal influenza vaccine. Journal of clinical immunology, 32(3), 595-603.
Avalos, A. M., & Ploegh, H. (2014). Early BCR events and antigen capture, processing, and loading on MHC class II on B cells. Frontiers in immunology, 5, 92.
Bajtay, Z., Csomor, E., Sándor, N., and Erdei, A. (2006). Expression and role of Fc- and complement-receptors on human dendritic cells. in Immunology Letters, 46-52. doi:10.1016/j.imlet.2005.11.023.
Barzon, L., and Palù, G. (2017). Current views on Zika virus vaccine development. Expert Opin. Biol. Ther. 17, 1185-1192.
Belmusto-Worn, V. E., Sanchez, J. L., Mccarthy, K., Nichols, R., Bautista, C. T., Magill, A. J., Pastor-Cauna, G., Echevarria, C., Laguna-Torres, V. A., Samame, B. K., et al. (2005). RANDOMIZED, DOUBLE-BLIND, PHASE III, PIVOTAL FIELD TRIAL OF THE COMPARATIVE IMMUNOGENICITY, SAFETY, AND TOLERABILITY OF TWO YELLOW FEVER 17D VACCINES (ARILVAX™ AND YF-VAX) IN HEALTHY INFANTS AND CHILDREN IN PERU.
Bianchi, E., Liang, X., Ingallinella, P., Finotto, M., Chastain, M. A., Fan, J., . . . & Manger, W. (2005). Universal influenza B vaccine based on the maturational cleavage site of the hemagglutinin precursor. Journal of virology, 79(12), 7380-7388.
Blokhina, E. A., Kuprianov, V. V., Stepanova, L. A., Tsybalova, L. M., Kiselev, O. I., Ravin, N. V., & Skryabin, K. G. (2013). A molecular assembly system for presentation of antigens on the surface of HBc virus-like particles. Virology, 435(2), 293-300.
Boigard, H., Alimova, A., Martin, G. R., Katz, A., Gottlieb, P., and Galarza, J. M. (2017). Zika virus-like particle (VLP) based vaccine. PLoS Negl. Trop. Dis. 11, e0005608.
Bresee, J., Fitzner, J., Campbell, H., Cohen, C., Cozza, V., Jara, J., . . . & Lee, V. (2018). Progress and remaining gaps in estimating the global disease burden of influenza. Emerging infectious diseases, 24(7), 1173.
Brown, A. L., Francis, M. J., Hastings, G. Z., Parry, N. R., Barnett, P. V, Rowlands, D. J., et al. (1991). Foreign epitopes in immunodominant regions of hepatitis B core particles are highly immunogenic and conformationally restricted. Vaccine 9, 595-601.
Brown, D. R., Kjaer, S. K., Sigurdsson, K., Iversen, O., Hernandez-Avila, M., Wheeler, C. M., et al. (2009). The Impact of Quadrivalent Human Papillomavirus (HPV; Types 6, 11, 16, and 18) L1 Virus-Like Particle Vaccine on Infection and Disease Due to Oncogenic Nonvaccine HPV Types in Generally HPV-Naive Women Aged 16-26 Years. J. Infect. Dis. 199, 926-935. doi:10.1086/597307.
Burns, A., Van der Mensbrugghe, D., & Timmer, H. (2006). Evaluating the economic consequences of avian influenza. World Bank.
Caini, S., Alonso, W. J., Balmaseda, A., Bruno, A., Bustos, P., Castillo, L., . . . & Kusznierz, G. F. (2017). Characteristics of seasonal influenza A and B in Latin America: Influenza surveillance data from ten countries. PloS one, 12(3), e0174592.
Castilho A, Steinkellner H. Glyco-engineering in plants to produce human-like N-glycan structures. Biotechnol J 2012; 7:1088-98. doi:10.1002/biot.201200032.
Centers for Disease Control and Prevention. (2018, November 02). Summary of the 2017-2018 Influenza Season.
Chackerian, B. (2007). Virus-like particles: flexible platforms for vaccine development. Expert review of vaccines, 6(3), 381-390.
Chargelegue, D., Drake, P. M. W., Obregon, P., Prada, A., Fairweather, N., and Ma, J. K. C. (2005). Highly immunogenic and protective recombinant vaccine candidate expressed in transgenic plants. Infect. Immun. 73, 5915-5922. doi:10.1128/IAI.73.9.5915-5922.2005.
Chen Q, Davis K R. The potential of plants as a system for the development and production of human biologics. F1000Research 2016; 5:912. doi:10.12688/f1000research.8010.1.
Chen, Q., He, J., Phoolcharoen, W., & Mason, H. S. (2011). Geminiviral vectors based on bean yellow dwarf virus for production of vaccine antigens and monoclonal antibodies in plants. Human vaccines, 7(3), 331-338.
Cooper, A., Tal, G., Lider, O., & Shaul, Y. (2005). Cytokine induction by the hepatitis B virus capsid in macrophages is facilitated by membrane heparan sulfate and involves TLR2. The Journal of Immunology, 175(5), 3165-3176.
Coutelier, J. P., van der Logt, J. T., Heessen, F. W., Vink, A., and van Snick, J. (1988). Virally induced modulation of murine IgG antibody subclasses. J Exp Med 168, 2373-2378. doi:10.1084/jem.168.6.2373.
Crow, J. M. (2012). HPV: The global burden. Nature 488, S2-S3. doi:10.1038/488S2a.
Dai, L., Song, J., Lu, X., Deng, Y.-Q., Musyoki, A. M., Cheng, H., Zhang, Y., Yuan, Y., Song, H., Haywood, J., et al. (2016). Structures of the Zika Virus Envelope Protein and Its Complex with a Flavivirus Broadly Protective Antibody. Cell Host Microbe 19, 696-704.
Davies, J. W., & Stanley, J. (1989). Geminivirus genes and vectors. Trends in Genetics, 5, 77-81.
de Jong, J. M. H., Schuurhuis, D. H., Ioan-Facsinay, A., van der Voort, E. I. H., Huizinga, T. W. J., Ossendorp, F., et al. (2006). Murine Fc receptors for IgG are redundant in facilitating presentation of immune complex derived antigen to CD8+ T cells in vivo. Mol. Immunol. 43, 2045-2050. doi:10.1016/j.molimm.2006.01.002.
Diamos A G, Mason H S (2018) Chimeric 3′ Flanking Regions Strongly Enhance Gene Expression in Plants. Plant Biotechnol J., 2018 Apr. 10. doi: 10.1111/pbi.12931. [Epub ahead of print] PMID: 29637682.
Diamos A G, Mason H S (2018). Modifying the replication of geminiviral vectors reduces cell death and enhances expression of biopharmaceutical proteins in Nicotiana benthamiana leaves. Front Plant Sci. 9:1974. doi: 10.3389/fpls.2018.01974. eCollection 2018. PMID: 30687368
Diamos, A. G., & Mason, H. S. (2018). High-level expression and enrichment of norovirus virus-like particles in plants using modified geminiviral vectors. Protein expression and purification.
Diamos, A. G., Rosenthal, S. H., & Mason, H. S. (2016). 5′ and 3′ untranslated regions strongly enhance performance of geminiviral replicons in Nicotiana benthamiana leaves. Frontiers in plant science, 7, 200.
Diamos, A. G., Larios, D., Brown, L., Kilbourne, J., Kim, H. S., Saxena, D., Palmer, K. E., and Mason, H. S. (2019). Vaccine synergy with virus-like particle and immune complex platforms for delivery of human papillomavirus L2 antigen. 37, 137-144.
Doorbar, J., Egawa, N., Griffin, H., Kranjec, C., and Murakami, I. (2015). Human papillomavirus molecular biology and disease association. Rev. Med. Virol. 25, 2-23. doi:10.1002/rmv.1822.
Dreyfus, C., Laursen, N. S., Kwaks, T., Zuijdgeest, D., Khayat, R., Ekiert, D. C., . . . & van der Vlugt, R. (2012). Highly conserved protective epitopes on influenza B viruses. Science, 337(6100), 1343-1348.
Durbin, A., and Wilder-Smith, A. (2017). An update on Zika vaccine developments. Expert Rev. Vaccines 16, 781-787.
Ebrahimi, S. M., Dabaghian, M., Tebianian, M., & Jazi, M. H. Z. (2012). In contrast to conventional inactivated influenza vaccines, 4×M2e. HSP70c fusion protein fully protected mice against lethal dose of H1, H3 and H9 influenza A isolates circulating in Iran. Virology, 430(1), 63-72.
Eichelberger, M. C., Morens, D. M., & Taubenberger, J. K. (2018). Neuraminidase as an influenza vaccine antigen: a low hanging fruit, ready for picking to improve vaccine effectiveness. Current opinion in immunology, 53, 38-44.
Eisenberg, R. (1976). The specificity and polyvalency of binding of a monoclonal rheumatoid factor. Immunochemistry, 13(4), 355-359.
Eisfeld, A. J., Neumann, G., & Kawaoka, Y. (2015). At the centre: influenza A virus ribonucleoproteins. Nature Reviews Microbiology, 13(1), 28.
El Bakkouri, K., Descamps, F., De Filette, M., Smet, A., Festjens, E., Birkett, A., . . . & Saelens, X. (2011). Universal vaccine based on ectodomain of matrix protein 2 of influenza A: Fc receptors and alveolar macrophages mediate protection. The Journal of Immunology, 186(2), 1022-1031.
Eliasson, D. G., Omokanye, A., Schon, K., Wenzel, U. A., Bernasconi, V., Bemark, M., . . . & Fiers, W. (2018). M2e-tetramer-specific memory CD4 T cells are broadly protective against influenza infection. Mucosal immunology, 11(1), 273.
Ellebedy, A. H., Krammer, F., Li, G. M., Miller, M. S., Chiu, C., Wrammert, J., . . . & Edupuganti, S. (2014). Induction of broadly cross-reactive antibody responses to the influenza HA stem region following H5N1 vaccination in humans. Proceedings of the National Academy of Sciences, 111(36), 13133-13138.
Favre, B. C. (2018). The Development of a Plant-Expressed M2e-Based Universal Influenza Vaccine (Honors thesis). Retrieved from the Barrett, The Honors College Thesis/Creative Project Collection.
Fiers, W., De Filette, M., Birkett, A., Neirynck, S., & Jou, W. M. (2004). A “universal” human influenza A vaccine. Virus research, 103(1), 173-176.
Fiers, W., De Filette, M., El Bakkouri, K., Schepens, B., Roose, K., Schotsaert, M., . . . & Saelens, X. (2009). M2e-based universal influenza A vaccine. Vaccine, 27(45), 6280-6283.
Fischer, R., & Emans, N. (2000). Molecular farming of pharmaceutical proteins. Transgenic research, 9(4-5), 279-299.
Flannery, B., Chung, J. R., Thaker, S. N., Monto, A. S., Martin, E. T., Belongia, E. A., . . . & Nowalk, M. P. (2017). Interim estimates of 2016-17 seasonal influenza vaccine effectiveness-United States, February 2017. MMWR. Morbidity and mortality weekly report, 66(6), 167.
Flannery, B., Clippard, J., Zimmerman, R. K., Nowalk, M. P., Jackson, M. L., Jackson, L. A., . . . & Gaglani, M. (2015). Early estimates of seasonal influenza vaccine effectiveness-United States, January 2015. MMWR. Morbidity and mortality weekly report, 64(1), 10.
Flannery, B., Thaker, S. N., Clippard, J., Monto, A. S., Ohmit, S. E., Zimmerman, R. K., . . . & Belongia, E. A. (2014). Interim estimates of 2013-14 seasonal influenza vaccine effectiveness-United States, February 2014. Morbidity and Mortality Weekly Report, 63(7), 137-142.
Fridman, W. H. (1991). Fc receptors and immunoglobulin binding factors. The FASEB journal, 5(12), 2684-2690.
allie, D. R. (2002). The 5′-leader of tobacco mosaic virus promotes translation through enhanced recruitment of eIF4F. Nucleic acids research, 30(15), 3401-3411.
Gambhira, R., Jagu, S., Karanam, B., Gravitt, P. E., Culp, T. D., Christensen, N. D., et al. (2007). Protection of Rabbits against Challenge with Rabbit Papillomaviruses by Immunization with the N Terminus of Human Papillomavirus Type 16 Minor Capsid Antigen L2. J. Virol. 81, 11585-11592. doi:10.1128/JVI.01577-07.
Gambhira, R., Karanam, B., Jagu, S., Roberts, J. N., Buck, C. B., Bossis, I., et al. (2007). A protective and broadly cross-neutralizing epitope of human papillomavirus L2. J. Virol. 81, 13927-31. doi:10.1128/JVI.00936-07.
Gaukroger, J. M., Chandrachud, L. M., O'Neil, B. W., Grindlay, G. J., Knowles, G., and Campo, M. S. (1996). Vaccination of cattle with bovine papillomavirus type 4 L2 elicits the production of virus-neutralizing antibodies. J. Gen. Virol. 77, 1577-1583. doi:10.1099/0022-1317-77-7-1577.
Gerhard, W., Mozdzanowska, K., Furchner, M., Washko, G., and Maiese, K. (1997). Role of the B-cell response in recovery of mice from primary influenza virus infection. Immunol. Rev. 159, 95-103. doi:10.1111/j.1600-065X.1997.tb01009.x.
Haiyan Zhao, A., Fernandez, E., Dowd, K. A., Pierson, T. C., Diamond, M. S., Fremont, D. H., Zhao, H., Speer, S. D., Platt, D. J., Gorman, M. J., et al. (2016). Structural Basis of Zika Virus-Specific Antibody Protection Accession Numbers 5KVD 5KVE 5KVF 5KVG Article Structural Basis of Zika Virus-Specific Antibody Protection. Cell 166.
Halweg, C., Thompson, W. F., & Spiker, S. (2005). The Rb7 matrix attachment region increases the likelihood and magnitude of transgene expression in tobacco cells: a flow cytometric study. The Plant Cell, 17(2), 418-429.
Hause, B. M., Collin, E. A., Liu, R., Huang, B., Sheng, Z., Lu, W., . . . & Li, F. (2014). Characterization of a novel influenza virus in cattle and swine: proposal for a new genus in the Orthomyxoviridae family. MBio, 5(2), e00031-14.
Hay, A. J., Gregory, V., Douglas, A. R., & Lin, Y. P. (2001). The evolution of human influenza viruses. Philosophical Transactions of the Royal Society of London. Series B, 356(1416), 1861.
Hefferon, K. L. (2014). DNA virus vectors for vaccine production in plants: spotlight on geminiviruses. Vaccines, 2(3), 642-653.
Heinz, F. X., Holzmann, H., Essl, A., and Kundi, M. (2007). Field effectiveness of vaccination against tick-borne encephalitis. Vaccine 25, 7559-7567.
Hiatt, A., Zeitlin, L., and Whaley, K. J. (2014). Plant-Derived Monoclonal Antibodies for Prevention and Treatment of Infectious Disease. Microbiol. Spectr. 2. doi:10.1128/microbiolspec.AID-0004-2012.
Hioe, C. E., Visciano, M. L., Kumar, R., Liu, J., Mack, E. A., Simon, R. E., et al. (2009). The use of immune complex vaccines to enhance antibody responses against neutralizing epitopes on HIV-1 envelope gp120. Vaccine 28, 352-360. doi:10.1016/j.vaccine.2009.10.040.
Huang, Z., & Mason, H. S. (2004). Conformational analysis of hepatitis B surface antigen fusions in an Agrobacterium-mediated transient expression system. Plant Biotechnology Journal, 2(3), 241-249.
Huang, Z., Chen, Q., Hjelm, B., Arntzen, C., and Mason, H. (2009). A DNA replicon system for rapid high-level production of virus-like particles in plants. Biotechnol. Bioeng. 103, 706-714. doi:10.1002/bit.22299.
Huang, Z., Phoolcharoen, W., Lai, H., Piensook, K., Cardineau, G., Zeitlin, L., et al. (2010). High-level rapid production of full-size monoclonal antibodies in plants by a single-vector DNA replicon system. Biotechnol. Bioeng. 106, 9-17. doi:10.1002/bit.22652.
Huber, V. C., McKeon, R. M., Brackin, M. N., Miller, L. A., Keating, R., Brown, S. A., . . . & McCullers, J. A. (2006). Distinct contributions of vaccine-induced immunoglobulin G1 (IgG1) and IgG2a antibodies to protective immunity against influenza. Clin. Vaccine Immunol., 13(9), 981-990.
Ingle, N. B., Virkar, R. G., & Arankalle, V. A. (2017). Inter-Clade Protection Offered by Mw-Adjuvanted Recombinant HA, NP Proteins, and M2e Peptide Combination Vaccine in Mice Correlates with Cellular Immune Response. Frontiers in immunology, 7, 674.
Inglis, S. C., Carroll, A. R., Lamb, R. A., & Mahy, B. W. (1976). Polypeptides specified by the influenza virus genome: I. Evidence for eight distinct gene products specified by fowl plague virus. Virology, 74(2), 489-503.
Iuliano, A. D., Roguski, K. M., Chang, H. H., Muscatello, D. J., Palekar, R., Tempia, S., . . . & Wu, P. (2018). Estimates of global seasonal influenza-associated respiratory mortality: a modelling study. The Lancet, 391(10127), 1285-1300.
Jackson, L., Jackson, M. L., Phillips, C. H., Benoit, J., Belongia, E. A., Cole, D., . . . & Strey, S. K. (2013). Interim adjusted estimates of seasonal influenza vaccine effectiveness-United States, February 2013.
Jackson, M. L., Chung, J. R., Jackson, L. A., Phillips, C. H., Benoit, J., Monto, A. S., . . . & Murthy, K. (2017). Influenza vaccine effectiveness in the United States during the 2015-2016 season. New England Journal of Medicine, 377(6), 534-543.
Jackson, M. L., Phillips, C. H., Benoit, J., Jackson, L. A., Gaglani, M., Murthy, K., . . . & Flannery, B. (2018). Burden of medically attended influenza infection and cases averted by vaccination-United States, 2013/14 through 2015/16 influenza seasons. Vaccine, 36(4), 467-472.
Jefferis, R. (2009). Glycosylation as a strategy to improve antibody-based therapeutics. Nat. Rev. Drug Discov. 8, 226-234. doi:10.1038/nrd2804.
Kanda, Y., Yamada, T., Mori, K., Okazaki, A., Inoue, M., Kitajima-Miyama, K., et al. (2007). Comparison of biological activity among nonfucosylated therapeutic IgG1 antibodies with three different N-linked Fc oligosaccharides: The high-mannose, hybrid, and complex types. Glycobiology 17, 104-118. doi:10.1093/glycob/cw1057.
Kawana, K., Yoshikawa, H., Taketani, Y., Yoshiike, K., and Kanda, T. (1999). Common neutralization epitope in minor capsid protein L2 of human papillomavirus types 16 and 6. J. Virol. 73, 6188-90.
Kim, K. H., Kwon, Y. M., Lee, Y. T., Kim, M. C., Hwang, H., Ko, E. J., . . . & Kang, S. M. (2018). Virus-Like Particles Are a Superior Platform for Presenting M2e Epitopes to Prime Humoral and Cellular Immunity against Influenza Virus. Vaccines, 6(4), 66.
Kim, M. Y., Reljic, R., Kilbourne, J., Ceballos-Olvera, I., Yang, M. S., Reyes-del Valle, J., & Mason, H. S. (2015). Novel vaccination approach for dengue infection based on recombinant immune complex universal platform. Vaccine, 33(15), 1830-1838.
Kines, R. C., Thompson, C. D., Lowy, D. R., Schiller, J. T., and Day, P. M. (2009). The initial steps leading to papillomavirus infection occur on the basement membrane prior to cell surface binding. Proc. Natl. Acad. Sci. 106, 20458-20463. doi:10.1073/pnas.0908502106.
Kirnbauer, R., Booyt, F., Chengt, N., Lowy, D. R., and Schiller, J. T. (1992). Papillomavirus Li major capsid protein self-assembles into virus-like particles that are highly immunogenic. Med. Sci. 89, 12180-12184. doi:10.1073/pnas.89.24.12180.
Kolpe, A., Schepens, B., Ye, L., Staeheli, P., & Saelens, X. (2018). Passively transferred M2e-specific monoclonal antibody reduces influenza A virus transmission in mice. Antiviral research, 158, 244-254.
Kondo, K., Ishii, Y., Ochi, H., Matsumoto, T., Yoshikawa, H., and Kanda, T. (2007). Neutralization of HPV16, 18, 31, and 58 pseudovirions with antisera induced by immunizing rabbits with synthetic peptides representing segments of the HPV16 minor capsid protein L2 surface region. Virology 358, 266-272. doi:10.1016/j.virol.2006.08.037.
Kondo, K., Ochi, H., Matsumoto, T., Yoshikawa, H., and Kanda, T. (2008). Modification of human papillomavirus-like particle vaccine by insertion of the cross-reactive L2-epitopes. J. Med. Virol. 80, 841-846. doi:10.1002/jmv.21124.
Kosik, I., Angeletti, D., Gibbs, J. S., Angel, M., Takeda, K., Kosikova, M., . . . & Yewdell, J. W. (2019). Neuraminidase inhibition contributes to influenza A virus neutralization by anti-hemagglutinin stem antibodies. Journal of Experimental Medicine, 216(2), 304-316.
Krammer, F., & Palese, P. (2019). Universal influenza virus vaccines that target the conserved hemagglutinin stalk and conserved sites in the head domain. The Journal of infectious diseases.
Krieger, G., Kneba, M., Bolz, I., Volling, P., Wessels, J., and Nagel, G. A. (1985). Binding characteristics of three complement dependent assays for the detection of immune complexes in human serum. J. Clin. Lab. Immunol. 18, 129-134.
Krishnavajhala, H. R., Williams, J., & Heidner, H. (2018). An influenza A virus vaccine based on an M2e-modified alphavirus. Archives of virology, 163(2), 483-488.
Lamb, R. A. (1983). The influenza virus RNA segments and their encoded proteins. In Genetics of influenza viruses (pp. 21-69). Springer, Vienna.
Lamb, R. A., Zebedee, S. L., & Richardson, C. D. (1985). Influenza virus M2 protein is an integral membrane protein expressed on the infected-cell surface. Cell, 40(3), 627-633.
Lazarowitz, S. G., & Shepherd, R. J. (1992). Geminiviruses: genome structure and gene function. Critical Reviews in Plant Sciences, 11(4), 327-349.
Lee, S. Y., Kang, J. O., & Chang, J. (2019). Nucleoprotein vaccine induces cross-protective cytotoxic T lymphocytes against both lineages of influenza B virus. Clinical and Experimental Vaccine Research, 8(1), 54-63.
Liu, W., Zou, P., Ding, J., Lu, Y., & Chen, Y. H. (2005). Sequence comparison between the extracellular domain of M2 protein human and avian influenza A virus provides new information for bivalent influenza vaccine design. Microbes and infection, 7(2), 171-177.
Mardanova, E. S., & Ravin, N. V. (2018). Plant-produced Recombinant Influenza A Vaccines Based on the M2e Peptide. Current pharmaceutical design, 24(12), 1317-1324.
Mardanova, E. S., Kotlyarov, R. Y., Kuprianov, V. V., Stepanova, L. A., Tsybalova, L. M., Lomonossoff, G. P., & Ravin, N. V. (2015). High immunogenicity of plant-produced influenza based on the M2e peptide fused to flagellin. Biotechnology, 15(42), 25.
Mariani, L., and Venuti, A. (2010). HPV vaccine: an overview of immune response, clinical protection, and new approaches for the future. J. Transl. Med. 8, 105. doi:10.1186/1479-5876-8-105.
Markine-Goriaynoff, D., and Coutelier, J.-P. (2002). Increased Efficacy of the Immunoglobulin G2a Subclass in Antibody-Mediated Protection against Lactate Dehydrogenase-Elevating Virus-Induced Polioencephalomyelitis Revealed with Switch Mutants. J. Virol. 76, 432-435. doi:10.1128/JVI.76.1.432-435.2002.
Marusic, C., Pioli, C., Stelter, S., Novelli, F., Lonoce, C., Morrocchi, E., et al. (2017). N-glycan engineering of a plant-produced anti-CD20-hIL-2 immunocytokine significantly enhances its effector functions. Biotechnol. Bioeng. 115, 565-576. doi:10.1002/bit.26503.
Mason, H. S. (2016). Recombinant immune complexes as versatile and potent vaccines. Hum. Vaccines Immunother. 12, 988-989. doi:10.1080/21645515.2015.1116655.
Matsuzaki, Y., Katsushima, N., Nagai, Y., Shoji, M., Itagaki, T., Sakamoto, M., . . . & Nishimura, H. (2006). Clinical features of influenza C virus infection in children. The Journal of infectious diseases, 193(9), 1229-1235.
Maverakis, E., Kim, K., Shimoda, M., Gershwin, M. E., Wilken, R., Raychaudhuri, S., . . . & Lebrilla, C. B. (2015). Glycans in the immune system and The Altered Glycan Theory of Autoimmunity: a critical review. Journal of autoimmunity, 57, 1-13.
McGeoch, D., Fellner, P., & Newton, C. (1976). Influenza virus genome consists of eight distinct RNA species. Proceedings of the National Academy of Sciences, 73(9), 3045-3049.
Mechtcheriakova, I. A., Eldarov, M. A., Nicholson, L., Shanks, M., Skryabin, K. G., & Lomonossoff, G. P. (2006). The use of viral vectors to produce hepatitis B virus core particles in plants. Journal of virological methods, 131(1), 10-15.
Milich, D. R., & McLachlan, A. (1986). The nucleocapsid of hepatitis B virus is both a T-cell-independent and a T-cell-dependent antigen. Science, 234(4782), 1398-1401.
Milich, D. R., Peterson, D. L., Schodel, F., Jones, J. E., & Hughes, J. L. (1995). Preferential recognition of hepatitis B nucleocapsid antigens by Th1 or Th2 cells is epitope and major histocompatibility complex dependent. Journal of virology, 69(5), 2776-2785.
Mitnaul, L. J., Matrosovich, M. N., Castrucci, M. R., Tuzikov, A. B., Bovin, N. V., Kobasa, D., & Kawaoka, Y. (2000). Balanced hemagglutinin and neuraminidase activities are critical for efficient replication of influenza A virus. Journal of virology, 74(13), 6015-6020.
Mosmann, T. R., & Coffman, R. L. (1989). TH1 and TH2 cells: different patterns of lymphokine secretion lead to different functional properties. Annual review of immunology, 7(1), 145-173.
Mosnier, A., Daviaud, I., Casalegno, J. S., Ruetsch, M., Burugorri, C., Nauleau, E., . . . & Cohen, J. M. (2017). Influenza B burden during seasonal influenza epidemics in France. Medecine et maladies infectieuses, 47(1), 11-17.
Most, J., & Weiss, G. (2016). Consecutive infections with influenza A and B virus in children during the 2014-2015 seasonal influenza epidemic. The Journal of infectious diseases, 214(8), 1139-1141.
Nair, H., Brooks, W. A., Katz, M., Roca, A., Berkley, J. A., Madhi, S. A., . . . & Krishnan, A. (2011). Global burden of respiratory infections due to seasonal influenza in young children: a systematic review and meta-analysis. The Lancet, 378(9807), 1917-1930.
Nandi, S., Kwong, A. T., Holtz, B. R., Erwin, R. L., Marcel, S., and McDonald, K. A. (2016). Techno-economic analysis of a transient plant-based platform for monoclonal antibody production. MAbs 8, 1456-1466. doi:10.1080/19420862.2016.1227901.
Neirynck, S., Deroo, T., Saelens, X., Vanlandschoot, P., Jou, W. M., & Fiers, W. (1999). A universal influenza A vaccine based on the extracellular domain of the M2 protein. Nature medicine, 5(10), 1157-1163.
Nemchinov, L. G., & Natilla, A. (2007). Transient expression of the ectodomain of matrix protein 2 (M2e) of avian influenza A virus in plants. Protein expression and purification, 56(2), 153-159.
Neuberger, M. S., and Rajewsky, K. (1981). Activation of mouse complement by monoclonal mouse antibodies. Eur. J. Immunol. 11, 1012-1016. doi:10.1002/eji.1830111212.
Niwa, R., Natsume, A., Uehara, A., Wakitani, M., Iida, S., Uchida, K., . . . & Shitara, K. (2005). IgG subclass-independent improvement of antibody-dependent cellular cytotoxicity by fucose removal from Asn297-linked oligosaccharides. Journal of immunological methods, 306(1-2), 151-160.
Nobusawa, E., & Sato, K. (2006). Comparison of the mutation rates of human influenza A and B viruses. Journal of virology, 80(7), 3675-3678.
Oliveira, E. R. A., Mohana-Borges, R., de Alencastro, R. B., and Horta, B. A. C. (2017). The flavivirus capsid protein: Structure, function and perspectives towards drug design. Virus Res. 227, 115-123.
Paprotka, T., Deuschle, K., Pilartz, M., & Jeske, H. (2015). Form follows function in geminiviral minichromosome architecture. Virus research, 196, 44-55.
Pastrana, D. V., Gambhira, R., Buck, C. B., Pang, Y. Y. S., Thompson, C. D., Culp, T. D., et al. (2005). Cross-neutralization of cutaneous and mucosal Papillomavirus types with anti-sera to the amino terminus of L2. Virology 337, 365-372. doi:10.1016/j.virol.2005.04.011.
Paules, C. I., Sullivan, S. G., Subbarao, K., & Fauci, A. S. (2018). Chasing seasonal influenza—The need for a universal influenza vaccine. New England Journal of Medicine, 378(1), 7-9.
Pepponi, I., Diogo, G. R., Stylianou, E., van Dolleweerd, C. J., Drake, P. M. W., Paul, M. J., et al. (2014). Plant-derived recombinant immune complexes as self-adjuvanting TB immunogens for mucosal boosting of BCG. Plant Biotechnol. J. 12, 840-850. doi:10.1111/pbi.12185.
Peyret, H. (2015). A protocol for the gentle purification of virus-like particles produced in plants. Journal of virological methods, 225, 59-63.
Peyret, H., Gehin, A., Thuenemann, E. C., Blond, D., El Turabi, A., Beales, L., et al. (2015). Tandem fusion of hepatitis B core antigen allows assembly of virus-like particles in bacteria and plants with enhanced capacity to accommodate foreign proteins. PLoS One 10. doi:10.1371/journal.pone.0120751.
Phoolcharoen, W., Bhoo, S. H., Lai, H., Ma, J., Arntzen, C. J., Chen, Q., & Mason, H. S. (2011). Expression of an immunogenic Ebola immune complex in Nicotiana benthamiana. Plant biotechnology journal, 9(7), 807-816.
Phoolcharoen, W., Dye, J. M., Kilbourne, J., Piensook, K., Pratt, W. D., Arntzen, C. J., et al. (2011). A nonreplicating subunit vaccine protects mice against lethal Ebola virus challenge. Proc. Natl. Acad. Sci. U.S.A 108, 20695-700. doi:10.1073/pnas.1117715108.
Pumpens, P., & Grens, E. (2001). HBV core particles as a carrier for B cell/T cell epitopes. Intervirology, 44(2-3), 98-114.
Pushko, P., Tretyakova, I., Hidajat, R., Zsak, A., Chrzastek, K., Tumpey, T. M., & Kapczynski, D. R. (2017). Virus-like particles displaying H5, H7, H9 hemagglutinins and N1 neuraminidase elicit protective immunity to heterologous avian influenza viruses in chickens. Virology, 501, 176-182.
Putri, W. C., Muscatello, D. J., Stockwell, M. S., & Newall, A. T. (2018). Economic burden of seasonal influenza in the United States. Vaccine, 36(27), 3960-3966.
Rabaan, A. A., Bazzi, A. M., Al-Ahmed, S. H., Al-Ghaith, M. H., and Al-Tawfiq, J. A. (2017). Overview of Zika infection, epidemiology, transmission and control measures. J. Infect. Public Health 10, 141-149.
Radaev, S. (2002). Recognition of immunoglobulins by Fc7 receptors. Mol. Immunol. 38, 1073-1083. doi:10.1016/S0161-5890(02)00036-6.
Ramirez, A., Morris, S., Maucourant, S., D'Ascanio, I., Crescente, V., Lu, I. N., . . . & Rosenberg, W. (2018). A virus-like particle vaccine candidate for influenza A virus based on multiple conserved antigens presented on hepatitis B tandem core particles. Vaccine, 36(6), 873-880.
Rohovie, M. J., Nagasawa, M., & Swartz, J. R. (2017). Virus-like particles: Next-generation nanoparticles for targeted therapeutic delivery. Bioengineering & translational medicine, 2(1), 43-57.
Rolfes, M. A., Foppa, I. M., Garg, S., Flannery, B., Brammer, L., Singleton, J. A., . . . & Reed, C. (2018). Annual estimates of the burden of seasonal influenza in the United States: a tool for strengthening influenza surveillance and preparedness. Influenza and other respiratory viruses, 12(1), 132-137.
Rosenthal, S. H., Diamos, A. G., and Mason, H. S. (2018) An intronless form of the tobacco extensin gene terminator strongly enhances transient gene expression in plant leaves. Plant Mol Biol. 2018 Feb. 10. doi: 10.1007/s11103-018-0708-y. [Epub ahead of print].
Rybicki, E. P. (2010). Plant-made vaccines for humans and animals. Plant biotechnology journal, 8(5), 620-637.
Santi, L., Batchelor, L., Huang, Z., Hjelm, B., Kilbourne, J., Arntzen, C. J., Chen, Q., and Mason, H. S. (2008). An efficient plant viral expression system generating orally immunogenic Norwalk virus-like particles. Vaccine 26, 1846-1854.
Schellenbacher, C., Roden, R. B. S., and Kirnbauer, R. (2017). Developments in L2-based human papillomavirus (HPV) vaccines. Virus Res. 231, 166-175. doi:10.1016/j.virusres.2016.11.020.
Schödel, F., Moriarty, A. M., Peterson, D. L., Zheng, J. A., Hughes, J. L., Will, H., . . . & Milich, D. R. (1992). The position of heterologous epitopes inserted in hepatitis B virus core particles determines their immunogenicity. Journal of virology, 66(1), 106-114.
Scorza, F. B., Tsvetnitsky, V., & Donnelly, J. J. (2016). Universal influenza vaccines: Shifting to better vaccines. Vaccine, 34(26), 2926-2933.
Sharma, D. P., Ramsay, A. J., Maguire, D. J., Rolph, M. S., & Ramshaw, I. A. (1996). Interleukin-4 mediates down regulation of antiviral cytokine expression and cytotoxic T-lymphocyte responses and exacerbates vaccinia virus infection in vivo. Journal of Virology, 70(10), 7103-7107.
Shields, R. L., Lai, J., Keck, R., O'Connell, L. Y., Hong, K., Meng, Y. G., . . . & Presta, L. G. (2002). Lack of fucose on human IgG1 N-linked oligosaccharide improves binding to human FcγRIII and antibody-dependent cellular toxicity. Journal of Biological Chemistry, 277(30), 26733-26740.
Simón, D., Fajardo, A., Sóñora, M., Delfraro, A., and Musto, H. (2017). Host influence in the genomic composition of flaviviruses: A multivariate approach. Biochem. Biophys. Res. Commun. 492, 572-578.
Skehel, J. J., & Wiley, D. C. (2000). Receptor binding and membrane fusion in virus entry: the influenza hemagglutinin. Annual review of biochemistry, 69(1), 531-569.
Skowronski, D. M., Chambers, C., De Serres, G., Dickinson, J. A., Winter, A. L., Hickman, R., . . . & Gubbay, J. B. (2018). Early season co-circulation of influenza A (H3N2) and B (Yamagata): interim estimates of 2017/18 vaccine effectiveness, Canada, January 2018. Eurosurveillance, 23(5).
Smith, D. B., Gaunt, E. R., Digard, P., Templeton, K., & Simmonds, P. (2016). Detection of influenza C virus but not influenza D virus in Scottish respiratory samples. Journal of Clinical Virology, 74, 50-53.
Stanley, J. (1993). Geminiviruses: plant viral vectors. Current opinion in genetics & development, 3(1), 91-96.
Stemmer, W. P., Crameri, A., Ha, K. D., Brennan, T. M., & Heyneker, H. L. (1995). Single-step assembly of a gene and entire plasmid from large numbers of oligodeoxyribonucleotides. Gene, 164, 49-53.
Stepanova, L. A., Mardanova, E. S., Shuklina, M. A., Blokhina, E. A., Kotlyarov, R. Y., Potapchuk, M. V., . . . & Ravin, N. V. (2018). Flagellin-fused protein targeting M2e and HA2 induces potent humoral and T-cell responses and protects mice against various influenza viruses a subtypes. Journal of biomedical science, 25(1), 33.
Stettler, K., Beltramello, M., Espinosa, D. A., Graham, V., Cassotta, A., Bianchi, S., Vanzetta, F., Minola, A., Jaconi, S., Mele, F., et al. (2016). Specificity, cross-reactivity, and function of antibodies elicited by Zika virus infection. Science (80-.). 353, 823-826.
Strasser, R., Stadlmann, J., Schahs, M., Stiegler, G., Quendler, H., Mach, L., et al. (2008). Generation of glyco-engineered Nicotiana benthamiana for the production of monoclonal antibodies with a homogeneous human-like N-glycan structure. Plant Biotechnol. J. 6, 392-402. doi:10.1111/j.1467-7652.2008.00330.x.
Streatfield, S. J., Jilka, J. M., Hood, E. E., Turner, D. D., Bailey, M. R., Mayor, J. M., . . . & Tizard, I. R. (2001). Plant-based vaccines: unique advantages. Vaccine, 19(17), 2742-2748.
Su, S., Fu, X., Li, G., Kerlin, F., & Veit, M. (2017). Novel Influenza D virus: Epidemiology, pathology, evolution and biological characteristics. Virulence, 8(8), 1580-1591.
Suarez, D. L. (2016). Influenza A virus. Animal Influenza, 1-30.
Sullivan, S. G., Chilver, M. B., Carville, K. S., Deng, Y. M., Grant, K. A., Higgins, G., . . . & Tran, T. (2017). Low interim influenza vaccine effectiveness, Australia, 1 May to 24 Sep. 2017. Eurosurveillance, 22(43).
Takai, T., Li, M., Sylvestre, D., Clynes, R., and Ravetch, J. V (1994). FcR 7 chain deletion results in pleiotrophic effector cell defects. Cell 76, 519-529. doi:10.1016/0092-8674(94)90115-5.
Taylor, A., Foo, S.-S., Bruzzone, R., Vu Dinh, L., King, N. J. C., and Mahalingam, S. (2015). Fc receptors in antibody-dependent enhancement of viral infections. Immunol. Rev. 268, 340-364.
Thompson, W. W., Shay, D. K., Weintraub, E., Brammer, L., Bridges, C. B., Cox, N. J., & Fukuda, K. (2004). Influenza-associated hospitalizations in the United States. Jama, 292(11), 1333-1340.
Tiwari, S., Verma, P. C., Singh, P. K., & Tuli, R. (2009). Plants as bioreactors for the production of vaccine antigens. Biotechnology advances, 27(4), 449-467.
Turley, C. B., Rupp, R. E., Johnson, C., Taylor, D. N., Wolfson, J., Tussey, L., . . . & Shaw, A. (2011). Safety and immunogenicity of a recombinant M2e-flagellin influenza vaccine (STF2. 4×M2e) in healthy adults. Vaccine, 29(32), 5145-515
Tusé, D., Tu, T., and McDonald, K. A. (2014). Manufacturing Economics of Plant-Made Biologics: Case Studies in Therapeutic and Industrial Enzymes. Biomed Res. Int. 2014, 1-16. doi:10.1155/2014/256135.
Tusé, D., Tu, T., and McDonald, K. A. (2014). Manufacturing Economics of Plant-Made Biologics: Case Studies in Therapeutic and Industrial Enzymes. Biomed Res. Int. 2014, 1-16.
Van den Hoecke, S., Ehrhardt, K., Kolpe, A., El Bakkouri, K., Deng, L., Grootaert, H., . . . & Schotsaert, M. (2017). Hierarchical and redundant roles of activating FcγRs in protection against influenza disease by M2e-specific IgG1 and IgG2a antibodies. Journal of virology, 91(7), e02500-16.
Vesikari, T., Brodszki, N., Van Damme, P., Diez-Domingo, J., Icardi, G., Petersen, L. K., et al. (2015). A Randomized, Double-Blind, Phase III Study of the Immunogenicity and Safety of a 9-Valent Human Papillomavirus L Virus-Like Particle Vaccine (V503) Versus Gardasil® in 9-15-Year-Old Girls. Pediatr. Infect. Dis. J. 34, 992-998. doi:10.1097/INF.0000000000000773.
Webster, R. G., Laver, W. G., Air, G. M., & Schild, G. C. (1982). Molecular mechanisms of variation in influenza viruses. Nature, 296(5853), 115-121.
Wen Y-M, Mu L, Shi Y. Immunoregulatory functions of immune complexes in vaccine and therapy. EMBO Mol Med 2016; 8:1120-33. doi:10.15252/emmm.201606593.
Wheeler, C. M., Kjaer, S. K., Sigurdsson, K., Iversen, O., Hernandez-Avila, M., Perez, G., et al. (2009). The Impact of Quadrivalent Human Papillomavirus (HPV; Types 6, 11, 16, and 18) L1 Virus-Like Particle Vaccine on Infection and Disease Due to Oncogenic Nonvaccine HPV Types in Sexually Active Women Aged 16-26 Years. J. Infect. Dis. 199, 936-944. doi:10.1086/597309.
Whitacre, D. C., Lee, B. O., & Milich, D. R. (2009). Use of hepadnavirus core proteins as vaccine platforms. Expert Review of Vaccines, 8(11), 1565-1573.
Whitacre, D. C., Lee, B. O., and Milich, D. R. (2009). Use of hepadnavirus core proteins as vaccine platforms. Expert Rev. Vaccines 8, 1565-1573. doi:10.1586/erv.09.121.
Wilder-Smith, A., Vannice, K., Durbin, A., Hombach, J., Thomas, S. J., Thevarjan, I., and Simmons, C. P. (2018). Zika vaccines and therapeutics: landscape analysis and challenges ahead. BMC Med. 16.
Wilson, J. A., Hevey, M., Bakken, R., Guest, S., Bray, M., Schmaljohn, A. L. and Hart, M. K. (2000) Epitopes involved in antibody-mediated protection from Ebola virus. Science, 287, 1664-1666.
World Health Organization. (2018). Influenza (Seasonal) Fact Sheet. Retrieved Feb. 12, 2019.
Yang, M., Dent, M., Lai, H., Sun, H., and Chen, Q. (2017). Immunization of Zika virus envelope protein domain III induces specific and neutralizing immune responses against Zika virus. Vaccine 35, 4287-4294.
Yang, M., Lai, H., Sun, H., & Chen, Q. (2017). Virus-like particles that display Zika virus envelope protein domain III induce potent neutralizing immune responses in mice. Scientific reports, 7(1), 7679.
Yang, M., Sun, H., Lai, H., Hurtado, J., and Chen, Q. (2018). Plant-produced Zika virus envelope protein elicits neutralizing immune responses that correlate with protective immunity against Zika virus in mice. Plant Biotechnol. J. 16, 572-580.
Zebedee, S. L., & Lamb, R. A. (1988). Influenza A virus M2 protein: monoclonal antibody restriction of virus growth and detection of M2 in virions. Journal of virology, 62(8), 2762-2772.
Zeitlin, L., Pettitt, J., Scully, C., Bohorova, N., Kim, D., Pauly, M., et al. (2011). Enhanced potency of a fucose-free monoclonal antibody being developed as an Ebola virus immunoprotectant. Proc. Natl. Acad. Sci. 108, 20690-20694. doi:10.1073/pnas.1108360108.
Zhang, J., Fan, H. Y., Zhang, Z., Zhang, J., Zhang, J., Huang, J. N., . . . & Liao, M. (2016). Recombinant baculovirus vaccine containing multiple M2e and adjuvant LTB induces T cell dependent, cross-clade protection against H5N1 influenza virus in mice. Vaccine, 34(5), 622-629.
Zhang, X., Jia, R., Shen, H., Wang, M., Yin, Z., and Cheng, A. (2017). Structures and functions of the envelope glycoprotein in flavivirus infections. Viruses 9.
Zhou, C., Zhou, L., & Chen, Y. H. (2012). Immunization with high epitope density of M2e derived from 2009 pandemic H1N1 elicits protective immunity in mice. Vaccine, 30(23), 3463-3469.

1. A recombinant immune complex comprising:
an immunoglobulin heavy chain;
an epitope tag, wherein the immunoglobulin heavy chain binds the epitope tag; and
a fragment of the virus protein,
wherein the fragment of the virus protein is linked to the C-terminus of the immunoglobulin heavy chain, the epitope tag is linked to the C-terminus of the fragment of the virus protein, and the epitope tag is from a different protein than the virus protein.

2. The recombinant immune complex of claim 1, wherein the epitope tag is an Ebola antigen.

3. The recombinant immune complex of claim 1, wherein the immunoglobulin heavy chain is the immunoglobulin heavy chain of humanized 6D8 monoclonal antibody and the epitope tag is the 6D8 epitope tag.

4. The recombinant immune complex of claim 1, further comprising an immunoglobulin light chain.

5. The recombinant immune complex of claim 1, wherein the virus protein is HPV minor capsid protein L2 (GenBank Accession No. AGH32604.1).

6. The recombinant immune complex of claim 5, wherein the fragment of the virus protein comprises at least 8 continuous amino acids from the first 200 amino acid residues from the N-terminus of HPV minor capsid protein L2.

7. The recombinant immune complex of claim 5, wherein the fragment of the virus protein comprises at least one peptide sequence from the HPV minor capsid protein L2 selected from the group consisting of: amino acid residues 17-36, amino acid residues 56-75, amino acid residues 65-85, and amino acid residues 96-115.

8. The recombinant immune complex of claim 1, wherein the virus protein is M2e.

9. The recombinant immune complex of claim 8, wherein the fragment of the virus protein comprises the amino acid sequence set forth in SEQ ID NO. 9.

10. The recombinant immune complex of claim 8, wherein the fragment of the virus protein comprises the amino acid sequence set forth in SEQ ID NO. 10.

11. The recombinant immune complex of claim 1, wherein the virus protein is selected from the group consisting of: zika virus E protein domain 3 protein (ZE3), zika virus fusion loop antigen (ZE), and zika virus soluble envelope protein (Zse).

12. The recombinant immune complex of claim 11, wherein the fragment of the virus protein comprises amino acid residues 591-696 or amino acid residues 352-412 of GenBank Accession No. AMC13911.1.

13. The recombinant immune complex of claim 11, wherein the recombinant immune complex is encoded by the plasmid of SEQ ID NO. 36 or SEQ ID NO. 37.

14. A method of generating an immune response against a virus in a mammalian subject, the method comprising administering to the mammalian subject a recombinant immune complex (RIC), the RIC comprising:
an immunoglobulin heavy chain;
an epitope tag, wherein the immunoglobulin heavy chain binds the epitope tag; and
a fragment of the virus protein,
wherein the fragment of the virus protein is linked to the C-terminus of the immunoglobulin heavy chain, the epitope tag is linked to the C-terminus of the fragment of the virus protein, and the epitope tag is from a different protein than the virus protein.

15. The method of claim 14, wherein the epitope tag is an Ebola antigen.

16. The method of claim 14, wherein the immunoglobulin heavy chain is the immunoglobulin heavy chain of humanized 6D8 monoclonal antibody and the epitope tag is the 6D8 epitope tag.

17. The method of claim 14, wherein the RIC further comprises an immunoglobulin light chain.


SEQUENCE LISTING (45 sequences)

SEQ ID NO: 1 - DNA, 30 bp, synthetic construct
  misc_feature 1..30: note = primer
  source 1..30: mol_type = other DNA
cgtctagagtccgcaacccaactttacaag

SEQ ID NO: 2 - DNA, 23 bp, synthetic construct
  misc_feature 1..23: note = primer
  source 1..23: mol_type = other DNA
ggactagttggggcaccagcatc

SEQ ID NO: 3 - DNA, 28 bp, synthetic construct
  misc_feature 1..28: note = primer
  source 1..28: mol_type = other DNA
caggatccgcaacccaactttacaagac

SEQ ID NO: 4 - protein, 109 aa, Human papillomavirus
  source 1..109: note = Type 16
SATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTS

SEQ ID NO: 5 - protein, 20 aa, Human papillomavirus
  source 1..20: note = Type 16
QLYKTCKQAGTCPPDIIPKV

SEQ ID NO: 6 - protein, 20 aa, Human papillomavirus
  source 1..20: note = Type 16
LQYGSMGVFFGGLGIGTGSG

SEQ ID NO: 7 - protein, 21 aa, Human papillomavirus
  source 1..21: note = Type 16
FGGLGIGTGSGTGGRTGYIPL

SEQ ID NO: 8 - protein, 20 aa, Human papillomavirus
  source 1..20: note = Type 16
DPVGPSDPSIVSLVEETSFI

SEQ ID NO: 9 - protein, 23 aa, Influenza virus
  source 1..23: note = Type A
SLLTEVETPIRNEWGCRCNDSSD

SEQ ID NO: 10 - protein, 51 aa, Influenza virus
  source 1..51: note = Type A
SLLTEVETPIRNEWGCRCNDSSDGGSGGSLLTEVETPIRNEWGCRCNDSSD

SEQ ID NO: 11 - DNA, 210 bp, synthetic construct
  misc_feature 1..210: note = primer
  source 1..210: mol_type = other DNA
gtaaaacgacggccagtggatcctctttgcttaccgaggttgagacccctattagaaacgagtggggttgcagatgtaacgattcttccgacggaggttctggaggttcccttttgactgaagtggagactccaatcaggaacgaatggggatgcagatgcaacgactcctctgacggaggtggaactagtcatggtcatagctgtttcc

SEQ ID NO: 12 - DNA, 27 bp, synthetic construct
  misc_feature 1..27: note = primer
  source 1..27: mol_type = other DNA
tagccatgggatcctctttgcttaccg

SEQ ID NO: 13 - DNA, 27 bp, synthetic construct
  misc_feature 1..27: note = primer
  source 1..27: mol_type = other DNA
tcgctcgagactagttccacctccgtc

SEQ ID NO: 14 - DNA, 18 bp, synthetic construct
  misc_feature 1..18: note = primer
  source 1..18: mol_type = other DNA
tgaggctcttcacaatca

SEQ ID NO: 15 - DNA, 26 bp, synthetic construct
  misc_feature 1..26: note = primer
  source 1..26: mol_type = other DNA
cttcttcttcttcttttctcattgtc

SEQ ID NO: 16 - DNA, 22 bp, synthetic construct
  misc_feature 1..22: note = primer
  source 1..22: mol_type = other DNA
caatttgctttgcattcttgac

SEQ ID NO: 17 - DNA, 17 bp, synthetic construct
  misc_feature 1..17: note = primer
  source 1..17: mol_type = other DNA
gtaaaacgacggccagt

SEQ ID NO: 18 - DNA, 19 bp, synthetic construct
  misc_feature 1..19: note = primer
  source 1..19: mol_type = other DNA
ggaaacagctatgaccatg

SEQ ID NO: 19 - DNA, 14158 bp, synthetic construct
  misc_feature 1..14158: note = pBYR2eK2M-HBcheM2e
  source 1..14158: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacaccatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagaggatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttggaggttctggtggatcaggaggttccggtggttctggaggttccggaatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagagggtaccggtggaggcggttcaggcggaggtggatcctctttgcttaccgaggttgagacccctattagaaacgagtggggttgcagatgtaacgattcttccgacggaggttctggaggttcccttttgactgaagtggagactccaatcaggaacgaatggggatgcagatgcaacgactcctctgacggaggtggaactagtggaggttctggaggatctggttctagtggaggttctggtggagatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttcgaagaagggacaggggcagatcccctagacgtagaactcccagccctagaagaaggagatccccatctcctaggcgtagataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 20 - DNA, 18342 bp, synthetic construct
  misc_feature 1..18342: note = pBYR11eMa-h6D8M2e
  source 1..18342: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggatggtcttgcatcatactctttcttgttgcaactgctacaggtgtccactctgatgttcagcttctcgagtctggaggtggtcttgtgcaacctggaggttccttgagactctcctgtgcagcttcagggtttgacttcagtaggtactggatgagttgggttcgtcaagctcctgggaaaggactagaatggattggagagatcaatccagattcaagtaccatcaactatactccatctctgaaggatcgcttcaccatttccagagacaatgccaagaacacgttgtatcttcagatgaacagcttgaggactgaagacacagccttgtactactgcacaagacagggctatggctacaactactggggtcaaggcaccactgtcacagtgtcttcagctagcaccaaaggtccatcggtctttccactggcaccttcttccaagagtacttctggaggcacagctgcactgggttgtcttgtcaaggactactttccagaacctgttacggtttcgtggaactcaggtgctctgaccagtggagtgcacacctttccagctgttcttcagtcctcaggattgtattctcttagcagtgttgtgactgttccatcctcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtctccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaaggaggtggcggatcaggtggaggcggttcaggcggaggtggatcctctttgcttaccgaggttgagacccctattagaaacgagtggggttgcagatgtaacgattcttccgacggaggttctggaggttcccttttgactgaagtggagactccaatcaggaacgaatggggatgcagatgcaacgactcctctgacggaggtggaactagtcataacactcctgtttacaagctggacatatctgaggcaactcaataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatcggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatgggatggtcttgcatcattctcttcttggtagccacagctacaggtgtccactccgatgttttgatgactcaaagccctctctcacttcctgtgactcttggacagcccgcatccatatcttgcagatctagtcagagtattgttcatagtaacggcaacacctacttggaatggtatctgcagaaaccaggccagtctccaaagcttctgatctacaaggcttccaatcgtttctctggtgtcccagacaggtttagtggcagtggatcagggactgacttcacattgaagatcagcagagttgaggctgaagatgcgggagtgtactattgtcttcaaggttcacatgttccgtcaacgtttggaggtgggaccaaagtggagatcaagactgttgcggcgccatctgtcttcatctttcctccatctgatgaacaactcaagtctggaactgcttctgttgtgtgccttctgaacaacttctatcctagagaagccaaagtacagtggaaggttgacaatgctcttcaatcaggtaactcccaggagagtgtcacagagcaagattccaaggattccacctacagcctctcaagtaccttgacgttgagcaaggcagactatgagaaacacaaagtgtacgcatgcgaagtcactcatcagggcctgtcatcacccgtgacaaagagcttcaacaggggagagtgttaggtaccgagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 21 - DNA, 18 bp, synthetic construct
  misc_feature 1..18: note = primer
  source 1..18: mol_type = other DNA
aatcccactatccttcgc

SEQ ID NO: 22 - DNA, 27 bp, synthetic construct
  misc_feature 1..27: note = primer
  source 1..27: mol_type = other DNA
gcggtctccaccagaagcaagagaagc

SEQ ID NO: 23 - DNA, 34 bp, synthetic construct
  misc_feature 1..34: note = primer
  source 1..34: mol_type = other DNA
gtcggatccgatgttcagcttcttgagtctggag

SEQ ID NO: 24 - DNA, 32 bp, synthetic construct
  misc_feature 1..32: note = primer
  source 1..32: mol_type = other DNA
gcgagctcttatctacgcctaggagatgggga

SEQ ID NO: 25 - DNA, 32 bp, synthetic construct
  misc_feature 1..32: note = primer
  source 1..32: mol_type = other DNA
gcggtctcgtggtatggacattgacccttaca

SEQ ID NO: 26 - DNA, 23 bp, synthetic construct
  misc_feature 1..23: note = primer
  source 1..23: mol_type = other DNA
aagcttgttgttgtgactccgag

SEQ ID NO: 27 - DNA, 42 bp, synthetic construct
  misc_feature 1..42: note = primer
  source 1..42: mol_type = other DNA
ctagtggtggatcaggaggttctggtggttctggaggttcag

SEQ ID NO: 28 - DNA, 42 bp, synthetic construct
  misc_feature 1..42: note = primer
  source 1..42: mol_type = other DNA
gatcctgaacctccagaaccaccagaacctcctgatccacca

SEQ ID NO: 29 - DNA, 27 bp, synthetic construct
  misc_feature 1..27: note = primer
  source 1..27: mol_type = other DNA
gcgggatccaagggcgtgtcatactcc

SEQ ID NO: 30 - DNA, 30 bp, synthetic construct
  misc_feature 1..30: note = primer
  source 1..30: mol_type = other DNA
ggggtctcgtggtaagggcgtgtcatactc

SEQ ID NO: 31 - DNA, 24 bp, synthetic construct
  misc_feature 1..24: note = primer
  source 1..24: mol_type = other DNA
ccgactagtgctaccactcctgtg

SEQ ID NO: 32 - DNA, 30 bp, synthetic construct
  misc_feature 1..30: note = primer
  source 1..30: mol_type = other DNA
gagggatccgaggcttcaatttcagacatg

SEQ ID NO: 33 - DNA, 27 bp, synthetic construct
  misc_feature 1..27: note = primer
  source 1..27: mol_type = other DNA
gggactagtggagcaagcgaatttagc

SEQ ID NO: 34 - DNA, 14339 bp, synthetic construct
  misc_feature 1..14339: note = pBYe3R2K2Mc-BAHBcheZE3
  source 1..14339: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagaggatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttggaggttctggtggatcaggaggttccggtggttctggaggttccggaatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagagggtaccggtggaggcggttcaggcggaggtggatccaagggcgtgtcatactccttgtgtaccgctgccttcacattcaccaagatcccggctgaaacactccacggaaccgttaccgtggaggtccaatacgccggtacagatggaccttgcaaggttccagctcagatggcggtggacatgcaaactcttaccccagttggaaggttgattaccgctaaccccgttatcactgaaagcactgagaactctaagatgatgttggaacttgatccaccattcggtgactcttacattgtcattggtgtgggagagaagaagatcacccaccactggcacaggagtggtagcactagtggaggttctggaggatctggttctagtggaggttctggtggagatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttcgaagaagggacaggggcagatcccctagacgtagaactcccagccctagaagaaggagatccccatctcctaggcgtagataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccctaaaaccaaaatccagtgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 35 - DNA, 14162 bp, synthetic construct
  misc_feature 1..14162: note = pBYe3R2K2Mc-BAHBcheZE62-122
  source 1..14162: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagaggatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttggaggttctggtggatcaggaggttccggtggttctggaggttccggaatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatcttgagggaggttcaggtggatccgaggcttcaatttcagacatggctagtgacagccgttgcccaacacaaggtgaagcctaccttgacaagcaatcagacactcaatatgtgtgcaagagaacattggtggacagaggttggggaaacggatgtggacttttcggtaagggaagcctcgtgacatgcgctaaattcgcttgctccactagtggaggttctggtggagatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttcgaagaagggacaggggcagatcccctagacgtagaactcccagccctagaagaaggagatccccatctcctaggcgtagataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccctaaaaccaaaatccagtgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 36 - DNA, 17977 bp, synthetic construct
  misc_feature 1..17977: note = pBYR11eM-h6D8-ZEFL62
  source 1..17977: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggatggtcttgcatcatactctttcttgttgcaactgctacaggtgtccactctgatgttcagcttctcgagtctggaggtggtcttgtgcaacctggaggttccttgagactctcctgtgcagcttcagggtttgacttcagtaggtactggatgagttgggttcgtcaagctcctgggaaaggactagaatggattggagagatcaatccagattcaagtaccatcaactatactccatctctgaaggatcgcttcaccatttccagagacaatgccaagaacacgttgtatcttcagatgaacagcttgaggactgaagacacagccttgtactactgcacaagacagggctatggctacaactactggggtcaaggcaccactgtcacagtgtcttcagctagcaccaaaggtccatcggtctttccactggcaccttcttccaagagtacttctggaggcacagctgcactgggttgtcttgtcaaggactactttccagaacctgttacggtttcgtggaactcaggtgctctgaccagtggagtgcacacctttccagctgttcttcagtcctcaggattgtattctcttagcagtgttgtgactgttccatcctcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtctccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaaggaggtggcggatcaggtggaggcggttcaggcggaggtggatccaagggcgtgtcatactccttgtgtaccgctgccttcacattcaccaagatcccggctgaaacactccacggaaccgttaccgtggaggtccaatacgccggtacagatggaccttgcaaggttccagctcagatggcggtggacatgcaaactcttaccccagttggaaggttgattaccgctaaccccgttatcactgaaagcactgagaactctaagatgatgttggaacttgatccaccattcggtgactcttacattgtcattggtgtgggagagaagaagatcacccaccactggcacaggagtggtagcactagtcataacactcctgtttacaagctggacatatctgaggcaactcaataagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgagaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatcggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatgggatggtcttgcatcattctcttcttggtagccacagctacaggtgtccactccgatgttttgatgactcaaagccctctctcacttcctgtgactcttggacagcccgcatccatatcttgcagatctagtcagagtattgttcatagtaacggcaacacctacttggaatggtatctgcagaaaccaggccagtctccaaagcttctgatctacaaggcttccaatcgtttctctggtgtcccagacaggtttagtggcagtggatcagggactgacttcacattgaagatcagcagagttgaggctgaagatgcgggagtgtactattgtcttcaaggttcacatgttccgtcaacgtttggaggtgggaccaaagtggagatcaagactgttgcggcgccatctgtcttcatctttcctccatctgatgaacaactcaagtctggaactgcttctgttgtgtgccttctgaacaacttctatcctagagaagccaaagtacagtggaaggttgacaatgctcttcaatcaggtaactcccaggagagtgtcacagagcaagattccaaggattccacctacagcctctcaagtaccttgacgttgagcaaggcagactatgagaaacacaaagtgtacgcatgcgaagtcactcatcagggcctgtcatcacccgtgacaaagagcttcaacaggggagagtgttaggtaccgagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 37 - DNA, 17931 bp, synthetic construct
  misc_feature 1..17931: note = pBYR11eM-h6D8ZE3
  source 1..17931: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggatggtcttgcatcatactctttcttgttgcaactgctacaggtgtccactctgatgttcagcttctcgagtctggaggtggtcttgtgcaacctggaggttccttgagactctcctgtgcagcttcagggtttgacttcagtaggtactggatgagttgggttcgtcaagctcctgggaaaggactagaatggattggagagatcaatccagattcaagtaccatcaactatactccatctctgaaggatcgcttcaccatttccagagacaatgccaagaacacgttgtatcttcagatgaacagcttgaggactgaagacacagccttgtactactgcacaagacagggctatggctacaactactggggtcaaggcaccactgtcacagtgtcttcagctagcaccaaaggtccatcggtctttccactggcaccttcttccaagagtacttctggaggcacagctgcactgggttgtcttgtcaaggactactttccagaacctgttacggtttcgtggaactcaggtgctctgaccagtggagtgcacacctttccagctgttcttcagtcctcaggattgtattctcttagcagtgttgtgactgttccatcctcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtctccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaaggaggtggcggatcaggtggaggcggttcaggcggaggtggatccgaggcttcaatttcagacatggctagtgacagccgttgcccaacacaaggtgaagcctaccttgacaagcaatcagacactcaatatgtgtgcaagagaacattggtggacagaggttggggaaacggatgtggacttttcggtaagggaagcctcgtgacatgcgctaaattcgcttgctccactagtcataacactcctgtttacaagctggacatatctgaggcaactcaataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatcggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggatggtcttgcatcattctcttcttggtagccacagctacaggtgtccactccgatgttttgatgactcaaagccctctctcacttcctgtgactcttggacagcccgcatccatatcttgcagatctagtcagagtattgttcatagtaacggcaacacctacttggaatggtatctgcagaaaccaggccagtctccaaagcttctgatctacaaggcttccaatcgtttctctggtgtcccagacaggtttagtggcagtggatcagggactgacttcacattgaagatcagcagagttgaggctgaagatgcgggagtgtactattgtcttcaaggttcacatgttccgtcaacgtttggaggtgggaccaaagtggagatcaagactgttgcggcgccatctgtcttcatctttcctccatctgatgaacaactcaagtctggaactgcttctgttgtgtgccttctgaacaacttctatcctagagaagccaaagtacagtggaaggttgacaatgctcttcaatcaggtaactcccaggagagtgtcacagagcaagattccaaggattccacctacagcctctcaagtaccttgacgttgagcaaggcagactatgagaaacacaaagtgtacgcatgcgaagtcactcatcagggcctgtcatcacccgtgacaaagagcttcaacaggggagagtgttaggtaccgagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 38 - DNA, 17686 bp, synthetic construct
  misc_feature 1..17686: note = pBYR11eMa-BAZE3-Hgp371
  source 1..17686: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtaagggcgtgtcatactccttgtgtaccgctgccttcacattcaccaagatcccggctgaaacactccacggaaccgttaccgtggaggtccaatacgccggtacagatggaccttgcaaggttccagctcagatggcggtggacatgcaaactcttaccccagttggaaggttgattaccgctaaccccgttatcactgaaagcactgagaactctaagatgatgttggaacttgatccaccattcggtgactcttacattgtcattggtgtgggagagaagaagatcacccaccactggcacaggagtggtagcactagtggtggatcaggaggttctggtggttctggaggttcaggatccgatgttcagcttcttgagtctggaggtggtcttgtgcaacctggaggttccttgagactctcctgtgcagcttcagggtttgacttcagtaggtactggatgagttgggttcgtcaagctcctgggaaaggactagaatggattggagagatcaatccagattcaagtaccatcaactatactccatctctgaaggatcgcttcaccatttccagagacaatgccaagaacacgttgtatcttcagatgaacagcttgaggactgaagacacagccttgtactactgcacaagacagggctatggctacaactactggggtcaaggcaccactgtcacagtgtcttcagctagcaccaaaggtccatcggtctttccactggcaccttcttccaagagtacttctggaggcacagctgcactgggttgtcttgtcaaggactactttccagaacctgttacggtttcgtggaactcaggtgctctgaccagtggagtgcacacctttccagctgttcttcagtcctcaggattgtattctcttagcagtgttgtgactgttccatcctcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtctccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaaggaggtggcggatcaggtggaggcggttcaggcggaggtggatcccataacactcctgtttacaagctggacatatctgaggcaactcaataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatcggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggatggtcttgcatcattctcttcttggtagccacagctacaggtgtccactccgatgttttgatgactcaaagccctctctcacttcctgtgactcttggacagcccgcatccatatcttgcagatctagtcagagtattgttcatagtaacggcaacacctacttggaatggtatctgcagaaaccaggccagtctccaaagcttctgatctacaaggcttccaatcgtttctctggtgtcccagacaggtttagtggcagtggatcagggactgacttcacattgaagatcagcagagttgaggctgaagatgcgggagtgtactattgtcttcaaggttcacatgttccgtcaacgtttggaggtgggaccaaagtggagatcaagactgttgcggcgccatctgtcttcatctttcctccatctgatgaacaactcaagtctggaactgcttctgttgtgtgccttctgaacaacttctatcctagagaagccaaagtacagtggaaggttgacaatgctcttcaatcaggtaactcccaggagagtgtcacagagcaagattccaaggattccacctacagcctctcaagtaccttgacgttgagcaaggcagactatgagaaacacaaagtgtacgcatgcgaagtcactcatcagggcctgtcatcacccgtgacaaagagcttcaacaggggagagtgttaggtaccgagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 39 - protein, 16 aa, synthetic construct
  REGION 1..16: note = linker sequence
GGSGGSGGSGGSGGSG

SEQ ID NO: 40 - protein, 12 aa, synthetic construct
  REGION 1..12: note = linker sequence
GTGGGGSGGGGS

SEQ ID NO: 41 - DNA, 14 bp, synthetic construct
  misc_feature 1..14: note = linker sequence
  source 1..14: mol_type = other DNA
ggsggsgssggsgg

SEQ ID NO: 42 - protein, 15 aa, synthetic construct
  REGION 1..15: note = linker sequence
SGGSGGSGGSGGSGS

SEQ ID NO: 43 - DNA, 14314 bp, synthetic construct
  misc_feature 1..14314: note = pBYR2eK2M-HBcheL2ic
  source 1..14314: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacaccatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagaggatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttggaggttctggtggatcaggaggttccggtggttctggaggttccggaatggacattgacccttacaaagaatttggagctactgtggagcttctcagctttttgccttctgacttctttccttctgtcagggatctccttgacactgcctcagctctttatagggaagccttggagtctcctgagcattgctcacctcaccatactgcactcaggcaagccattctctgctggggagaattgatgactcttgctacctgggtgggtaacaatctagagggtaccggtggaggcggttcaggcggaggtggatccgcaacccaactttacaagacttgcaaacaggctggaacatgtccacctgacattatcccaaaggtggaaggaaagaccattgctgatcagatcctccagtatggatcaatgggtgtgttctttggtggacttggaattggaacaggaagtggtacaggaggaaggactggttacatcccattgggaacaagacctccaacagctacagatacactggcaccagttagacctcctctaacagtagatccagttggaccatctgatccatctatcgtgtcccttgtagaggagacctctttcattgatgctggtgccccaactagtggaggttctggaggatctggttctagtggaggttctggtggagatccagcatccagagatcttgttgttaactatgttaatactaatgtgggtttgaagatcaggcaactcttgtggtttcatatatcttgccttacttttggaagagagactgtacttgaatatttggtctcttttggagtgtggattagaactcctccagcctatagaccaccaaatgcccctatcttgtcgactcttccagaaactactgttgttcgaagaagggacaggggcagatcccctagacgtagaactcccagccctagaagaaggagatccccatctcctaggcgtagataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 44 - DNA, 18066 bp, synthetic construct
  misc_feature 1..18066: note = pBYR11eMa-h6D8-L2
  source 1..18066: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggatggtcttgcatcatactctttcttgttgcaactgctacaggtgtccactctgatgttcagcttctcgagtctggaggtggtcttgtgcaacctggaggttccttgagactctcctgtgcagcttcagggtttgacttcagtaggtactggatgagttgggttcgtcaagctcctgggaaaggactagaatggattggagagatcaatccagattcaagtaccatcaactatactccatctctgaaggatcgcttcaccatttccagagacaatgccaagaacacgttgtatcttcagatgaacagcttgaggactgaagacacagccttgtactactgcacaagacagggctatggctacaactactggggtcaaggcaccactgtcacagtgtcttcagctagcaccaaaggtccatcggtctttccactggcaccttcttccaagagtacttctggaggcacagctgcactgggttgtcttgtcaaggactactttccagaacctgttacggtttcgtggaactcaggtgctctgaccagtggagtgcacacctttccagctgttcttcagtcctcaggattgtattctcttagcagtgttgtgactgttccatcctcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtctccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaaggaggtggcggatcaggtggaggcggttcaggcggaggtggatccgcaacccaactttacaagacttgcaaacaggctggaacatgtccacctgacattatcccaaaggtggaaggaaagaccattgctgatcagatcctccagtatggatcaatgggtgtgttctttggtggacttggaattggaacaggaagtggtacaggaggaaggactggttacatcccattgggaacaagacctccaacagctacagatacactggcaccagttagacctcctctaacagtagatccagttggaccatctgatccatctatcgtgtcccttgtagaggagacctctttcattgatgctggtgccccaactagtcataacactcctgtttacaagctggacatatctgaggcaactcaataagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattccaaagaattatagaagggatcggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatgggatggtcttgcatcattctcttcttggtagccacagctacaggtgtccactccgatgttttgatgactcaaagccctctctcacttcctgtgactcttggacagcccgcatccatatcttgcagatctagtcagagtattgttcatagtaacggcaacacctacttggaatggtatctgcagaaaccaggccagtctccaaagcttctgatctacaaggcttccaatcgtttctctggtgtcccagacaggtttagtggcagtggatcagggactgacttcacattgaagatcagcagagttgaggctgaagatgcgggagtgtactattgtcttcaaggttcacatgttccgtcaacgtttggaggtgggaccaaagtggagatcaagactgttgcggcgccatctgtcttcatctttcctccatctgatgaacaactcaagtctggaactgcttctgttgtgtgccttctgaacaacttctatcctagagaagccaaagtacagtggaaggttgacaatgctcttcaatcaggtaactcccaggagagtgtcacagagcaagattccaaggattccacctacagcctctcaagtaccttgacgttgagcaaggcagactatgagaaacacaaagtgtacgcatgcgaagtcactcatcagggcctgtcatcacccgtgacaaagagcttcaacaggggagagtgttaggtaccgagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg

SEQ ID NO: 45 - DNA, 14156 bp, synthetic construct
  misc_feature 1..14156: note = pBYe3R2K2Mc-BAZsE6H
  source 1..14156: mol_type = other DNA
cgatcggtcgattcatagaagattagatttttcatagtatttttttaaagtaaacctttaactacggttaggacacttttaagttaaatttaatttgaacccttaaattaatttttaaaatagataaatatcaatcatcctgatatgcttttgaaaaaatgaatgagaaagatgattcaattaaggccacattttaatcatgactaaaataatatacagtataatttcatatatatttgctttaaaaaaaaattgacaatccattcgtttctagcaataaatttcttcaaccacaaatatattaaagataactacggcatagaaacaaaaatctatgaagaatttttgtatacttcatatgaaattaaaaaaaacttcattgaacatcaaaataataataataatcataaactcctcaatatttatattcctagcttcttgaattaaattgtttacatattcaacgatgtaaaaaattatttctctatctattttccttatatcatgcatggtttcacatatatcaaaggataaaagcaatctatgtaaattatctcactttattaagttttctatctgaattattgagaacgtagatttctttttgcactatcccccaataattagcaaaacacacctagactagatttgttttgctaacccaattgatattaattatatatgattaatatttatatgtatatggaattggttaataaaatgcatctggttcatcaaagaattataaagacacgtgacattcatttaggataagaaatatggatgatctctttctcttattcagataattagtaattacacataacacacaactttgatgcccacattatagtgattagcatgtcactatgtgtgcatccttttatttcatacattaattaacttggccaatccagaagatggacaagtctagggtcacattgcagggtactctagcttactcgccttctttttcgaaggtttgagtaccttcagggcatcctcttgatacattactttccacttcgattggggcaagctgtagcagttcttgcttagaccgaattgccatctcacagagatgctgaagagttcgcgaccctccagaaacggtgatactaactcctcgaaaccgaatactataggtacatccgatctggtcgaaaccgaaaaatcgagatgctgcatagttaaccgaatctcccgtccaagatccaaggactctgtgcagtgaagcttccgtcctgtcgtatctgagatatctcttaaatacaactttcccgaaaccccagctttccttgaaaccaaggggattatcttgattcgaattcgtctcatcgttatgtagccgccactcagtccaactcggactttcgtcaggaagtttgaagggagaagttgtacctcctgatcctccatcccaacgttcactgttagcttgttccctagcgtcgtttccttgtatagctcgttccatggattgtaaatagtaattgtaatgttgtttgttgtttgttgttgttggtaattgttgtaaaaatacgctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctggcgcgccgctctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacgaccctcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagaaacaaacaaaatcaacaaatatagaaaataacgcatttccaattctttgaaatttctgcaacatctagaacaatggctaacaagcacctctcattgtctctcttccttgtgctccttggtctttctgcttctcttgcttctggtatcaggtgcattggagtgagcaacagggactttgtggaaggtatgtcaggtggaacttgggttgatgttgtgttggaacatgggggttgtgtcaccgtgatggcccaggacaaaccgactgtcgacattgagttggttacaacaacggtcagcaacatggccgaggttagatcctactgctatgaggcttcaatttcagacatggctagtgacagccgttgcccaacacaaggtgaagcctaccttgacaagcaatcagacactcaatatgtgtgcaagagaacattggtggacagaggttggggaaacggatgtggacttttcggtaagggaagcctcgtgacatgcgctaaattcgcttgctccaagaagatgaccggaaagagcatccagccagagaacctcgagtaccggattatgttgtcagttcatggttcccagcacagcggaatgatcgttaatgacacaggacatgaaactgatgagaatagagccaaggttgagattacacctaactcaccaagagccgaagccaccctcggaggtttcggaagcttgggacttgattgtgaaccgaggacaggccttgacttttcagatttgtactacttgactatgaataacaagcactggttggttcacaaggaatggttccacgacattccattgccttggcacgctggtgctgacaccggaactccacactggaacaacaaagaggcactcgtggaattcaaggacgcccatgccaagaggcaaactgtcgtggttcttggtactcaagaaggagccgttcacacagcccttgctggtgctctcgaggctgagatggatggtgctaagggaaggctttcctctggccacttgaaatgtcgtttgaagatggataagcttagattgaagggcgtgtcatactccttgtgtaccgctgccttcacattcaccaagatcccggctgaaacactccacggaaccgttaccgtggaggtccaatacgccggtacagatggaccttgcaaggttccagctcagatggcggtggacatgcaaactcttaccccagttggaaggttgattaccgctaaccccgttatcactgaaagcactgagaactctaagatgatgttggaacttgatccaccattcggtgactcttacattgtcattggtgtgggagagaagaagatcacccaccactggcacaggagtggtagcactagtcaccatcaccatcaccattaagagctcgaagtgacatcacaaagttgaaggtaataaagccaaattaattaagacattttcataatgatgtcaagaatgcaaagcaaattgcataactgcctttatgcaaaacattaatataatataaattataaagaactgcgctctctgcttcttattttcttagcttcatttattagtcactagctgttcagaattttcagtatcttttgatattactaagaacctaatcacacaatgtatattcttatgcaggaaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccctaaaaccaaaatccagtgacgaattctcgattaaaaatcccaattatatttggtctaatttagtttggtattgagtaaaacaaattcgaaccaaaccaaaatataaatatatagtttttatatatatgcctttaagactttttatagaattttctttaaaaaatatctaggtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggtaccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaactcttctttgatcatgtcaagatactcctccttagacgttgcagtctggataatagttcgccatcgtgcgtcagatttgcgaggagacaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgtggttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggaccggtcccctaggccggccaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggagactgtcatacgcgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcgagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaaggg